SEQUENCE LISTING (71 sequences)

SEQ ID NO: 1 - DNA, 1248 bp, Chondromyces crocatus
ctggcgctggcgctgcgcctgaccggtgccctcgaccgggtggcgctgcaggcggccctcggcgatgtcgtcgcgcgccacgaaagcttgcggacggtgttcccgcacgccgacgggaccccctcccaggtggtgctcgatgccgacgcggcgcgccccgcgctcaccgtcacccggaccgacgcggagagcgtacgtgacgcgctgaacacggcggtgcgtcatggcttcgatctgtccgtcgagccaccgctgcgggccacgctgttcgaggtggcgcccgaggtccacgtgctgctgctgacgatgcaccacatcgtcggtgacggcggctcgatggaacccctttcgcaggacctggccaccgcgtatgccgcgcgctgccagggggaagcgccggcctggtcgccgcttccggtgcagtacgccgactacacgctctggcagcgggagctgctcggcgaccaggccgacgccgagagccggttcgcgcagcagctcgcctactggaccagggaactggcgggcctccccgagcagctcacgctacccaccgaccgcccgcgcccgcgggtggcctcctaccggggaggggtggtccagatggcgtgggacgcctccttgcaccagggcctgatcgccctcgcgcgcaagaacggcgccagcttgttcatggtgctccaggctggcctcgccgccttgttcatgcggctgggagcgggtcacgacatcgcgctgggcagcccgatcgcgggtcgcaccgaccatgcgctcgacgacctggtcgggttcttcgtcaacacgctggtgctgcgcgcggacacgtcggggaacccgagcttccggcagctgctgtgccgcgctcgtggagtggccctggccgcctacgcccatcaggacgtgccgttcgagtgcctggtcgaggcgttgaacccgacgcgatcgctggcacaccacccgctgttccaggtcatgctcggcgtgcagcgcgcccagccgaaggacatcgagctgtctggtctgcacgtcgagccggcagagaccggcaccacggccaccgcgcgcgtcgacctgacgttcagcgtcaccgagcgccgcagcgccgagggcgctgcggagggcatcgagggggtggtcgagtacagcagcgatctgttcgacgccgcctcggtcgagacgctggtggcgcggtgggcgcggctgctggaggccgccgtcgcggatccggagcagcccatcgggaacctg

SEQ ID NO: 2 - protein, 416 aa, Chondromyces crocatus
LALALRLTGALDRVALQAALGDVVARHESLRTVFPHADGTPSQVVLDADAARPALTVTRTDAESVRDALNTAVRHGFDLSVEPPLRATLFEVAPEVHVLLLTMHHIVGDGGSMEPLSQDLATAYAARCQGEAPAWSPLPVQYADYTLWQRELLGDQADAESRFAQQLAYWTRELAGLPEQLTLPTDRPRPRVASYRGGVVQMAWDASLHQGLIALARKNGASLFMVLQAGLAALFMRLGAGHDIALGSPIAGRTDHALDDLVGFFVNTLVLRADTSGNPSFRQLLCRARGVALAAYAHQDVPFECLVEALNPTRSLAHHPLFQVMLGVQRAQPKDIELSGLHVEPAETGTTATARVDLTFSVTERRSAEGAAEGIEGVVEYSSDLFDAASVETLVARWARLLEAAVADPEQPIGNL

SEQ ID NO: 3 - DNA, 1569 bp, Chondromyces crocatus
gtcctgacggctgacgagcgccggaggctgctggtcgaccacaacgcgaccgcccatccggtcgcggccatcagcctgagcgcagcgttccaggcgcaggtggaggcgacgccggacgcggtggcggtggtgtgcgacggcacggcgctgacgtacgccgagctgaacgcgcgggcgaaccggctggcgcaccagctgatcgcgcagggggtagcgctggagagccgtgtggcgctggcgctggagcggtcgctggagctggtgctggccctgctggccgtcatcaaggccgggggagcttacgtgcccctggatgcgcgctacccgcaggcgcggagagcgcacatcctgaaggaaacgggcgcagtggtgctgctggccagcggggaggggagcgacgacaccgcgtcgctgggcgtcccggtgctgctggtcgacgctggttccgtcgcgtccgatccgggcgcgccggttgtcgtctgcgatccggaccagctcgcgtacgtcatgtacacgtcagggtcgacggggcagccgaaggggatcggcgtcacgcaccggaacgtggtggagctggcctcggatccgtgctggcgctcggggcatcaacggcgggtgctgtggcactcaccgccggcgttcgacgcctcgacctacgagttctgggtgccgctcctgggtggcgggcagatcgtcgtttcacccgctggtgagcagaccgcccatgatctccggcgcgtgatctccgagcaccaggtcaccagcgtcttcctgacgacggcgctgttcaacctgatggtggaggaagacccgagcagcttccacacggtgggcgaagtgtggaccggcggcgaagcggtctcgccgcagtcgatgcaacgggtgctggacacctgcccggacacgatgatcgcccacgtctacggcccgacggagacgacgacgttcgccacgttcgaggccctgcgaccgccgcaccacatcgagggcacggtgccgatcggcaagccgatggcgaacatgcgggcttacgtgctcgatgaaggcttgcggcccgtgccagaaggcgtgcccggggagctgtacctcgcgggcgccgggctctcgcgcggatacgtcgcgcgccctggactgacggccgagcgcttcgtcgtcgacccgttcgccagcggcgagcgcatgtaccgcaccggcgatcgtgtccggtggaacgctggcgggagcctcgacttcctgggccgcaccgacaaccaggtgaagatccgaggcttccgcatcgagccggacgagatcggcgcggtgctgctggagcatcccgaggtcgcgcaggcggcggtcgtcgtccgcgaggaccggcctggcgagaagcggctgatcgcttacgccgtcgccaccgcggggacgaaccccgacccgcgggcgctgcgcgactggagcaagcagcggctgccggagttcatggtgcccgccgcgctcgtcctgctcgacgccttgccgctgaacgcgaacggcaagctcgaccgcaaggcgctgccggcccccgatctcggaccgtctcgcgctggcaga

SEQ ID NO: 4 - protein, 523 aa, Chondromyces crocatus
VLTADERRRLLVDHNATAHPVAAISLSAAFQAQVEATPDAVAVVCDGTALTYAELNARANRLAHQLIAQGVALESRVALALERSLELVLALLAVIKAGGAYVPLDARYPQARRAHILKETGAVVLLASGEGSDDTASLGVPVLLVDAGSVASDPGAPVVVCDPDQLAYVMYTSGSTGQPKGIGVTHRNVVELASDPCWRSGHQRRVLWHSPPAFDASTYEFWVPLLGGGQIVVSPAGEQTAHDLRRVISEHQVTSVFLTTALFNLMVEEDPSSFHTVGEVWTGGEAVSPQSMQRVLDTCPDTMIAHVYGPTETTTFATFEALRPPHHIEGTVPIGKPMANMRAYVLDEGLRPVPEGVPGELYLAGAGLSRGYVARPGLTAERFVVDPFASGERMYRTGDRVRWNAGGSLDFLGRTDNQVKIRGFRIEPDEIGAVLLEHPEVAQAAVVVREDRPGEKRLIAYAVATAGTNPDPRALRDWSKQRLPEFMVPAALVLLDALPLNANGKLDRKALPAPDLGPSRAGR

SEQ ID NO: 5 - DNA, 195 bp, Chondromyces crocatus
cacctgctctgcgatctcttcgccgaggtcctcggcctgccgcgcgtcagcatcgacgacgacttcttcgagctgggcggccactcgctgctcgccacccgcctcgtcagccgcgtgcgcaccaccctcggcgtcgagctgagcgtccgcagcctcttcgagagtcccaccgtggccgggctgtgcggccgtctg

SEQ ID NO: 6 - protein, 65 aa, Chondromyces crocatus
HLLCDLFAEVLGLPRVSIDDDFFELGGHSLLATRLVSRVRTTLGVELSVRSLFESPTVAGLCGRL

SEQ ID NO: 7 - DNA, 1326 bp, Chondromyces crocatus
ccggaccgccttcccctgtcgttcgcgcagcagcgcctgtggttcttgcaccagatggaaggccgctctgcgacctacaacatccccatggccctgcgtctgacggggacactcgaccgcgcggcgctggaggccgcactgggcgacgtggtcacccgtcacgagagcctccggacgaggttctctcagcacgacggcaccgcctaccaggccatcctggctcccaccgaggcgcgcccgtcgctgtccgtcaccgtgaccacggatgcggagctgccggaggccctggccgcggccgctcagtacggcttcgacctcgcgcacgagctgccgctgcgcgccgagctgttcgtgctgggccctggcgagcacctgctgctgctcctgctgcatcacatcgccggtgatggctggtccctcgcgcccttgtcgcgcgacctcgcgaccgcgtacacggcccggtgcggaggcgaagcgccggcgtggacgccgttgccggtccagtacggcgactacaccctctggcagcacgccttgctgggaggcgtcgccgatcccgacagcctgttcagccgccagctcgcgtactggacccggaccctcgctgatctccccgagcgcatcgagctgcccgccgatcgcccgggcccggcggtcgcctcgtaccggggcgactacctccccgtgcagatcgacgccgccctgcaccgcggcctgcacggcctcgcccgacagagcggcgccagcctgttcatggtgctccaggccggactcgcggcgctcctgtctcgcctcggcgcgggcgacgacatccccctgggcagccccatcgccgggcgcacggatcgcgcgctggaggacctggtcggcttcttcgtcaacaccctggtgctgcgcacggacacctcggggaatcccagcttccgacagctcctcggccgcgtgcgggagacggcgctcagcgcctacgcccaccaggacatgccgttcgagcacctcgtcgagatcctcaaccctgccaggtcgctctcgcaccaccccctgttccaggtgctgctcgcggtccagaacgcgcctgaaggcgccttcacgctgcctggcctggacgtctccttcgtctccacccgcaccggcacctccaagttcgacctcggcttcagcctgtccgaacagcgcggcgcggacggttccccgcaagggctggccggctacgtcgagtacagcaccgaccgcttcgacctcggcaccgtcgagaccctgttctcgcgctggatccgcttgctggaggctgcggtggagcacccggatcgcccgatcggggccacc

SEQ ID NO: 8 - protein, 442 aa, Chondromyces crocatus
PDRLPLSFAQQRLWFLHQMEGRSATYNIPMALRLTGTLDRAALEAALGDVVTRHESLRTRFSQHDGTAYQAILAPTEARPSLSVTVTTDAELPEALAAAAQYGFDLAHELPLRAELFVLGPGEHLLLLLLHHIAGDGWSLAPLSRDLATAYTARCGGEAPAWTPLPVQYGDYTLWQHALLGGVADPDSLFSRQLAYWTRTLADLPERIELPADRPGPAVASYRGDYLPVQIDAALHRGLHGLARQSGASLFMVLQAGLAALLSRLGAGDDIPLGSPIAGRTDRALEDLVGFFVNTLVLRTDTSGNPSFRQLLGRVRETALSAYAHQDMPFEHLVEILNPARSLSHHPLFQVLLAVQNAPEGAFTLPGLDVSFVSTRTGTSKFDLGFSLSEQRGADGSPQGLAGYVEYSTDRFDLGTVETLFSRWIRLLEAAVEHPDRPIGAT

SEQ ID NO: 9 - DNA, 1557 bp, Chondromyces crocatus
cgccacaccctcctcgtcgagcgcaacgacaccgcccagcccctccccgaggccacgttcccgaccctcttccaggcacaggtcgaggcgacgcccggggcagtggcgctggcatgggacgaggcccagctcacctacggcgagctgaacgcccgggccaaccagcttgcgcacaggctgcgcgcggaaggcgtgggacccgagcacctcgtggccctggccatgccccgctcacccgacctggtgatcgcccttctggccgtgctgaaggccggcgcggcctacctcccggtggacccggactaccccgccgcgcggatcgccttcatgctcaccgacgcccggcccatcctgctgctgacccgcctcgacacgcccgcggccgcgttcgagagcatccccacgcccaggctggtggtcgacgaccccgccacgatccgcgcgctcgccgatctccccgccagcaacccggtggtggccgtgctgccgcagcaccccgcgtacgtcatctacacctcgggctcgaccggagttcccaagggcgtggtcgtgagccaccagggcatcgccagcctggcgaaggcccacatcgagcggttcggtgtgaccgcgcagagccgcgtgctccagttcgcctcgcccagcttcgatgcctcgttcgcggacctggccatgaccttcctttcgggcgcggcgctggtgctggcaccgaaggaacagctgcagccgggcgctccgctggccgcgctgacgagccgacagcgggtgacgcacgcgacgctccccccggccgccctctcgatcatgtcaccgcagggcggcctccccgctgacatgaccctggtcgtggccggcgaggcctgcccgcccgagctggtcgcagcctgggcacccgggcgacggatgatcaacgcctacggccccaccgagaccacggtctgcgccacactgagcgagctgttgccgcccgccgcagccatcccacccatcgggagacccatcgtgaacaccagggtctacgtgctcgatgcgggcctccagcccgtgcctcccggcgtggccggggagctctacgtcgccggcgcgggtctggcacggggctacctgggcaggccaggcttgacggcggcgcgcttcgtcgcgagccccttcggcgacggcgcgcgcatgtaccgcaccggcgaccgggcgcgctggaacgcggacgggagcctcgagttttgcggacgagccgacgatcaggtcaagcttcgcggcttccggatcgagctcggcgagatcgaagcccagctctccgcgcaccccgaggtcgcgcaggccgccgtggtggtccgccaggatggccaggctgccgacaggcgcctggtcgcctacgtcgtcgccgcagagcgggacggcaaggaccgcaacgagcagatcgagcacgaccaggtgcgcgcgtggcagcagatctacgagacccactacgcgaccgtggacgcgacccggttcgggcaggacttcagcggctggaacagcagctacgacggagagcccatcccggtcgagcagatgcgc

SEQ ID NO: 10 - protein, 519 aa, Chondromyces crocatus
RHTLLVERNDTAQPLPEATFPTLFQAQVEATPGAVALAWDEAQLTYGELNARANQLAHRLRAEGVGPEHLVALAMPRSPDLVIALLAVLKAGAAYLPVDPDYPAARIAFMLTDARPILLLTRLDTPAAAFESIPTPRLVVDDPATIRALADLPASNPVVAVLPQHPAYVIYTSGSTGVPKGVVVSHQGIASLAKAHIERFGVTAQSRVLQFASPSFDASFADLAMTFLSGAALVLAPKEQLQPGAPLAALTSRQRVTHATLPPAALSIMSPQGGLPADMTLVVAGEACPPELVAAWAPGRRMINAYGPTETTVCATLSELLPPAAAIPPIGRPIVNTRVYVLDAGLQPVPPGVAGELYVAGAGLARGYLGRPGLTAARFVASPFGDGARMYRTGDRARWNADGSLEFCGRADDQVKLRGFRIELGEIEAQLSAHPEVAQAAVVVRQDGQAADRRLVAYVVAAERDGKDRNEQIEHDQVRAWQQIYETHYATVDATRFGQDFSGWNSSYDGEPIPVEQMR

SEQ ID NO: 11 - DNA, 1230 bp, Chondromyces crocatus
cgggacggcaaggaccgcaacgagcagatcgagcacgaccaggtgcgcgcgtggcagcagatctacgagacccactacgcgaccgtggacgcgacccggttcgggcaggacttcagcggctggaacagcagctacgacggagagcccatcccggtcgagcagatgcgcgagtggcgcgacgccaccgtcacccgcatcctctcgctgcgcccgaggcgcgtcctggagatcggggtcggcaacgcgctgctcctctcgcagatcgcgccccactgcgagagctactggggcaccgacctctcggccacggtcatcgcctcgctggcgacgcagctcgagcacctgcccgagctgtcggagaaggtcgtgctgcgcgcccagcccgcccacgacctcggcgggctgcccgcgggaacgttcgacacgatcgtcatcaactcggtcgtgcagtacttccccaacaccgactacctcgtcgacgtgctgaaccaggcgctccagctcctcgtccctggtggggcgctgttcgtcggcgatgtgcgcaacgtgcagctcctgcgctgcttcgccaccgccgtccagcttcgccgcgccgaggacggcgcggaggaggccgcgctgcgccacgcgatcgagcacgccctgcgggtggagaaggagctgctcgtcgcgcccgagttcttcgcggccctcgcggcgtcgcatccggacatcggtggcgtggacgtccgcctcaagcgcggccagcaccacaacgagctgacccgctaccgctacgacgccatcctgcgaaaatcacccatcccagcgctctcgctggccgaggcccccacgctgcgatgggaagcgtgcggcggcatcccagccctcgaagcgctgctcgcgggcgagcgccccgaccggctacgcctgagtggcgtcccgaaccgccgcatccaccaggaagccgccgccctgcgcgtcttcgaggaaggccatcccgtgagcgcatcgcggaagctcctggaggacagcctcccggaggcgctcgatccagagtccctcgtcgcgctgggagaacgtcacggctactgggtggccgtcacctggtcgccgacctcggtcgacgccgtcgacgtcctgttcgtgcaggccgagacggtagcctcggctgcacccgtcgacgtccacacgccctccggcatcgcgggcatgccgctgtccgcgttcacgaacaacccctcgaccgcg

SEQ ID NO: 12 - protein, 410 aa, Chondromyces crocatus
RDGKDRNEQIEHDQVRAWQQIYETHYATVDATRFGQDFSGWNSSYDGEPIPVEQMREWRDATVTRILSLRPRRVLEIGVGNALLLSQIAPHCESYWGTDLSATVIASLATQLEHLPELSEKVVLRAQPAHDLGGLPAGTFDTIVINSVVQYFPNTDYLVDVLNQALQLLVPGGALFVGDVRNVQLLRCFATAVQLRRAEDGAEEAALRHAIEHALRVEKELLVAPEFFAALAASHPDIGGVDVRLKRGQHHNELTRYRYDAILRKSPIPALSLAEAPTLRWEACGGIPALEALLAGERPDRLRLSGVPNRRIHQEAAALRVFEEGHPVSASRKLLEDSLPEALDPESLVALGERHGYWVAVTWSPTSVDAVDVLFVQAETVASAAPVDVHTPSGIAGMPLSAFTNNPSTA

SEQ ID NO: 13 - DNA, 195 bp, Chondromyces crocatus
cagatgctgtgcgacctgttcgccgaggtgctggggctgggggaggtgggcatcgacgaggacttcttcgcgctgggcggtcactcgctgctggcgacgcgattgatcggccggatccgcgccaccctgggtgtggaggtgccgctccgagcgctgttcgaagcgccgacggtggcccgtctggccacccagctc

SEQ ID NO: 14 - protein, 65 aa, Chondromyces crocatus
QMLCDLFAEVLGLGEVGIDEDFFALGGHSLLATRLIGRIRATLGVEVPLRALFEAPTVARLATQL

SEQ ID NO: 15 - DNA, 7467 bp, Chondromyces crocatus
ctggcgctggcgctgcgcctgaccggtgccctcgaccgggtggcgctgcaggcggccctcggcgatgtcgtcgcgcgccacgaaagcttgcggacggtgttcccgcacgccgacgggaccccctcccaggtggtgctcgatgccgacgcggcgcgccccgcgctcaccgtcacccggaccgacgcggagagcgtacgtgacgcgctgaacacggcggtgcgtcatggcttcgatctgtccgtcgagccaccgctgcgggccacgctgttcgaggtggcgcccgaggtccacgtgctgctgctgacgatgcaccacatcgtcggtgacggcggctcgatggaacccctttcgcaggacctggccaccgcgtatgccgcgcgctgccagggggaagcgccggcctggtcgccgcttccggtgcagtacgccgactacacgctctggcagcgggagctgctcggcgaccaggccgacgccgagagccggttcgcgcagcagctcgcctactggaccagggaactggcgggcctccccgagcagctcacgctacccaccgaccgcccgcgcccgcgggtggcctcctaccggggaggggtggtccagatggcgtgggacgcctccttgcaccagggcctgatcgccctcgcgcgcaagaacggcgccagcttgttcatggtgctccaggctggcctcgccgccttgttcatgcggctgggagcgggtcacgacatcgcgctgggcagcccgatcgcgggtcgcaccgaccatgcgctcgacgacctggtcgggttcttcgtcaacacgctggtgctgcgcgcggacacgtcggggaacccgagcttccggcagctgctgtgccgcgctcgtggagtggccctggccgcctacgcccatcaggacgtgccgttcgagtgcctggtcgaggcgttgaacccgacgcgatcgctggcacaccacccgctgttccaggtcatgctcggcgtgcagcgcgcccagccgaaggacatcgagctgtctggtctgcacgtcgagccggcagagaccggcaccacggccaccgcgcgcgtcgacctgacgttcagcgtcaccgagcgccgcagcgccgagggcgctgcggagggcatcgagggggtggtcgagtacagcagcgatctgttcgacgccgcctcggtcgagacgctggtggcgcggtgggcgcggctgctggaggccgccgtcgcggatccggagcagcccatcgggaacctggaggtcctgacggctgacgagcgccggaggctgctggtcgaccacaacgcgaccgcccatccggtcgcggccatcagcctgagcgcagcgttccaggcgcaggtggaggcgacgccggacgcggtggcggtggtgtgcgacggcacggcgctgacgtacgccgagctgaacgcgcgggcgaaccggctggcgcaccagctgatcgcgcagggggtagcgctggagagccgtgtggcgctggcgctggagcggtcgctggagctggtgctggccctgctggccgtcatcaaggccgggggagcttacgtgcccctggatgcgcgctacccgcaggcgcggagagcgcacatcctgaaggaaacgggcgcagtggtgctgctggccagcggggaggggagcgacgacaccgcgtcgctgggcgtcccggtgctgctggtcgacgctggttccgtcgcgtccgatccgggcgcgccggttgtcgtctgcgatccggaccagctcgcgtacgtcatgtacacgtcagggtcgacggggcagccgaaggggatcggcgtcacgcaccggaacgtggtggagctggcctcggatccgtgctggcgctcggggcatcaacggcgggtgctgtggcactcaccgccggcgttcgacgcctcgacctacgagttctgggtgccgctcctgggtggcgggcagatcgtcgtttcacccgctggtgagcagaccgcccatgatctccggcgcgtgatctccgagcaccaggtcaccagcgtcttcctgacgacggcgctgttcaacctgatggtggaggaagacccgagcagcttccacacggtgggcgaagtgtggaccggcggcgaagcggtctcgccgcagtcgatgcaacgggtgctggacacctgcccggacacgatgatcgcccacgtctacggcccgacggagacgacgacgttcgccacgttcgaggccctgcgaccgccgcaccacatcgagggcacggtgccgatcggcaagccgatggcgaacatgcgggcttacgtgctcgatgaaggcttgcggcccgtgccagaaggcgtgcccggggagctgtacctcgcgggcgccgggctctcgcgcggatacgtcgcgcgccctggactgacggccgagcgcttcgtcgtcgacccgttcgccagcggcgagcgcatgtaccgcaccggcgatcgtgtccggtggaacgctggcgggagcctcgacttcctgggccgcaccgacaaccaggtgaagatccgaggcttccgcatcgagccggacgagatcggcgcggtgctgctggagcatcccgaggtcgcgcaggcggcggtcgtcgtccgcgaggaccggcctggcgagaagcggctgatcgcttacgccgtcgccaccgcggggacgaaccccgacccgcgggcgctgcgcgactggagcaagcagcggctgccggagttcatggtgcccgccgcgctcgtcctgctcgacgccttgccgctgaacgcgaacggcaagctcgaccgcaaggcgctgccggcccccgatctcggaccgtctcgcgctggcagagcgccacgaacccagcgcgagcacctgctctgcgatctcttcgccgaggtcctcggcctgccgcgcgtcagcatcgacgacgacttcttcgagctgggcggccactcgctgctcgccacccgcctcgtcagccgcgtgcgcaccaccctcggcgtcgagctgagcgtccgcagcctcttcgagagtcccaccgtggccgggctgtgcggccgtctggagagggacgacgccagcaccgtgcgcctggccttgcgcgcccaggcccgtccggaccgccttcccctgtcgttcgcgcagcagcgcctgtggttcttgcaccagatggaaggccgctctgcgacctacaacatccccatggccctgcgtctgacggggacactcgaccgcgcggcgctggaggccgcactgggcgacgtggtcacccgtcacgagagcctccggacgaggttctctcagcacgacggcaccgcctaccaggccatcctggctcccaccgaggcgcgcccgtcgctgtccgtcaccgtgaccacggatgcggagctgccggaggccctggccgcggccgctcagtacggcttcgacctcgcgcacgagctgccgctgcgcgccgagctgttcgtgctgggccctggcgagcacctgctgctgctcctgctgcatcacatcgccggtgatggctggtccctcgcgcccttgtcgcgcgacctcgcgaccgcgtacacggcccggtgcggaggcgaagcgccggcgtggacgccgttgccggtccagtacggcgactacaccctctggcagcacgccttgctgggaggcgtcgccgatcccgacagcctgttcagccgccagctcgcgtactggacccggaccctcgctgatctccccgagcgcatcgagctgcccgccgatcgcccgggcccggcggtcgcctcgtaccggggcgactacctccccgtgcagatcgacgccgccctgcaccgcggcctgcacggcctcgcccgacagagcggcgccagcctgttcatggtgctccaggccggactcgcggcgctcctgtctcgcctcggcgcgggcgacgacatccccctgggcagccccatcgccgggcgcacggatcgcgcgctggaggacctggtcggcttcttcgtcaacaccctggtgctgcgcacggacacctcggggaatcccagcttccgacagctcctcggccgcgtgcgggagacggcgctcagcgcctacgcccaccaggacatgccgttcgagcacctcgtcgagatcctcaaccctgccaggtcgctctcgcaccaccccctgttccaggtgctgctcgcggtccagaacgcgcctgaaggcgccttcacgctgcctggcctggacgtctccttcgtctccacccgcaccggcacctccaagttcgacctcggcttcagcctgtccgaacagcgcggcgcggacggttccccgcaagggctggccggctacgtcgagtacagcaccgaccgcttcgacctcggcaccgtcgagaccctgttctcgcgctggatccgcttgctggaggctgcggtggagcacccggatcgcccgatcggggccaccgagctgctctccgcgcgcgagcgccacaccctcctcgtcgagcgcaacgacaccgcccagcccctccccgaggccacgttcccgaccctcttccaggcacaggtcgaggcgacgcccggggcagtggcgctggcatgggacgaggcccagctcacctacggcgagctgaacgcccgggccaaccagcttgcgcacaggctgcgcgcggaaggcgtgggacccgagcacctcgtggccctggccatgccccgctcacccgacctggtgatcgcccttctggccgtgctgaaggccggcgcggcctacctcccggtggacccggactaccccgccgcgcggatcgccttcatgctcaccgacgcccggcccatcctgctgctgacccgcctcgacacgcccgcggccgcgttcgagagcatccccacgcccaggctggtggtcgacgaccccgccacgatccgcgcgctcgccgatctccccgccagcaacccggtggtggccgtgctgccgcagcaccccgcgtacgtcatctacacctcgggctcgaccggagttcccaagggcgtggtcgtgagccaccagggcatcgccagcctggcgaaggcccacatcgagcggttcggtgtgaccgcgcagagccgcgtgctccagttcgcctcgcccagcttcgatgcctcgttcgcggacctggccatgaccttcctttcgggcgcggcgctggtgctggcaccgaaggaacagctgcagccgggcgctccgctggccgcgctgacgagccgacagcgggtgacgcacgcgacgctccccccggccgccctctcgatcatgtcaccgcagggcggcctccccgctgacatgaccctggtcgtggccggcgaggcctgcccgcccgagctggtcgcagcctgggcacccgggcgacggatgatcaacgcctacggccccaccgagaccacggtctgcgccacactgagcgagctgttgccgcccgccgcagccatcccacccatcgggagacccatcgtgaacaccagggtctacgtgctcgatgcgggcctccagcccgtgcctcccggcgtggccggggagctctacgtcgccggcgcgggtctggcacggggctacctgggcaggccaggcttgacggcggcgcgcttcgtcgcgagccccttcggcgacggcgcgcgcatgtaccgcaccggcgaccgggcgcgctggaacgcggacgggagcctcgagttttgcggacgagccgacgatcaggtcaagcttcgcggcttccggatcgagctcggcgagatcgaagcccagctctccgcgcaccccgaggtcgcgcaggccgccgtggtggtccgccaggatggccaggctgccgacaggcgcctggtcgcctacgtcgtcgccgcagagcgggacggcaaggaccgcaacgagcagatcgagcacgaccaggtgcgcgcgtggcagcagatctacgagacccactacgcgaccgtggacgcgacccggttcgggcaggacttcagcggctggaacagcagctacgacggagagcccatcccggtcgagcagatgcgcgagtggcgcgacgccaccgtcacccgcatcctctcgctgcgcccgaggcgcgtcctggagatcggggtcggcaacgcgctgctcctctcgcagatcgcgccccactgcgagagctactggggcaccgacctctcggccacggtcatcgcctcgctggcgacgcagctcgagcacctgcccgagctgtcggagaaggtcgtgctgcgcgcccagcccgcccacgacctcggcgggctgcccgcgggaacgttcgacacgatcgtcatcaactcggtcgtgcagtacttccccaacaccgactacctcgtcgacgtgctgaaccaggcgctccagctcctcgtccctggtggggcgctgttcgtcggcgatgtgcgcaacgtgcagctcctgcgctgcttcgccaccgccgtccagcttcgccgcgccgaggacggcgcggaggaggccgcgctgcgccacgcgatcgagcacgccctgcgggtggagaaggagctgctcgtcgcgcccgagttcttcgcggccctcgcggcgtcgcatccggacatcggtggcgtggacgtccgcctcaagcgcggccagcaccacaacgagctgacccgctaccgctacgacgccatcctgcgaaaatcacccatcccagcgctctcgctggccgaggcccccacgctgcgatgggaagcgtgcggcggcatcccagccctcgaagcgctgctcgcgggcgagcgccccgaccggctacgcctgagtggcgtcccgaaccgccgcatccaccaggaagccgccgccctgcgcgtcttcgaggaaggccatcccgtgagcgcatcgcggaagctcctggaggacagcctcccggaggcgctcgatccagagtccctcgtcgcgctgggagaacgtcacggctactgggtggccgtcacctggtcgccgacctcggtcgacgccgtcgacgtcctgttcgtgcaggccgagacggtagcctcggctgcacccgtcgacgtccacacgccctccggcatcgcgggcatgccgctgtccgcgttcacgaacaacccctcgaccgcgcgagggaccggggcactgatcgccaccctccgggagcacctccgcgagcggctccccgactacatggtgcccgcagccgtggtcgtcctggagcgctttccgctctcccccagcggcaagctcgaccgccaggcgctgcctgcgccggagctgggtcaggaccgcgcgggacgagcggcgcgcacgccccaggaacagatgctgtgcgacctgttcgccgaggtgctggggctgggggaggtgggcatcgacgaggacttcttcgcgctgggcggtcactcgctgctggcgacgcgattgatcggccggatccgcgccaccctgggtgtggaggtgccgctccgagcgctgttcgaagcgccgacggtggcccgtctggccacccagctcggcgacgccggagcggcgcggccg

SEQ ID NO: 16 - DNA, 1563 bp, Chondromyces crocatus
atggagaggcccaccccggtgtcggggcgggccgctggatcacttggcgccgctgcagccgttcttggcgctgcagccgcccttctcgccgtccttggcgccgctgcagccgtccttggcgctgcagccgcccttctcgccgtccttggcgccgctgcagccagccttctcgccgccctcggctgcgtcggccgccggcgcgtcggcggcggcaggggcttccggcgactccgccggctgagccgagccaccacatccggcgagccctgccacgaggcccgagatcgccgtgaacgccaacgtcttgcccatcttcgtcgccatcgatacctccaaggagtgttctctcaccggacccgcgctcatgccgtagcggcgctgcgcagggcggaccggatgtcgcgccctccacccacgggcgtgcgctccatcgagcgcgccgattcaagcaccttcggcccctgcttgaccaccgttccggacgccacgtcggtcgacgaaagcaggggctttccggtccgggccaggtcctgcggggtctcgatcccgggggggggctccttgacgcggtgcgtgctccaggtcgtccgggggtggactggtgggctggctttcctcgtgggcctggtcgcctgcggcgcggcgacgcctggcgcccagcttcctggcgacacgcccctggccgagatcgcggccgagctggaggcgctgggggagccgttgcctccacgcagccgcaccacgttccgcgagatcctcgccgacacgttgagcaaggacgcctatctttgccggccctcggcgcgggaggtgctgctcggcgacgcgcccgagggggagcggcacatcgtgggcatgatgccgcactacgggctgttcatggggccgatgagctacctgatccgccgccgagcgcgcgcctgggaggtcgaggtccgcatcgccgtgacgcccccctcggcctcgcggctggagctccccgactgcgggctccaggaggaagcaggggcccgtgacgcgatgctggtgtgtcacgggacgccgtacgcgcgatcgggatcgaccgacgcttgcccgggctcgggtgagttcgcggcgcgcgccacgccggaggtgatcgccgccttgctcgcgcgctggtcgcgtgaagccgagcggtactggaaccgtgacgccgccgcattcgggctgccgatcacctacgacttcgagttcgtgctggccagcgaggcgagcgcgcgggggctccgcgtcgatctcgacgtgccgctgtcggccacctgcggccgtactccctatttttcggccatgcgcagcggctggtcgctgccggtggtagcgcacgaggtcggccatgtcatgggcctgctcgacgagtacgaggcgctctcgggcatcgtcgccttctacccgaagactccgttcgctggcgccgagatcagccgcatgggcctctcgatgaaggaacacacgcgcgtgcttccgttgcatcattacctcgtgctccggcgttacttctgcagcgatccgggcaccgtggatccttacgctcacgtgttccgc

SEQ ID NO: 17 - protein, 521 aa, Chondromyces crocatus
MERPTPVSGRAAGSLGAAAAVLGAAAALLAVLGAAAAVLGAAAALLAVLGAAAASLLAALGCVGRRRVGGGRGFRRLRRLSRATTSGEPCHEARDRRERQRLAHLRRHRYLQGVFSHRTRAHAVAALRRADRMSRPPPTGVRSIERADSSTFGPCLTTVPDATSVDESRGFPVRARSCGVSIPGGGSLTRCVLQVVRGWTGGLAFLVGLVACGAATPGAQLPGDTPLAEIAAELEALGEPLPPRSRTTFREILADTLSKDAYLCRPSAREVLLGDAPEGERHIVGMMPHYGLFMGPMSYLIRRRARAWEVEVRIAVTPPSASRLELPDCGLQEEAGARDAMLVCHGTPYARSGSTDACPGSGEFAARATPEVIAALLARWSREAERYWNRDAAAFGLPITYDFEFVLASEASARGLRVDLDVPLSATCGRTPYFSAMRSGWSLPVVAHEVGHVMGLLDEYEALSGIVAFYPKTPFAGAEISRMGLSMKEHTRVLPLHHYLVLRRYFCSDPGTVDPYAHVFR

SEQ ID NO: 18 - DNA, 1128 bp, Chondromyces crocatus
gtgccgccctctcgctccgccgcgccccatgccgctctcgccgacggcgcgcctcgccaccccttccgtcctcgcatcgtgctgcgttcgttgatcatgcgcctcgctgaccgtgcgccttcgtgtctgctcgtggtgctcgccgtggggagcgcgctcgtggggtgtcagtgtgctccaggcgtggagttcggtgaggacgcgggcgccgacgccgtggacgcggcggacgtggacgccgcggagcgcgaggtcgcgccgctcgacgatggtggcttcgccgaggagctgctgccgccgccgccaccgccggagcgccgctgcccgccggagatggtccgcgtcgcccgtagcttctgcgtcgatcgcttcgaggcgtcgctggtggacgtcgagacgggctcggcgctctcgccctactaccctccctcgcggcgtcaggcgacctcgatcgagaagctctggcagaagcagcgcctggagatgggcggtgaggaggcgcaggccatggcgttgccgttgctccctgcgttccagcggcagcgcgacgtggaggcgcgcgccgtgtcgcgcaaaggggtggtcccgcagggctacaccagcggagagaaggcggagctcgcctgcaagaacgcgggcaagcggctgtgctcgctggtggagtggaggacggcctgcatgggggaggagcagctccagttcccgtacggaccgaagtaccagggcgggaagtgcaacgtcttccgggagacgcacccggcgctggtgctgcacgacgacatgagccgcgggcacagcgatccgcgcctgaaccaggtcaagcacaagggacgtccgctcctgcggcgcacgggggagacgtcgacctgcatgagcacctgggaggacgacgccatcgccgacatggtcggcaacctcgacgagtgggtcgccgacgaggccggcacgttcgtcggtggtttctacgcgcggtcgacgcgcgatggctgcatgtccacggtgaccgcccacaccttcgattacttcgactactcgacggggatccgctgctgcatggatctgccggaggcgcggcccagcgtgccctcgcgcgctggatcgagccgtgatgcagcggacgaggcccgc

SEQ ID NO: 19 - protein, 376 aa, Chondromyces crocatus
VPPSRSAAPHAALADGAPRHPFRPRIVLRSLIMRLADRAPSCLLVVLAVGSALVGCQCAPGVEFGEDAGADAVDAADVDAAEREVAPLDDGGFAEELLPPPPPPERRCPPEMVRVARSFCVDRFEASLVDVETGSALSPYYPPSRRQATSIEKLWQKQRLEMGGEEAQAMALPLLPAFQRQRDVEARAVSRKGVVPQGYTSGEKAELACKNAGKRLCSLVEWRTACMGEEQLQFPYGPKYQGGKCNVFRETHPALVLHDDMSRGHSDPRLNQVKHKGRPLLRRTGETSTCMSTWEDDAIADMVGNLDEWVADEAGTFVGGFYARSTRDGCMSTVTAHTFDYFDYSTGIRCCMDLPEARPSVPSRAGSSRDAADEAR

SEQ ID NO: 20 - DNA, 2811 bp, Chondromyces crocatus
gtgagggcgaccctgggagagagcggaccgggagtcgcgtggatcctgtacgctcggccgcgacgcggtctgtcgcgcttccctggagtctcggccatgcgctcttgcccgcacgtgcttccgttggtgttctctgcgctcctgctcccggtgctcgcggaggcctcaccgggtcacgctgcgccgtccgcgtcttccccctcgctcgctgcgccgcgcgcgacactcgcccacgcggtgccaccggcatcgctgccagcgcccgcgcgcctggccgcggcgctctcccgcttgccgcgccacgcctcgctggagggcgcgctgccgctttcgttcacgccccgcgtgagcgccggtgggcgacgggggatcacgccggccgcgagcgaggtgccggtgctggttcgcttcgcagcgcggcccacgcccgccgagatcgacgcgctccgggccgctggcgccgtgcctcggctgcgcgccgacggatctccccgcggacagggggacgtgatcgtcgcgcgcatgtccctcgacacggtcgcgcgcgtcgcggcgctccctttcgtgcgctcgctccgccccgacggcgcccctttccgctcgcctcggccgatcgatgcgaccggggccgagatccaggccagcgacgcctggggcgccgggatgaagcccggtggggtgatccagggggtgaccggccgtggcgtcgtcgtctgcgacgtcgacagcggcgtcgaccccttccacccgctgttcttccgcgccgacggcggctacttcgactgggtcgacgtcgacggagacggccgcttctccccggggatcgacggggtggatcgcgatggcgacggggtgccggagatcctgcgcacgctgaacagcgtgatcacgaactactacgacgatgagccgctcctgggctcggagagcgaggcgttcgcggcggggatggactggctctacgccgatctcgacggcagcggggcgcgcgagttcggcacggcctccggcttcaccgaggaggatcccacgtacggcgagccgctgtacgtcgtcgacgacgtgaaccgcaacggtaccctcgacgtcggcgagcggatcgtggcgctgggaagctccaagatccggatgatcgtgaacggtagcgagcgcttccagcgtggcgtgaacctgatccgggcgcggcgtgacgagagcatcgcgcacggcaccggctcggccggggtgatcctggggggggccatcggcctcacgcgcttcacggggatcgcccccgacgcggagctgatcatggcgaccgcgagcgacgccgtgggtgagttcgagctgaccgatctgtgcatcgaggaaggcgcccgggtggtgctccacgagtacgcgccctggatcggtcagccgctcgatggctccagcgcgctggagcagctcatcgacgagaccgcgcgccagtgggtcgcgcacatcaatccggccgggaacctctcgggaggtgacaagctctccaaacgcacgctcgctgccggagaggcggtcgtcgcgtcgctgcacgtcccggaagacagcccctacggggcgttcggcgcgatgtacacctcctggctctggcgcgcgccggatcgcaacctgacgttcaagctcaccgatccgcagggcgtctcgaaggatctcacgctgaacgcggacgatccgagcgtcccgtacatctacgagagctgggggagcgaaggcggcgtcatggtgtacgccgcgcgcgagaactcgccgcgagggaccgcgcgtctcgacgtgttcctggtggacacgggcctcgtggcgtcccccctccccgcgggggactggacgatggagatcgtcgaccccggtcctgtggatggttcgtccatcgagctgatcggctacgtgatggatgaactctcggggtgggggcacggcatccatttcccggatcacgtgagcgaggatcacctcatcggctacccgggcaccgcggacttcgggctcgccgtggccgcgtacacggggctgggccgctggggcggtgagcccggcgtgcgcgcctcgtactcggggcgggggtggcgcctcgacggcgcgcccttgctgtggatcagcgcgcccgacgacccgatcaccagcggctaccgggaaggtgaggaggcgcgctacagcgtcttcggtgggacctcggccgcgagcccgcacgtcgctggcgccgccgcgctcctcttcgaggcagaccctgcgcgcaccgggctcgacgtgcgcgaggccatccgcgccggcgccctcgtcgacgacatcgtgggcagcgcgccgagcacggactacgggcacggcaagctgcgcatctacaagagcctccacggcgtcgagccgccggccggtgcgcccccgcggctcgcgccgatcgaggcgcgggtcgaggtgggggtgaagaccgcgctctccctcgacgcgtccgagcccgccgacgtgctgaacacgctcttcttcgacgtcgatcgggactacgacgggatctgggaggagtcgctcgtcggctcctccctgatcgtcctcttctacgagaccggcgtccacaccctcaagctgcgggtgcgggacgccaccgggcgctcggggaccgcgctcgcgcgcatcgaggtcgtcccccgtggcgcggtgccgaaggcccagacgctcgtcgctggtggcggctgggactgcgcgctgggcaaggccgagggcacgagcgccgggtggctcgtggccgcggggctcgcgacgatcggcgttcggcgccggcgaacatctcgcctgtcatcgcaccgc

SEQ ID NO: 21 - protein, 937 aa, Chondromyces crocatus
VRATLGESGPGVAWILYARPRRGLSRFPGVSAMRSCPHVLPLVFSALLLPVLAEASPGHAAPSASSPSLAAPRATLAHAVPPASLPAPARLAAALSRLPRHASLEGALPLSFTPRVSAGGRRGITPAASEVPVLVRFAARPTPAEIDALRAAGAVPRLRADGSPRGQGDVIVARMSLDTVARVAALPFVRSLRPDGAPFRSPRPIDATGAEIQASDAWGAGMKPGGVIQGVTGRGVVVCDVDSGVDPFHPLFFRADGGYFDWVDVDGDGRFSPGIDGVDRDGDGVPEILRTLNSVITNYYDDEPLLGSESEAFAAGMDWLYADLDGSGAREFGTASGFTEEDPTYGEPLYVVDDVNRNGTLDVGERIVALGSSKIRMIVNGSERFQRGVNLIRARRDESIAHGTGSAGVILGGAIGLTRFTGIAPDAELIMATASDAVGEFELTDLCIEEGARVVLHEYAPWIGQPLDGSSALEQLIDETARQWVAHINPAGNLSGGDKLSKRTLAAGEAVVASLHVPEDSPYGAFGAMYTSWLWRAPDRNLTFKLTDPQGVSKDLTLNADDPSVPYIYESWGSEGGVMVYAARENSPRGTARLDVFLVDTGLVASPLPAGDWTMEIVDPGPVDGSSIELIGYVMDELSGWGHGIHFPDHVSEDHLIGYPGTADFGLAVAAYTGLGRWGGEPGVRASYSGRGWRLDGAPLLWISAPDDPITSGYREGEEARYSVFGGTSAASPHVAGAAALLFEADPARTGLDVREAIRAGALVDDIVGSAPSTDYGHGKLRIYKSLHGVEPPAGAPPRLAPIEARVEVGVKTALSLDASEPADVLNTLFFDVDRDYDGIWEESLVGSSLIVLFYETGVHTLKLRVRDATGRSGTALARIEVVPRGAVPKAQTLVAGGGWDCALGKAEGTSAGWLVAAGLATIGVRRRRTSRLSSHR

SEQ ID NO: 22 - DNA, 1659 bp, Chondromyces crocatus
atgaacatcatcgagctcatcaccgaggaagcccgcgaggcacgcgaccgcattctcattgccgcgagcgtcgcaggcgtggccaacgcggcgacggtggcgctggccaacgccatcgtccagagaacggacggcggcgccacgctcgccgactgcggtctcttcgtcgggctgatcgccgtctacgtcctctgctccaggtacacgtgccatcgcgtctccgggacgatcgaggacgcgctccacaccatcaaggtgaggatcctcgagaaggtcgagcgcgcgagctacgagagcatcgagcggatcgggacggccgagatctacgaccggatcacgaccaacgtctcgaagatctccggctcggcgacgctgatcgccaacctgctccagtcgctcttcatgtccgtcgcggcagggctctacgtcgcctccctctcgctgccggccttcacgttgctcgtcgtcctcctcggcggcgggatcacgctcttctacctcaggtcccaggacgtgaccggggatctccagcgcgcggccgccatccggctggggttcttcgaccggctcaccgatctcttcaagggcttcaaggaggtcaagctgagccgccggcgcgggcaggagcttcgcgaggacatccgcgacacctcgggcgcgctgcggaacgtggcgacgcggtcgggcaacgcctttcacgatcactggctcttcgctcagtgcaacctctacgtggcgctcgccgccatcatcttcgtgctgccccagcacgtcgaggtggccgcgacgacggagcgattgctcctcggaggcgtcctcttcgcctgggggcccatcgtcacctgcatcgcgggcttccccgcctacgtcgagtcgaacgtcgccctgggcaacatcgacgcgctggagcagaagctcgacgccgcggtggtggagtgtgagggcgacgacccctgggaggggaagctgacggagggcatcgtggtcaaggacctcgcgtacgcctatgcgtcggaggatgctcgggaggccttccacatcggcccgatcgatctcgacctcgccgcaggagaaatcgtcttcatcgtcggcggtaatggcagcggcaagtcgaccttcctcaaggtgctcaccggcctctacccaccgagcggcggcacgctgagcgtggacgggttcgaggtcacgccgggtcgggccgccgcgtaccgcgagttcatcaccgcgatctactcggacttccacatcttctcccggctgtacggcctcctcggcgtgcccgaggcggcggtgcgcccgctgctcgagcagatgcagatcgaggacaagacctccttcgagggcgaccgcttcacccggaccaacctgtcgacggggcagcgaaagcgcctcgcgatgatcgtggccctcctcgaggaccgaccgatctgcatcttcgacgagtgggcggcggaccaggatcccgagttccgtcggtatttctacgaggagcttctcccttccctgaagcgacgaggcaagacggtcatcgccgtgagccacgacgaccgctacttcggctgcgccgaccgcgtggtgaccatggagtacgggaaggtccgctcgatcgggccgggccaggagcctcagaacagcccaggcgaaacccacggggccaca

SEQ ID NO: 23 - protein, 553 aa, Chondromyces crocatus
MNIIELITEEAREARDRILIAASVAGVANAATVALANAIVQRTDGGATLADCGLFVGLIAVYVLCSRYTCHRVSGTIEDALHTIKVRILEKVERASYESIERIGTAEIYDRITTNVSKISGSATLIANLLQSLFMSVAAGLYVASLSLPAFTLLVVLLGGGITLFYLRSQDVTGDLQRAAAIRLGFFDRLTDLFKGFKEVKLSRRRGQELREDIRDTSGALRNVATRSGNAFHDHWLFAQCNLYVALAAIIFVLPQHVEVAATTERLLLGGVLFAWGPIVTCIAGFPAYVESNVALGNIDALEQKLDAAVVECEGDDPWEGKLTEGIVVKDLAYAYASEDAREAFHIGPIDLDLAAGEIVFIVGGNGSGKSTFLKVLTGLYPPSGGTLSVDGFEVTPGRAAAYREFITAIYSDFHIFSRLYGLLGVPEAAVRPLLEQMQIEDKTSFEGDRFTRTNLSTGQRKRLAMIVALLEDRPICIFDEWAADQDPEFRRYFYEELLPSLKRRGKTVIAVSHDDRYFGCADRVVTMEYGKVRSIGPGQEPQNSPGETHGAT

SEQ ID NO: 24 - DNA, 1680 bp, Chondromyces crocatus
atgaccctcatcgacctcatcacccaggaagccgcgccggagcgccgcaggatcctcatcgcggccggcatcagcggcgtcgccaacacgctcgtcatgggcatggtcaaccagatcacccagacggacgtcgccgagacgagcgcccggacgttcgccatgttcggcctcgccgtcgccatgtacgtggtcggcgccaggaacatctaccaccgcatgaccacggtcctggagagcgcgctgcaccgggtcaagacgaggatcgtcgccaaggtcgcgcaggccgacatggagaagctcgagcggatcgggacggcggagatctacgaccggatcaccgacaacgtcgcggtggtctcggagtcggcggggcggctcgcgttcttcctgcagtcggtgtgcatcatcgtcgcgagcacgctctacctcgcctccctgtcgctcccggcgttctgcgcgatctcgctcctgatcgtgggtgggttcatgctctacgccggcaagaatcgagagatcggggagtatttccaccgcgccgcgctgacgcgcataacgttcttcaatcagctcaccgacctcctccagggcttcaaggaggtgaagttcagccggcgccgcggacgcgagctccgggaagacctcgtgcagacgtcggcgaccttgcgggacgacatgaaggtcgccagcgggctgctcgacgacaacttcatcttcgccgcctgcatcctcttcgccgcgctggggaccatcgtcttcctcatgcccttgcagatcgagatcgcgaagaagacccagcagatgctcatcgccggtgtcctgttcgcctgggggccactcagcgggtgcatgggggggatcccggcgtacatccgatcgaacgtcgcgctctccgcgatcgatgagctggagcggaggctcgacgacgcgctcgaagcggcaccaggagacgaggcgagcgatccgtggggtggacgcctcacgaaggccatcgaggtcgaccagctcgagtacacgtacgcttcggacgacgggagggaggccttccgcatcggcccgatgagcctgaccatccaggcaggcgagatcctcttcatcgtcggcggtaacgggagcggcaagtcgacgttcctgaagaacctcaccggcctgtaccggccggagctgggcgctctgatcgcggatggcacgccggtgaccgaggcgaacgtcgccgcctaccgggagctgttctcggcgatctactccgacttccacctcttctcgaagctctacggtctgccgaccgtggacgccgcgaaggtgcgctcgttgctcgagcagatgcacctccagaagaagaccgccttcgagaaggaccacttcacccggcgtgacctgtcgacgggtcagcggaagcgcctggcgatgatcgtgagcttgctcgaagaccgacccatctgcgtcttcgacgagtgggcggcggatcaggacccggagttccgccgctacttctacgaggagcttctgcctgccctcaggcgacagggaaagaccatcctcgcggtgagccacgacgaccgctacttccactgtgcggatcgggtcatcacgctggagtacggcaagatccgatcgatcgaaccaggctcggcgcgtggcattccccccgctccagggaaggaccggcggaggtccgatgcg

SEQ ID NO: 25 - protein, 560 aa, Chondromyces crocatus
MTLIDLITQEAAPERRRILIAAGISGVANTLVMGMVNQITQTDVAETSARTFAMFGLAVAMYVVGARNIYHRMTTVLESALHRVKTRIVAKVAQADMEKLERIGTAEIYDRITDNVAVVSESAGRLAFFLQSVCIIVASTLYLASLSLPAFCAISLLIVGGFMLYAGKNREIGEYFHRAALTRITFFNQLTDLLQGFKEVKFSRRRGRELREDLVQTSATLRDDMKVASGLLDDNFIFAACILFAALGTIVFLMPLQIEIAKKTQQMLIAGVLFAWGPLSGCMGGIPAYIRSNVALSAIDELERRLDDALEAAPGDEASDPWGGRLTKAIEVDQLEYTYASDDGREAFRIGPMSLTIQAGEILFIVGGNGSGKSTFLKNLTGLYRPELGALIADGTPVTEANVAAYRELFSAIYSDFHLFSKLYGLPTVDAAKVRSLLEQMHLQKKTAFEKDHFTRRDLSTGQRKRLAMIVSLLEDRPICVFDEWAADQDPEFRRYFYEELLPALRRQGKTILAVSHDDRYFHCADRVITLEYGKIRSIEPGSARGIPPAPGKDRRRSDA

SEQ ID NO: 26 - DNA, 9078 bp, Chondromyces crocatus
atgaccagcatcgcaggtccggctcgcccgaagccggaagacgccacgacgaatcgggacaccacgacctccatctcggccgagttcacggtcgagccggtgcgtcctgcggcgtcgagcgtgagcgggatggctggcctggcggaggctgcgcggcgcgagggcagggtgcctgcgtcgtcggggcagcggcggctctggttggtcgagcggctcgccgccgagcgcacgctgtacaacgtgcacctctgcgtgcgcatggaggggccgctcgatccgtcatggctccgacagagcgtggccatgctcttcgagcgacacgaggtgcttcgcatgcggctccacgaggtcgacggggatgtcctcgggatcgtcagccccccgggtgaggtggagctgcccctcgtcgaccttcgccaggtgccacccgaagcccgagggcagcggttctctcaggtctcgatggatcacagcctcacgcccctggatctcggtgtcgggcctgtcgtgcggatgacgctggtggcgctgaaggacgacgagcacgtcctcctggtcacgcagcaccacgccgtcaccgacgggcggtcgctcatgctcctgccggcggagctcttcgccttctaccgcgcgctctgcgatggaacgtcgcctcgtctgcccaccctgcccatcacctacgcggacttcgtggtctgggaggcccaggcgcggcagtcgccgcacttcgccgcgcatctggcgtggtggcagacccgcctctcgaacctccccgagctggagcttcccttcggtcgcaaggtcgaagcgcccacgtacaccggggacttcgtgacgttcgtgtacccgctcgtgctcacctccgggctggagtcgatcgcggcgcggcacgggagcaccctgttcagggtcctggtggcggcctgggctgccttgctccaccgctacaccggtcagaccgacttccccatcggcacggtcacggccatgcgcagggacccccagctgcatggcctcctcgggtacttcgcccacaccctcgtcttgcgctgcgagctggaggccgaccagacgttcctcgatctcgtggcccggatcgacggcgtggtgcgggaagcgctggcgcacgcagaggtgcctttcgacgacatcgtccgtgccgtgggggcctcgcgtcgaggacacctcaacccgctggtccagtcctccttcgtgctcgagaactactcgttccacgctcacgaagccgccgatcagcggtggacgccgtacttcgaggagatcgacgcgggcgtgaagggaggggcgaaattcgacgtctccatggccctctacgtgacgcccgagggcttgaaggggaagctcgagttcgcgacggatctgttcgagcgcgccgccatggaacggctggtgagccacttcgaggcgttgctcctcgatgtggtcacccacccggcccggcgtttgtcggatctggcgttgctgtccggcgtggagcgacgccagctgctggtcgactggaacgagaccgcgagggacttccgtcgagcgacgtgcatccacgagctgttcatggaacaggcctcgcggacaccggaagccgtcgcggtgcacttcgaggaggagcagctcacgtacggcgagctggacgcccgctccaaccagctcgcgcaccacctgcgcgcgctgggcgtggggcccgaggtgctggtgggcctgtgcgtggagcggtccctcgacatggtcgtggggcttctgggcatcgcgaaagccggcggcgctcacgtgccgctggatccggcgtatccgccggagcggctggcgttcatgctggaggacgcgcgcgcgagcgtcctgctcacgcaagcgccgctggtcgagcggctcccggcgatctcggcgcgggtcgtgtgcttcgacgcggatgctcctgcgctggctgcatggccacgctcgaccccggaggtcgtcgtcacgtcggacaacctggcctacgtcatctacacgtcgggctcgacgggcacaccgaagggcgtgatgtgcacgcaccgcgggctcgtcaacctcgtggaccacgaggccgagctcctcgagattggtcaggggaccccggtcctgcagttcgcttcgatctcgttcgacccctccctctcacagctcctcggggccctgagccggggcggaatcgtggttctcgcgtcggccgatcaacggcgctccagcgccgcgctgacagggctgctgcgggcccggggcgtggaggtcgcccacctgccgccgagcgcgctttcgctcctcgacgagagcgatcccctggcgctccgtgtgctgatggtgggcggtgaggtctgccccgtcggtgctgccacggtctgggcccgtgggcgccgtttcatcaactcctacggtccgacggagacgacgatcacggtgtcgtactgggaagggaagccgtcgcccggcgcctccgttccgctcggcaagccgaacgccaacacgcaggtttacgtgctctctcctgcgatgcaggtgctcccgatcggggtgccgggggagctcttcatcgccggcgctggcgtctcgcgtggctacctgaagcgaccgggcctcaccgccgcacgcttcctccctgatcctttcgggccagccgggggcaggatgtaccgcaccggcgacctttgccgctggcgggaggatggcaacctcgagttcctgggccgtatcgaccaccaggtgaagatccggggcttccggatcgagctgggagagatcgagtcggtgctggagcagcaccccgcggtgcgcgcttgcgtggtcatggcgcgcgaggacgagcccggcaaccagcgcctggtcgcgtacgtggtgcctgcggcggacgaggagggctcgatcgctgatctgcgtgcgcacctcaaggcgaagctgccggaccacatgatcccgtcagcgttcgtcgccttgcccgtcctcccgctcagcgcgaacggcaaggtggatcgcaaggccctcccggcccccgacggtcgcgccgaggatcaccgcgcattcgtggcgccacgcacgccggtcgaggagctgctcgccgagatctggagcggcctgctcggcgtcgggcggatcggggggcaggacgatttcttcgagctggggggacactcgctcctggcgacgcaattgatcgcgcgcctccgcgccgccttcggcgtcgagctgcccatgcgcggcgtgttcgaggcgcggacgctggcgaagctcgccacggagatcgaggcggcgcggcaaggggggcaatcgcacgacgagctgcccctcgtgcccacggagcgggagcgcgcggtccccctgtcgttcgcccaggagcggctgtggttcctggaccggctcgaacccgacagtcctttttacaacatcccggtggtggtgcgtctcgcggggaacctggacgtgcacgccctcgagcggagcctcggcgagatcgtgcgccggcacgaggcgctgcggacgatctttccggcggacgatgggcaggcccgccaggtggtgacgacgccctccgactggcgcttgcccctcgtcgatgtgcctgcgggcgagctgcgtcggcgcatcgaagcggaagctcgggctccgttccgcctcgcggagggaccgctgttccggggcacgctgctgcggctgtcggagcgagagcacgtgctgctcttgacgatgcaccacatcgtcagcgacggctggtcgatgggggtgctcgtgcgtgagctgggcgcgctctacgaagccttctcggcggggaagccctcgtcgttgcctgcgctgcccgtccagtacccggacttcgcgctgtggcagcggcgcgtgctgagcgaggcgcgcctcgatgcgctgctcgcgtactggcaggcgcagctgtcgggcgcgccgccgctcaccttgccgacggacaggcctcggccgcccgtggcatcccatcgggggagcaccgtcacgttccagcttcctcgtgcgatcggcgaggggctgcgcgcgctgggccgcaaggaaggcgcgacgctgttcatgacgctcctgtcggccttcgcggtgatcctcggccggcacgcgaaccagctcgatttctgcgtggggacgcccgtggcggggcggacgcggagagaggtcgaggggatgctcgggtgcttcatcaacaccctggtcctgcgcgccgacctgtccggggatcccagcttccggagactcatgggccgcatccgcgaggtggcgctcgccgcgtatgcccatcaggacgcccccttcgagcggctggtggagcggctgggcgtttcgcggagcctcgggcacagcccggtgttccaggtgatgttcgtcctccagagcgccccggtggacacgtttcgtcttccgggcctggtgatctcgaccgcgcaggagacgacgagcaccgcgaagttcgatctgaccctctccatggaggagggccccgaggggctctccggcgtgttcgagtacgcgacggacctgttcgatgcggcgacggtcgagcggctggccgggcacttcggcgtgctcctgcgcgcggtcgtgcaagacccggacgcgtcgatcgcgacgctgccgctcctgacggaggacgagcggcagcgcgtgctggtgacatggaacgaggggggaacggagccctctcccgtcggctgcctccacacgctgttcatggagcaggcgtcgaggacgccggacgccatcgcggtgcgctgcggtggggagcagctcacgtacgccgagctggatgcccgatccagccgcctcgcacatcacctccggggcctgggcgtgcgcgccgacggcctcgtcgggttgtgtgtcgagcggtccctcgacatggtcgtgggcctcctcgggatcctgaagaccggcggcgcctacgtgccgctggatcctgcgtatccgcaggaccgcctggcgttcatggtgcgggacacgcaggtgcaggtggtggtcacccagtcgcgggtggcgcacgtgctgcccgagagcgaggcgcggctcgtgcggctggacgccgactgggcggagatcgcgcaggcgtccgcggagccgcccgcctccggcgcgacgcctggcaccctggcctatgtcatctacacgtccggctcgacggggacacccaagggcgcgatggtcgagcacggccatgtcgtccggctgttcacggcgacggccgcgtggttccagttcggcgcgcgggacgtgtggacgatgttccactcggtggccttcgacttctccgtctgggagctgtggggtgcgctgctccacggaggccgtgtggtggtcgtgcctcacgcggtgagccgggatcccgaggcgttccacgcgctcgtcgtgcgcgagaaggtgacgatcctcaaccagaccccgtcggcgttccgcgagttcgtccgggtggacgggagcgtctctcatgagacccgtgcggcgctcgcgctgcgccacgtgatcttcggcggggaggcgctcgatgtgggggagctgcggccctggtgggatcggcacgaggacgacgcgcccgtgctggtcaacatgtacgggatcaccgagacgaccgtgcatgtcacccatcggcccctgagccgggcggatctggagcgaccctggtcgagcaccatcgggcgtccgatccccgacctgcaggtgtacgtgctcgatgcggcgcgcaacccggtgcccatcggggtgtccggcgagatgtacgtcggaggagcgggggtctcgcgtggctatctcgggcgcagcgcgctcaccgccgagcgcttcgtcgaggatccattctccgcccggcccggggcgcgtctgtaccggaccggggatctcgcccgctggaacagcgcgggggagctcgagtacctgggccggatcgatcagcaggtgaagatccgggggttccgcatcgagctgggggagatcgaggcggtgctcggggagcaccctgcggtgcgcgcgtgcgtggtcgtggcgcgcgaggacgtccccgggaacaagcgcctggtggcctacgtggtgcccgacgagggcggcgtcccgacggcggcgtaccgtgagcacctgcgggcgaagctgcccgagtacatgatcccggcggccttcgtcgtcctcgacgcgctgccctcgaccccgagcggcaaggtggaccgcagggcgctgcctgcgcccgagcagcgcccggaggacggctgctccttcgtcgcgcctcgcacgcccgtggaggcgctgctcgccgagatctggggcgggctgctcggcatcgagcgcgtcggcgcagaggacgacttcttcgcgctcggcggtcactcgctgctggccacacaagcgatctcgcgcatccgtgccgcgttcggcgtcgatcttcccctgcggacgctgttcgaggcgccgaccgtggcggagctcgcggcgaggatcgacgggatggcgcgcgacgcggcgggcgtgggcgacgcgacggaggaggaccagcccctcgtcccggtggcgcggggcgccgcgctgcccctgtccttcgctcaggagcggctgtggttcctcgacaggctggagccgaactgcgccttctacaacatcgccacggccttccacctcgcggggcccctcgatggggaagcgctcgcgcggagcctccgggagatcgtgcgccggcacgaggcgctgcgaacgacgttccctgcccgtgaaggtcaagctcaccaggtgatcggcgaggccgcgcgctggaccctgacgcacgcagacgtccagccgtcggagtggcgccgccgcatcgaggaggaggcccgtgcgcccttcgatctcgcggcgggcccgctcttccgggcgacgctcctgcgcgtgtcggacgtggagcacgtgctgctcctgacgatgcaccacatcgtcagcgatggctggtcgatgggcaccctcgcgcgtgagctggaagccctctacggtgccttcgccgccgggcggtcctcccccctggccgagctgccggtccaggtggccgaccacgccgtctggcagcggagccggctacgagggagaggcttcgaggcgcacctggcctactggcaggccaagctcgccggcgcgcagcctctcgtcctgccgacggatcgcccgaggccgccggccgcgtcgcaccagggtcgtctgctgaccttccagctcccccgagcgctcgcggtcgagcttcgcgcgctgagccgcaaggagggggcgacgctgttcatgaccttgctctcggccttcgcggtgctcctcgcgcgccacgcgaaccaggtcgacttctgcatcgggacgccgatcgccacgcggaaccgggaggcgctcgaagggctgatcggtctcttcgtcgacacgctcgtcctgcgggccgacctctcgggtgatccgaccttccgtgcgctcctcggacgcatgcgggacgaggcgctggcgagccacgcccaccaggaggtccccttcgagcgcatcgccgacaggctgggggtggcgcggagcctcggccagagcccggtgttccaggtgatgttcgcgctgcagaacgcgccgatggacgggctccgtctgccaggggtcgaggtgacctccgaggaggtggagacggggacctcgaagttcgatctctcgctctcgatgcaggagcatgccgaggggctcgtcggcgtgttcgaggtcgcgacggacctgttcgacgtctcgaccgtcgagcgcctcatcggtcagttcggcgtcctcttgcgcgcggtggtgcgtgacccggaggtgccagtgtccacgctgcccctcctgacggaggccgagcgccaccagtcgctcgtgacgtggaacgacacggcgacggctgccccgcaggatcggtgcgttcacgcgctgttcatggagcgagcggcgaggacacctggcgccctcgcggtgatccacggcgaccggcagctcacctacgccgagctcgatgctcgctccagccagctcgcgcaccacctgcgagcgcggggagtcggccccgggacgctggtggcgctctgcgtcggccgctccgtcgatctgatcgtgggcgcgctcggcgcgctgaaagcagggggagcctacgtccctctggacccggcccatccagcggagcggctggcgttcatgctggaggacacgggcgcgaccgtgctgctgacccaggcagccctcgtggcacggctccccccgcacggcgcgcaggtcgtgctcctcgacgccgacgacgcgaccctcgacgcgtggcccgacgtggcgccgcccctgcgtacgacgtcggaggatctcgcttacgtcatctacacctcgggctcgacgggccggccgaagggcgtcctgctctcgcacgggggcctcgtgaacctctgcacgtggcacgtgggggcgtaccagctctctccagaagatcgcacgacgctgatcgcagcgccggggttcgacgcctcggtgtgggagatctggccagcgctgatcgcgggcgcctcgctgctgatcgtggacgacgagatccgcctgtcgccagccgcgctggcggacttcctcgtcacgcgcgaggtgacggtgaccttcctcccgacaccgctcgcggaggcgttgctgaccctcccctgggccacgggtggcgcgctgcgcgcggtgctgacgggcggagacgtcctgcggcgaaccccacccgcggcgctgcccttcgcgctcgtgaaccattacggaccgacggagtgcaccgtcgtggcgacggcggccgtggtcgtgccgggggggcagggggcgccaccgatcgggaagccgatcgcgaacgcccgggtgtacgtgctggatgcgcgcggcgcgcccgtgcccgtcggtgtccctggcgagctgtacatcggcggcgccggcctcgcccagggctacgcgaaccggccggcgctgacggcagagcggttcgtccccgaccccttcggcgacaccccggggcgtctctatcgcacgggggatctcgtgcggtggctgcccgacgggagcctcgcgttcctcggcgcatcgacgaccagg

SEQ ID NO: 27 - protein, 3026 aa, Chondromyces crocatus
MTSIAGPARPKPEDATTNRDTTTSISAEFTVEPVRPAASSVSGMAGLAEAARREGRVPASSGQRRLWLVERLAAERTLYNVHLCVRMEGPLDPSWLRQSVAMLFERHEVLRMRLHEVDGDVLGIVSPPGEVELPLVDLRQVPPEARGQRFSQVSMDHSLTPLDLGVGPVVRMTLVALKDDEHVLLVTQHHAVTDGRSLMLLPAELFAFYRALCDGTSPRLPTLPITYADFVVWEAQARQSPHFAAHLAWWQTRLSNLPELELPFGRKVEAPTYTGDFVTFVYPLVLTSGLESIAARHGSTLFRVLVAAWAALLHRYTGQTDFPIGTVTAMRRDPQLHGLLGYFAHTLVLRCELEADQTFLDLVARIDGVVREALAHAEVPFDDIVRAVGASRRGHLNPLVQSSFVLENYSFHAHEAADQRWTPYFEEIDAGVKGGAKFDVSMALYVTPEGLKGKLEFATDLFERAAMERLVSHFEALLLDVVTHPARRLSDLALLSGVERRQLLVDWNETARDFRRATCIHELFMEQASRTPEAVAVHFEEEQLTYGELDARSNQLAHHLRALGVGPEVLVGLCVERSLDMVVGLLGIAKAGGAHVPLDPAYPPERLAFMLEDARASVLLTQAPLVERLPAISARVVCFDADAPALAAWPRSTPEVVVTSDNLAYVIYTSGSTGTPKGVMCTHRGLVNLVDHEAELLEIGQGTPVLQFASISFDPSLSQLLGALSRGGIVVLASADQRRSSAALTGLLRARGVEVAHLPPSALSLLDESDPLALRVLMVGGEVCPVGAATVWARGRRFINSYGPTETTITVSYWEGKPSPGASVPLGKPNANTQVYVLSPAMQVLPIGVPGELFIAGAGVSRGYLKRPGLTAARFLPDPFGPAGGRMYRTGDLCRWREDGNLEFLGRIDHQVKIRGFRIELGEIESVLEQHPAVRACVVMAREDEPGNQRLVAYVVPAADEEGSIADLRAHLKAKLPDHMIPSAFVALPVLPLSANGKVDRKALPAPDGRAEDHRAFVAPRTPVEELLAEIWSGLLGVGRIGGQDDFFELGGHSLLATQLIARLRAAFGVELPMRGVFEARTLAKLATEIEAARQGGQSHDELPLVPTERERAVPLSFAQERLWFLDRLEPDSPFYNIPVVVRLAGNLDVHALERSLGEIVRRHEALRTIFPADDGQARQVVTTPSDWRLPLVDVPAGELRRRIEAEARAPFRLAEGPLFRGTLLRLSEREHVLLLTMHHIVSDGWSMGVLVRELGALYEAFSAGKPSSLPALPVQYPDFALWQRRVLSEARLDALLAYWQAQLSGAPPLTLPTDRPRPPVASHRGSTVTFQLPRAIGEGLRALGRKEGATLFMTLLSAFAVILGRHANQLDFCVGTPVAGRTRREVEGMLGCFINTLVLRADLSGDPSFRRLMGRIREVALAAYAHQDAPFERLVERLGVSRSLGHSPVFQVMFVLQSAPVDTFRLPGLVISTAQETTSTAKFDLTLSMEEGPEGLSGVFEYATDLFDAATVERLAGHFGVLLRAVVQDPDASIATLPLLTEDERQRVLVTWNEGGTEPSPVGCLHTLFMEQASRTPDAIAVRCGGEQLTYAELDARSSRLAHHLRGLGVRADGLVGLCVERSLDMVVGLLGILKTGGAYVPLDPAYPQDRLAFMVRDTQVQVVVTQSRVAHVLPESEARLVRLDADWAEIAQASAEPPASGATPGTLAYVIYTSGSTGTPKGAMVEHGHVVRLFTATAAWFQFGARDVWTMFHSVAFDFSVWELWGALLHGGRVVVVPHAVSRDPEAFHALVVREKVTILNQTPSAFREFVRVDGSVSHETRAALALRHVIFGGEALDVGELRPWWDRHEDDAPVLVNMYGITETTVHVTHRPLSRADLERPWSSTIGRPIPDLQVYVLDAARNPVPIGVSGEMYVGGAGVSRGYLGRSALTAERFVEDPFSARPGARLYRTGDLARWNSAGELEYLGRIDQQVKIRGFRIELGEIEAVLGEHPAVRACVVVAREDVPGNKRLVAYVVPDEGGVPTAAYREHLRAKLPEYMIPAAFVVLDALPSTPSGKVDRRALPAPEQRPEDGCSFVAPRTPVEALLAEIWGGLLGIERVGAEDDFFALGGHSLLATQAISRIRAAFGVDLPLRTLFEAPTVAELAARIDGMARDAAGVGDATEEDQPLVPVARGAALPLSFAQERLWFLDRLEPNCAFYNIATAFHLAGPLDGEALARSLREIVRRHEALRTTFPAREGQAHQVIGEAARWTLTHADVQPSEWRRRIEEEARAPFDLAAGPLFRATLLRVSDVEHVLLLTMHHIVSDGWSMGTLARELEALYGAFAAGRSSPLAELPVQVADHAVWQRSRLRGRGFEAHLAYWQAKLAGAQPLVLPTDRPRPPAASHQGRLLTFQLPRALAVELRALSRKEGATLFMTLLSAFAVLLARHANQVDFCIGTPIATRNREALEGLIGLFVDTLVLRADLSGDPTFRALLGRMRDEALASHAHQEVPFERIADRLGVARSLGQSPVFQVMFALQNAPMDGLRLPGVEVTSEEVETGTSKFDLSLSMQEHAEGLVGVFEVATDLFDVSTVERLIGQFGVLLRAVVRDPEVPVSTLPLLTEAERHQSLVTWNDTATAAPQDRCVHALFMERAARTPGALAVIHGDRQLTYAELDARSSQLAHHLRARGVGPGTLVALCVGRSVDLIVGALGALKAGGAYVPLDPAHPAERLAFMLEDTGATVLLTQAALVARLPPHGAQVVLLDADDATLDAWPDVAPPLRTTSEDLAYVIYTSGSTGRPKGVLLSHGGLVNLCTWHVGAYQLSPEDRTTLIAAPGFDASVWEIWPALIAGASLLIVDDEIRLSPAALADFLVTREVTVTFLPTPLAEALLTLPWATGGALRAVLTGGDVLRRTPPAALPFALVNHYGPTECTVVATAAVVVPGGQGAPPIGKPIANARVYVLDARGAPVPVGVPGELYIGGAGLAQGYANRPALTAERFVPDPFGDTPGRLYRTGDLVRWLPDGSLAFLGASTTR

SEQ ID NO: 28 - DNA, 15228 bp, Chondromyces crocatus
atgcgccctcggaccggcggctggtcgcgtacgtggtccccgccgaggccgacctcccgacggaggcttaccgccagcacctccgggcgaagctgcccgagtacatggtgccgtccgcgttcgtcgggctgccctcgttgcccctctcgcccaacggcaaggtggatcgcaaggcgctgcccccgccggagcccgctgccgagggagctctcgagttcgtggccccgcgagggcccgtcgaggcgatgctggccgagatctggagccgcttgctcggggtcggccaggtcggcgcgcaggacgacttcttcgcgctgggcggccactcgctgctcgcgacgcaggtcgtctcgcgcatccgcgcggccttcggggtggagctgcccctgcgcgcgctcttcgaggccccgaccgtggcggggctcgcggcgcgcctcgacgacggcgggcgcgtccaggctgccgctgacccgcgcggcgcgaccggacgtcctgcccctctcgttcgcgcagcggcgcctgtggttcctgcagcggatggatggccccggcgccacctaccacatccccttcgccctgcacttccagggggagctggacctgccggccttgcaggctgcggtcggcgacgtcatggcccggcacgagagcttgcggaccgtgttccccgtcgtcgacgaggtgcctcaccagcgcatcctcgacgtggacgccgcgcccctccggtggaccgtcacgccggcggcccccgccgcgctgcccgggctgctgaccgaggcgacccagcggggcttcgatctggcggtcgagcctccgctgcgcgcggaggtgttctcgctcggccccgacgaccacgtgctcttgctcctgctccaccacatcgccggtgacggctggtcgatggggcccctgcgcgcggatctcaccgccgcgtacctggcgcgccgtcagggcaaggctcctggctggagcgcgcttcccgtgcagtacgccgactacaccctgtggcagcaccggctcctcggcgagcagcgcgatccggacagcctgttcgccacccagctcgcgtactggacccggaccctcgccggcctcccggagcagctcccgctgcccgccgatcgtcctcgcccggcggtggcctctcaccggggtggcgtcgtcccgttccggctgggaccggccttgcacgaggggctcctcgacctcgctgcgcaggggggcgccagcctgttcatggtgctgcaggccggcctggctgcgctcctgtcgcggctcggtgcaggggacgacatcgtggtggggagcccgatcgccggacgcaccgaccacgccctcgaccacctcgtcgggttcttcgtgaacacgctggtgctgcgcaccgacacctcgggagatcccagcttcctccagctcctcggccgggtgcgcgaggccgccctcggggcttacgcccaccaggacgtgccgttcgagtacctggtcgaggtcctgaaccccgtccgctcgctgtcccaccaccccctgttccaggtgatgctggtgctgcagagccaccaggacgacggcatcgacctgcccgggctgcgcgtggctgcgatgccggtctcgctggagaccgccaagttcgatctgctgttcgcgctgagcgagcggcgcggggcggatggtgcccgcgagggcctcgacggcgtgatcgagtacgccagcgatcggttcgaccccgggaccgtcgaggggatcgtggcgcggtggctccgcctgctcgaggctgccgtggccgatcccgggctgccgatccgacggatcgagctgctcacggtggacgagcgtcggacgttgctcgacacgtacaacgacaccgcccgccccgtccccgagaccagcttgcccgcgctgttcgaggcgcaggccaagatggcacctgcgcgcccggccctggtgttcgaggacgccgtgctgacgtacgccgagatcaacgcccgcgccaaccgcctggcgcacgtgctgatcgcgcagggggtcggcccggagcgcatcgtcgccttgctcttgccgcgcacccccgagctgatcgtcgcgctcctggcgacgctcaagacgggggccgcctacctgcccgtggacccggagtaccccgcgtcacggatcgcgacgatgctgagcgacgcccgccctgcggtcgtgctggcgagcctggagactgcgcgcgcgatccccgagggcatcacgttcccctgcctggtggtggacgagcccgacacggctgccgcggtgtcccgtcatcgcgccaccgacccgacggacgtcgagcgcaccgttgccttgatgccgcagcatccggcgtacgtgatctacacgtccggatcgaccggcatccccaagggcgtggtcatgccctccggcgccctggtgaacctgctgttctggcaccagcgcgccttgccgagcggcgagggcacccgcgtcgcgcagttcacggccctgagcttcgacgtctcggcgcaagagatcctctccacgctgctcttcgggaagaccctggtcgtgccgccggacgccgtgcggcgcagcgcggagcggctggcgggctggctcgcgaagcaccgcgtcgaggagctgttcgctccaaacctcgtcgtggaagcgctggccgaggccgccctcgagcgaggcctcaccttgccccatctgcgcgacatcgcgcaggcaggcgaagcgctcaccctgagtcgccacgtgcgcgagttccaccgtcgaacgcccggccgccgcctgcacaaccactacggtccggcggagacacacgtggccaccggctgcacgctgcccgccgatctcgcgacctgcacgctgccgccgtccatcggccagccgatcttcaacacgcgcgtgtacgtgctggatgaccggctggacctgacgcctgccggcatcgcaggggagctgtacctcaccggggccgggctcgcgcgaggctacctggaccggcctggcttgacggcccagcggttcatccccgaccccttcggccccccgggcgcgcgcatgtaccgcaccggagaccaggcgcggtggcgcgcagcgggggagctggagttcctcggccgcctcgaccaccaggtcaagatccggggcttccgcatcgagctgggcgagatcgaggcggtgctggccgcgcatcccgagctttctcgggcggcggtcctcgcccgcgatcaccagtcgggagggaagtggctggtggcctacgtcgtccctgtgccgcacgctgccccgcggcccgaggccttgcgcgagcacctgcgccagcggctccccgattacatggtccccggggccgtggtggtcctggagcgcctccccctgacgctgaacgggaagctcgatcgccaggcgctgcctgcgccggagctgagcccggaacgggcggggaggggagcgcagaccccgcaggagcagctgctgtgcgacctgttcgccgaggtgctggggctggggcaggtgggcatcgatgaggacttcttcgaactgggcggtcactcgctgctggcgacgcggttgatcggccggatccgcgccaccctgggcgtggaggtgccgctccaggcgctgttcgaagccccgacggtggccggcctctcgacgcagctcgacggcgcccaggcggcacgaccggcgctgcgggtgcaggcccgcccggacgcgctgccgctgtcgttcgcgcagcagaggctgtggttcctgcaccagatggagggccgcaccgcgacgtacaacctggcgctggcgctgcgcctgaccggtgccctcgaccgggtggcgctgcaggcggccctcggcgatgtcgtcgcgcgccacgaaagcttgcggacggtgttcccgcacgccgacgggaccccctcccaggtggtgctcgatgccgacgcggcgcgccccgcgctcaccgtcacccggaccgacgcggagagcgtacgtgacgcgctgaacacggcggtgcgtcatggcttcgatctgtccgtcgagccaccgctgcgggccacgctgttcgaggtggcgcccgaggtccacgtgctgctgctgacgatgcaccacatcgtcggtgacggcggctcgatggaacccctttcgcaggacctggccaccgcgtatgccgcgcgctgccagggggaagcgccggcctggtcgccgcttccggtgcagtacgccgactacacgctctggcagcgggagctgctcggcgaccaggccgacgccgagagccggttcgcgcagcagctcgcctactggaccagggaactggcgggcctccccgagcagctcacgctacccaccgaccgcccgcgcccgcgggtggcctcctaccggggaggggtggtccagatggcgtgggacgcctccttgcaccagggcctgatcgccctcgcgcgcaagaacggcgccagcttgttcatggtgctccaggctggcctcgccgccttgttcatgcggctgggagcgggtcacgacatcgcgctgggcagcccgatcgcgggtcgcaccgaccatgcgctcgacgacctggtcgggttcttcgtcaacacgctggtgctgcgcgcggacacgtcggggaacccgagcttccggcagctgctgtgccgcgctcgtggagtggccctggccgcctacgcccatcaggacgtgccgttcgagtgcctggtcgaggcgttgaacccgacgcgatcgctggcacaccacccgctgttccaggtcatgctcggcgtgcagcgcgcccagccgaaggacatcgagctgtctggtctgcacgtcgagccggcagagaccggcaccacggccaccgcgcgcgtcgacctgacgttcagcgtcaccgagcgccgcagcgccgagggcgctgcggagggcatcgagggggtggtcgagtacagcagcgatctgttcgacgccgcctcggtcgagacgctggtggcgcggtgggcgcggctgctggaggccgccgtcgcggatccggagcagcccatcgggaacctggaggtcctgacggctgacgagcgccggaggctgctggtcgaccacaacgcgaccgcccatccggtcgcggccatcagcctgagcgcagcgttccaggcgcaggtggaggcgacgccggacgcggtggcggtggtgtgcgacggcacggcgctgacgtacgccgagctgaacgcgcgggcgaaccggctggcgcaccagctgatcgcgcagggggtagcgctggagagccgtgtggcgctggcgctggagcggtcgctggagctggtgctggccctgctggccgtcatcaaggccgggggagcttacgtgcccctggatgcgcgctacccgcaggcgcggagagcgcacatcctgaaggaaacgggcgcagtggtgctgctggccagcggggaggggagcgacgacaccgcgtcgctgggcgtcccggtgctgctggtcgacgctggttccgtcgcgtccgatccgggcgcgccggttgtcgtctgcgatccggaccagctcgcgtacgtcatgtacacgtcagggtcgacggggcagccgaaggggatcggcgtcacgcaccggaacgtggtggagctggcctcggatccgtgctggcgctcggggcatcaacggcgggtgctgtggcactcaccgccggcgttcgacgcctcgacctacgagttctgggtgccgctcctgggtggcgggcagatcgtcgtttcacccgctggtgagcagaccgcccatgatctccggcgcgtgatctccgagcaccaggtcaccagcgtcttcctgacgacggcgctgttcaacctgatggtggaggaagacccgagcagcttccacacggtgggcgaagtgtggaccggcggcgaagcggtctcgccgcagtcgatgcaacgggtgctggacacctgcccggacacgatgatcgcccacgtctacggcccgacggagacgacgacgttcgccacgttcgaggccctgcgaccgccgcaccacatcgagggcacggtgccgatcggcaagccgatggcgaacatgcgggcttacgtgctcgatgaaggcttgcggcccgtgccagaaggcgtgcccggggagctgtacctcgcgggcgccgggctctcgcgcggatacgtcgcgcgccctggactgacggccgagcgcttcgtcgtcgacccgttcgccagcggcgagcgcatgtaccgcaccggcgatcgtgtccggtggaacgctggcgggagcctcgacttcctgggccgcaccgacaaccaggtgaagatccgaggcttccgcatcgagccggacgagatcggcgcggtgctgctggagcatcccgaggtcgcgcaggcggcggtcgtcgtccgcgaggaccggcctggcgagaagcggctgatcgcttacgccgtcgccaccgcggggacgaaccccgacccgcgggcgctgcgcgactggagcaagcagcggctgccggagttcatggtgcccgccgcgctcgtcctgctcgacgccttgccgctgaacgcgaacggcaagctcgaccgcaaggcgctgccggcccccgatctcggaccgtctcgcgctggcagagcgccacgaacccagcgcgagcacctgctctgcgatctcttcgccgaggtcctcggcctgccgcgcgtcagcatcgacgacgacttcttcgagctgggcggccactcgctgctcgccacccgcctcgtcagccgcgtgcgcaccaccctcggcgtcgagctgagcgtccgcagcctcttcgagagtcccaccgtggccgggctgtgcggccgtctggagagggacgacgccagcaccgtgcgcctggccttgcgcgcccaggcccgtccggaccgccttcccctgtcgttcgcgcagcagcgcctgtggttcttgcaccagatggaaggccgctctgcgacctacaacatccccatggccctgcgtctgacggggacactcgaccgcgcggcgctggaggccgcactgggcgacgtggtcacccgtcacgagagcctccggacgaggttctctcagcacgacggcaccgcctaccaggccatcctggctcccaccgaggcgcgcccgtcgctgtccgtcaccgtgaccacggatgcggagctgccggaggccctggccgcggccgctcagtacggcttcgacctcgcgcacgagctgccgctgcgcgccgagctgttcgtgctgggccctggcgagcacctgctgctgctcctgctgcatcacatcgccggtgatggctggtccctcgcgcccttgtcgcgcgacctcgcgaccgcgtacacggcccggtgcggaggcgaagcgccggcgtggacgccgttgccggtccagtacggcgactacaccctctggcagcacgccttgctgggaggcgtcgccgatcccgacagcctgttcagccgccagctcgcgtactggacccggaccctcgctgatctccccgagcgcatcgagctgcccgccgatcgcccgggcccggcggtcgcctcgtaccggggcgactacctccccgtgcagatcgacgccgccctgcaccgcggcctgcacggcctcgcccgacagagcggcgccagcctgttcatggtgctccaggccggactcgcggcgctcctgtctcgcctcggcgcgggcgacgacatccccctgggcagccccatcgccgggcgcacggatcgcgcgctggaggacctggtcggcttcttcgtcaacaccctggtgctgcgcacggacacctcggggaatcccagcttccgacagctcctcggccgcgtgcgggagacggcgctcagcgcctacgcccaccaggacatgccgttcgagcacctcgtcgagatcctcaaccctgccaggtcgctctcgcaccaccccctgttccaggtgctgctcgcggtccagaacgcgcctgaaggcgccttcacgctgcctggcctggacgtctccttcgtctccacccgcaccggcacctccaagttcgacctcggcttcagcctgtccgaacagcgcggcgcggacggttccccgcaagggctggccggctacgtcgagtacagcaccgaccgcttcgacctcggcaccgtcgagaccctgttctcgcgctggatccgcttgctggaggctgcggtggagcacccggatcgcccgatcggggccaccgagctgctctccgcgcgcgagcgccacaccctcctcgtcgagcgcaacgacaccgcccagcccctccccgaggccacgttcccgaccctcttccaggcacaggtcgaggcgacgcccggggcagtggcgctggcatgggacgaggcccagctcacctacggcgagctgaacgcccgggccaaccagcttgcgcacaggctgcgcgcggaaggcgtgggacccgagcacctcgtggccctggccatgccccgctcacccgacctggtgatcgcccttctggccgtgctgaaggccggcgcggcctacctcccggtggacccggactaccccgccgcgcggatcgccttcatgctcaccgacgcccggcccatcctgctgctgacccgcctcgacacgcccgcggccgcgttcgagagcatccccacgcccaggctggtggtcgacgaccccgccacgatccgcgcgctcgccgatctccccgccagcaacccggtggtggccgtgctgccgcagcaccccgcgtacgtcatctacacctcgggctcgaccggagttcccaagggcgtggtcgtgagccaccagggcatcgccagcctggcgaaggcccacatcgagcggttcggtgtgaccgcgcagagccgcgtgctccagttcgcctcgcccagcttcgatgcctcgttcgcggacctggccatgaccttcctttcgggcgcggcgctggtgctggcaccgaaggaacagctgcagccgggcgctccgctggccgcgctgacgagccgacagcgggtgacgcacgcgacgctccccccggccgccctctcgatcatgtcaccgcagggcggcctccccgctgacatgaccctggtcgtggccggcgaggcctgcccgcccgagctggtcgcagcctgggcacccgggcgacggatgatcaacgcctacggccccaccgagaccacggtctgcgccacactgagcgagctgttgccgcccgccgcagccatcccacccatcgggagacccatcgtgaacaccagggtctacgtgctcgatgcgggcctccagcccgtgcctcccggcgtggccggggagctctacgtcgccggcgcgggtctggcacggggctacctgggcaggccaggcttgacggcggcgcgcttcgtcgcgagccccttcggcgacggcgcgcgcatgtaccgcaccggcgaccgggcgcgctggaacgcggacgggagcctcgagttttgcggacgagccgacgatcaggtcaagcttcgcggcttccggatcgagctcggcgagatcgaagcccagctctccgcgcaccccgaggtcgcgcaggccgccgtggtggtccgccaggatggccaggctgccgacaggcgcctggtcgcctacgtcgtcgccgcagagcgggacggcaaggaccgcaacgagcagatcgagcacgaccaggtgcgcgcgtggcagcagatctacgagacccactacgcgaccgtggacgcgacccggttcgggcaggacttcagcggctggaacagcagctacgacggagagcccatcccggtcgagcagatgcgcgagtggcgcgacgccaccgtcacccgcatcctctcgctgcgcccgaggcgcgtcctggagatcggggtcggcaacgcgctgctcctctcgcagatcgcgccccactgcgagagctactggggcaccgacctctcggccacggtcatcgcctcgctggcgacgcagctcgagcacctgcccgagctgtcggagaaggtcgtgctgcgcgcccagcccgcccacgacctcggcgggctgcccgcgggaacgttcgacacgatcgtcatcaactcggtcgtgcagtacttccccaacaccgactacctcgtcgacgtgctgaaccaggcgctccagctcctcgtccctggtggggcgctgttcgtcggcgatgtgcgcaacgtgcagctcctgcgctgcttcgccaccgccgtccagcttcgccgcgccgaggacggcgcggaggaggccgcgctgcgccacgcgatcgagcacgccctgcgggtggagaaggagctgctcgtcgcgcccgagttcttcgcggccctcgcggcgtcgcatccggacatcggtggcgtggacgtccgcctcaagcgcggccagcaccacaacgagctgacccgctaccgctacgacgccatcctgcgaaaatcacccatcccagcgctctcgctggccgaggcccccacgctgcgatgggaagcgtgcggcggcatcccagccctcgaagcgctgctcgcgggcgagcgccccgaccggctacgcctgagtggcgtcccgaaccgccgcatccaccaggaagccgccgccctgcgcgtcttcgaggaaggccatcccgtgagcgcatcgcggaagctcctggaggacagcctcccggaggcgctcgatccagagtccctcgtcgcgctgggagaacgtcacggctactgggtggccgtcacctggtcgccgacctcggtcgacgccgtcgacgtcctgttcgtgcaggccgagacggtagcctcggctgcacccgtcgacgtccacacgccctccggcatcgcgggcatgccgctgtccgcgttcacgaacaacccctcgaccgcgcgagggaccggggcactgatcgccaccctccgggagcacctccgcgagcggctccccgactacatggtgcccgcagccgtggtcgtcctggagcgctttccgctctcccccagcggcaagctcgaccgccaggcgctgcctgcgccggagctgggtcaggaccgcgcgggacgagcggcgcgcacgccccaggaacagatgctgtgcgacctgttcgccgaggtgctggggctgggggaggtgggcatcgacgaggacttcttcgcgctgggcggtcactcgctgctggcgacgcgattgatcggccggatccgcgccaccctgggtgtggaggtgccgctccgagcgctgttcgaagcgccgacggtggcccgtctggccacccagctcggcgacgccggagcggcgcggccggcgctgcgggtgcaggcccgcccggacgcgctgccgctgtcgttcgcgcagcagaggctgtggttcctgcaccagatggagggccgcaccgcgacgtacaacatgccgctggcgctgcgcctgaccggtgcgctcgaccggacggccctccagacggccctgggtgacgtgatcacgcgccacgagagcctgcggacggtgttcccgcaggtggaagggatgcctttccaggtggtcctcgacgccgacaaggcgcgtcctgtgttgaccctcctccggaccgacgagaagggcctgcgcgaggcgctggccaccgcagcccgacacggcttcgacctgtccgtcgagccaccgctgcgggccacgctgttcgaggtggcgcccgaggtccacgtgctgctgctgacgatgcaccacatcgtcggcgacggctggtccatggggcccctctcgcgcgacctcgccgctgcctatgccgcgcgctgccagggggaagcgccggcctggtcgccgcttccggtgcagtatgccgactacacgctctggcaacgggagctgctcggcgaccaggccgacgccgagagccggttcgcgcagcagctcgcctactggaccagaaccctcgccgacctccccgagcagctggagctgcccaccgatcgcccacgcccgccggtggcctcctaccagggcagcgtgctcccggtgacctgggacgcgcacctgcatcagggcctcgccgatctcgcccgccagagcggcgccagcttgttcatggtgctccaggccggcctcgccgccttgttcacgcgcctgggcgcaggccatgacgtcgccctgggcagccccatcgcgggtcgcaccgatcccgcgctcgacgacctggtcgggttcttcgtcaacacgctggtgctgcgcacggacacgtcggggaacccgagcttccggcagctcctgggccgcgttcgtgaaacggccctggccgcctatgcccatcaggacgtgccgttcgagttcctggtcgaggcgctgaacccggcgcggtcgatggcccatcaccccctgttccaggtcatgctcggcgtccagaacgcgcccgcgggcgccttccagcttcccggactgcacgtggaaccgatgggcacgggcggtacggagacctcacgcgtcgacctgacgttcagcgtcaccgagcgccgcaccgccgagggcgccgcggaaggcatcgagggggtggtcgagtacagcagcgacctgttcgacgccgccacggtcgaggcgctggtggcacggtgggcgcggctgctggaggccgccgtcgcggacccggatcagcccatcgggagcctggagatcctgacggccgaagagcgccagaagctgctggtcgaccacaacgccacggcccatccggtcgcggccatcagcctgagcgcagcgttccaggcgcaggtggaggcaacgccggacgcggtggcggtggtgtgcgacggcacggcgctgacgtacgccgagctgaacgcgcgggcgaaccgactggcgcaccggctgacggcgcatggggtgtcaccggagagccgtgtggcgctggtgctggagcgctcgctggagctggtggtgggcttgctgggggtgatcaaggccggtggcgcgtacgtgccgctggacgcgcgctacccgcaggcgcggagagcgcacatcctgaaggaaacgggcgcggtcgtgctgctggccagcggggaggggagcgaggacaccgcgtcgctgggcatcccggtgctggtggtcgatgctggacccgtggtctccgatccgggctccccggccgcggactccgatccggaccagctcgcgtacgtcatgtacacgtcggggtcgacggggcagccgaaggggatcggtgtcacgcaccggaacgtggtggagctggcctcggatccatgctggcgctcggggcatcatcgtcgggtgctgtggcattcccctccggcgttcgacgcctcgacgtacgagttctgggtgcctctgctgggtggcgggcagatcgtcgtcgctcccgccggggagcagaccgcccacgacctgaggcgtgtgctccgtgaacatcgggtcaccagcgtcttcctgacgacggcgctgttcaacctgatggtggaggaagacccgagcagcttccgcacggtgggcgaagtgtggaccggcggcgaggccgtctcgcctcaggcgatgcagcgggtgctggatgcctgtccggacacgatgatcgcccacgtctacggcccgacggagacgacgacgttcgccacgttcgaggccctgcgaccgccgcaccacatcgagggcacggtgccgatcggcaagccgatggcgaacatgcgggcctacgtgctcgacgaaggattgcggcccgtgccagaaggcgtgcccggggagctgtacctcgcgggcgccgggctctcgcgcggatacgtcgcgcgctccgggctgacggccgagcgcttcgtcgtcgacccgttcgccagcggcgagcgcatgtaccgcaccggcgatcgtgtccggtggaacgccgacgggagcctcgacttcctgggccgcaccgacaaccaggtgaagatccgaggcttccgcatcgagccggacgagatcggcacggtgctgctggagcatcccgaggtcgcgcaggcggcggtcgtcgtgcgcgaggaccggcctggcgagaagcagctgatcgcttacgccgtcgccaccgcggaaacttctcccgacccgcgtgcgctgcgcgactggctcaagcaccgcctgcccgagtacatggtgcccgccgcgctcgtcctgctcgacgccttgccgctgaacgcgaacggcaagctcgaccgcaaggcgctccccgcacccgacctcggtcccacccgcgtcggccggtcaccgcgcaccccgcgcgagcacctgctctgcgacctcttcgccgagatcctcggcctgccacgcgtcggcatcgacgacgacttcttcgagctgggcggccactcgctgctcgccacccgcctcgtcagccgcgtgcgctccaccctcggcgtcgacatgggtctgcgccgcctgttcgaggcgcccaccgtcgctgggctcgcagcctgcctcgatctcgacaccaccgacgacgccttcgaggttgtcctccccctgcgcgcttccggacgcttgcccccgctcttctgcatgcacccgggtggtggcatgagctggagctacgccggcctgatgcgccacctcgacccggagacgcccctctacggcatccaggcgcgcagcctcgctcgacccgagccgcgcccgacctccctccaggccatggccagcgactacgccgaccagctccagcggatccagcctctgggaccctaccacctcctcggctggtcctccggcggcctcgtcgctcacgccgtcgccaccgagctgcaacggcgtggcgccgaggtggcgctgctcgccctcctcgacgcctatcccctggtcgacatcgccctcgacgagcccctggtgcagagcgaacgcgccatcctcgccgggatgatcgaagccgacccgagcgacctgcagggcatggatgaccagcaagcggtcacgcacgtcctcgaagtcctccgccaccagggcaacgtgctggccagcctcgacgcgcgccagatccgcaccctcatcgacctcatgacccacaacgccggcctcgtctccgacttcgtccctgccgtgtaccagggcgacctggtgctcttcagcgccaccatcaaccgcccagatccggcgcgaccggcgctctggcagccctacgtcagcggcgccatcgagaaccatgacatcgagatccgtcacgaccacatgatgcagcccgcgccgctcgcccagatcgggcgcatcgtcgcggccaggctacagaccctccaccgctcacccgaaacgtctccccggaagatcgaacca

SEQ ID NO: 29 - protein, 5076 aa, Chondromyces crocatus
MRPRTGGWSRTWSPPRPTSRRRLTASTSGRSCPSTWCRPRSSGCPRCPSRPTARWIARRCPRRSPLPRELSSSWPREGPSRRCWPRSGAACSGSARSARRTTSSRWAATRCSRRRSSRASARPSGWSCPCARSSRPRPWRGSRRASTTAGASRLPLTRAARPDVLPLSFAQRRLWFLQRMDGPGATYHIPFALHFQGELDLPALQAAVGDVMARHESLRTVFPVVDEVPHQRILDVDAAPLRWTVTPAAPAALPGLLTEATQRGFDLAVEPPLRAEVFSLGPDDHVLLLLLHHIAGDGWSMGPLRADLTAAYLARRQGKAPGWSALPVQYADYTLWQHRLLGEQRDPDSLFATQLAYWTRTLAGLPEQLPLPADRPRPAVASHRGGVVPFRLGPALHEGLLDLAAQGGASLFMVLQAGLAALLSRLGAGDDIVVGSPIAGRTDHALDHLVGFFVNTLVLRTDTSGDPSFLQLLGRVREAALGAYAHQDVPFEYLVEVLNPVRSLSHHPLFQVMLVLQSHQDDGIDLPGLRVAAMPVSLETAKFDLLFALSERRGADGAREGLDGVIEYASDRFDPGTVEGIVARWLRLLEAAVADPGLPIRRIELLTVDERRTLLDTYNDTARPVPETSLPALFEAQAKMAPARPALVFEDAVLTYAEINARANRLAHVLIAQGVGPERIVALLLPRTPELIVALLATLKTGAAYLPVDPEYPASRIATMLSDARPAVVLASLETARAIPEGITFPCLVVDEPDTAAAVSRHRATDPTDVERTVALMPQHPAYVIYTSGSTGIPKGVVMPSGALVNLLFWHQRALPSGEGTRVAQFTALSFDVSAQEILSTLLFGKTLVVPPDAVRRSAERLAGWLAKHRVEELFAPNLVVEALAEAALERGLTLPHLRDIAQAGEALTLSRHVREFHRRTPGRRLHNHYGPAETHVATGCTLPADLATCTLPPSIGQPIFNTRVYVLDDRLDLTPAGIAGELYLTGAGLARGYLDRPGLTAQRFIPDPFGPPGARMYRTGDQARWRAAGELEFLGRLDHQVKIRGFRIELGEIEAVLAAHPELSRAAVLARDHQSGGKWLVAYVVPVPHAAPRPEALREHLRQRLPDYMVPGAVVVLERLPLTLNGKLDRQALPAPELSPERAGRGAQTPQEQLLCDLFAEVLGLGQVGIDEDFFELGGHSLLATRLIGRIRATLGVEVPLQALFEAPTVAGLSTQLDGAQAARPALRVQARPDALPLSFAQQRLWFLHQMEGRTATYNLALALRLTGALDRVALQAALGDVVARHESLRTVFPHADGTPSQVVLDADAARPALTVTRTDAESVRDALNTAVRHGFDLSVEPPLRATLFEVAPEVHVLLLTMHHIVGDGGSMEPLSQDLATAYAARCQGEAPAWSPLPVQYADYTLWQRELLGDQADAESRFAQQLAYWTRELAGLPEQLTLPTDRPRPRVASYRGGVVQMAWDASLHQGLIALARKNGASLFMVLQAGLAALFMRLGAGHDIALGSPIAGRTDHALDDLVGFFVNTLVLRADTSGNPSFRQLLCRARGVALAAYAHQDVPFECLVEALNPTRSLAHHPLFQVMLGVQRAQPKDIELSGLHVEPAETGTTATARVDLTFSVTERRSAEGAAEGIEGVVEYSSDLFDAASVETLVARWARLLEAAVADPEQPIGNLEVLTADERRRLLVDHNATAHPVAAISLSAAFQAQVEATPDAVAVVCDGTALTYAELNARANRLAHQLIAQGVALESRVALALERSLELVLALLAVIKAGGAYVPLDARYPQARRAHILKETGAVVLLASGEGSDDTASLGVPVLLVDAGSVASDPGAPVVVCDPDQLAYVMYTSGSTGQPKGIGVTHRNVVELASDPCWRSGHQRRVLWHSPPAFDASTYEFWVPLLGGGQIVVSPAGEQTAHDLRRVISEHQVTSVFLTTALFNLMVEEDPSSFHTVGEVWTGGEAVSPQSMQRVLDTCPDTMIAHVYGPTETTTFATFEALRPPHHIEGTVPIGKPMANMRAYVLDEGLRPVPEGVPGELYLAGAGLSRGYVARPGLTAERFVVDPFASGERMYRTGDRVRWNAGGSLDFLGRTDNQVKIRGFRIEPDEIGAVLLEHPEVAQAAVVVREDRPGEKRLIAYAVATAGTNPDPRALRDWSKQRLPEFMVPAALVLLDALPLNANGKLDRKALPAPDLGPSRAGRAPRTQREHLLCDLFAEVLGLPRVSIDDDFFELGGHSLLATRLVSRVRTTLGVELSVRSLFESPTVAGLCGRLERDDASTVRLALRAQARPDRLPLSFAQQRLWFLHQMEGRSATYNIPMALRLTGTLDRAALEAALGDVVTRHESLRTRFSQHDGTAYQAILAPTEARPSLSVTVTTDAELPEALAAAAQYGFDLAHELPLRAELFVLGPGEHLLLLLLHHIAGDGWSLAPLSRDLATAYTARCGGEAPAWTPLPVQYGDYTLWQHALLGGVADPDSLFSRQLAYWTRTLADLPERIELPADRPGPAVASYRGDYLPVQIDAALHRGLHGLARQSGASLFMVLQAGLAALLSRLGAGDDIPLGSPIAGRTDRALEDLVGFFVNTLVLRTDTSGNPSFRQLLGRVRETALSAYAHQDMPFEHLVEILNPARSLSHHPLFQVLLAVQNAPEGAFTLPGLDVSFVSTRTGTSKFDLGFSLSEQRGADGSPQGLAGYVEYSTDRFDLGTVETLFSRWIRLLEAAVEHPDRPIGATELLSARERHTLLVERNDTAQPLPEATFPTLFQAQVEATPGAVALAWDEAQLTYGELNARANQLAHRLRAEGVGPEHLVALAMPRSPDLVIALLAVLKAGAAYLPVDPDYPAARIAFMLTDARPILLLTRLDTPAAAFESIPTPRLVVDDPATIRALADLPASNPVVAVLPQHPAYVIYTSGSTGVPKGVVVSHQGIASLAKAHIERFGVTAQSRVLQFASPSFDASFADLAMTFLSGAALVLAPKEQLQPGAPLAALTSRQRVTHATLPPAALSIMSPQGGLPADMTLVVAGEACPPELVAAWAPGRRMINAYGPTETTVCATLSELLPPAAAIPPIGRPIVNTRVYVLDAGLQPVPPGVAGELYVAGAGLARGYLGRPGLTAARFVASPFGDGARMYRTGDRARWNADGSLEFCGRADDQVKLRGFRIELGEIEAQLSAHPEVAQAAVVVRQDGQAADRRLVAYVVAAERDGKDRNEQIEHDQVRAWQQIYETHYATVDATRFGQDFSGWNSSYDGEPIPVEQMREWRDATVTRILSLRPRRVLEIGVGNALLLSQIAPHCESYWGTDLSATVIASLATQLEHLPELSEKVVLRAQPAHDLGGLPAGTFDTIVINSVVQYFPNTDYLVDVLNQALQLLVPGGALFVGDVRNVQLLRCFATAVQLRRAEDGAEEAALRHAIEHALRVEKELLVAPEFFAALAASHPDIGGVDVRLKRGQHHNELTRYRYDAILRKSPIPALSLAEAPTLRWEACGGIPALEALLAGERPDRLRLSGVPNRRIHQEAAALRVFEEGHPVSASRKLLEDSLPEALDPESLVALGERHGYWVAVTWSPTSVDAVDVLFVQAETVASAAPVDVHTPSGIAGMPLSAFTNNPSTARGTGALIATLREHLRERLPDYMVPAAVVVLERFPLSPSGKLDRQALPAPELGQDRAGRAARTPQEQMLCDLFAEVLGLGEVGIDEDFFALGGHSLLATRLIGRIRATLGVEVPLRALFEAPTVARLATQLGDAGAARPALRVQARPDALPLSFAQQRLWFLHQMEGRTATYNMPLALRLTGALDRTALQTALGDVITRHESLRTVFPQVEGMPFQVVLDADKARPVLTLLRTDEKGLREALATAARHGFDLSVEPPLRATLFEVAPEVHVLLLTMHHIVGDGWSMGPLSRDLAAAYAARCQGEAPAWSPLPVQYADYTLWQRELLGDQADAESRFAQQLAYWTRTLADLPEQLELPTDRPRPPVASYQGSVLPVTWDAHLHQGLADLARQSGASLFMVLQAGLAALFTRLGAGHDVALGSPIAGRTDPALDDLVGFFVNTLVLRTDTSGNPSFRQLLGRVRETALAAYAHQDVPFEFLVEALNPARSMAHHPLFQVMLGVQNAPAGAFQLPGLHVEPMGTGGTETSRVDLTFSVTERRTAEGAAEGIEGVVEYSSDLFDAATVEALVARWARLLEAAVADPDQPIGSLEILTAEERQKLLVDHNATAHPVAAISLSAAFQAQVEATPDAVAVVCDGTALTYAELNARANRLAHRLTAHGVSPESRVALVLERSLELVVGLLGVIKAGGAYVPLDARYPQARRAHILKETGAVVLLASGEGSEDTASLGIPVLVVDAGPVVSDPGSPAADSDPDQLAYVMYTSGSTGQPKGIGVTHRNVVELASDPCWRSGHHRRVLWHSPPAFDASTYEFWVPLLGGGQIVVAPAGEQTAHDLRRVLREHRVTSVFLTTALFNLMVEEDPSSFRTVGEVWTGGEAVSPQAMQRVLDACPDTMIAHVYGPTETTTFATFEALRPPHHIEGTVPIGKPMANMRAYVLDEGLRPVPEGVPGELYLAGAGLSRGYVARSGLTAERFVVDPFASGERMYRTGDRVRWNADGSLDFLGRTDNQVKIRGFRIEPDEIGTVLLEHPEVAQAAVVVREDRPGEKQLIAYAVATAETSPDPRALRDWLKHRLPEYMVPAALVLLDALPLNANGKLDRKALPAPDLGPTRVGRSPRTPREHLLCDLFAEILGLPRVGIDDDFFELGGHSLLATRLVSRVRSTLGVDMGLRRLFEAPTVAGLAACLDLDTTDDAFEVVLPLRASGRLPPLFCMHPGGGMSWSYAGLMRHLDPETPLYGIQARSLARPEPRPTSLQAMASDYADQLQRIQPLGPYHLLGWSSGGLVAHAVATELQRRGAEVALLALLDAYPLVDIALDEPLVQSERAILAGMIEADPSDLQGMDDQQAVTHVLEVLRHQGNVLASLDARQIRTLIDLMTHNAGLVSDFVPAVYQGDLVLFSATINRPDPARPALWQPYVSGAIENHDIEIRHDHMMQPAPLAQIGRIVAARLQTLHRSPETSPRKIEP

SEQ ID NO: 30 - DNA, 1317 bp, Chondromyces crocatus
gagggcagggtgcctgcgtcgtcggggcagcggcggctctggttggtcgagcggctcgccgccgagcgcacgctgtacaacgtgcacctctgcgtgcgcatggaggggccgctcgatccgtcatggctccgacagagcgtggccatgctcttcgagcgacacgaggtgcttcgcatgcggctccacgaggtcgacggggatgtcctcgggatcgtcagccccccgggtgaggtggagctgcccctcgtcgaccttcgccaggtgccacccgaagcccgagggcagcggttctctcaggtctcgatggatcacagcctcacgcccctggatctcggtgtcgggcctgtcgtgcggatgacgctggtggcgctgaaggacgacgagcacgtcctcctggtcacgcagcaccacgccgtcaccgacgggcggtcgctcatgctcctgccggcggagctcttcgccttctaccgcgcgctctgcgatggaacgtcgcctcgtctgcccaccctgcccatcacctacgcggacttcgtggtctgggaggcccaggcgcggcagtcgccgcacttcgccgcgcatctggcgtggtggcagacccgcctctcgaacctccccgagctggagcttcccttcggtcgcaaggtcgaagcgcccacgtacaccggggacttcgtgacgttcgtgtacccgctcgtgctcacctccgggctggagtcgatcgcggcgcggcacgggagcaccctgttcagggtcctggtggcggcctgggctgccttgctccaccgctacaccggtcagaccgacttccccatcggcacggtcacggccatgcgcagggacccccagctgcatggcctcctcgggtacttcgcccacaccctcgtcttgcgctgcgagctggaggccgaccagacgttcctcgatctcgtggcccggatcgacggcgtggtgcgggaagcgctggcgcacgcagaggtgcctttcgacgacatcgtccgtgccgtgggggcctcgcgtcgaggacacctcaacccgctggtccagtcctccttcgtgctcgagaactactcgttccacgctcacgaagccgccgatcagcggtggacgccgtacttcgaggagatcgacgcgggcgtgaagggaggggcgaaattcgacgtctccatggccctctacgtgacgcccgagggcttgaaggggaagctcgagttcgcgacggatctgttcgagcgcgccgccatggaacggctggtgagccacttcgaggcgttgctcctcgatgtggtcacccacccggcccggcgtttgtcggatctg

SEQ ID NO: 31 - protein, 439 aa, Chondromyces crocatus
EGRVPASSGQRRLWLVERLAAERTLYNVHLCVRMEGPLDPSWLRQSVAMLFERHEVLRMRLHEVDGDVLGIVSPPGEVELPLVDLRQVPPEARGQRFSQVSMDHSLTPLDLGVGPVVRMTLVALKDDEHVLLVTQHHAVTDGRSLMLLPAELFAFYRALCDGTSPRLPTLPITYADFVVWEAQARQSPHFAAHLAWWQTRLSNLPELELPFGRKVEAPTYTGDFVTFVYPLVLTSGLESIAARHGSTLFRVLVAAWAALLHRYTGQTDFPIGTVTAMRRDPQLHGLLGYFAHTLVLRCELEADQTFLDLVARIDGVVREALAHAEVPFDDIVRAVGASRRGHLNPLVQSSFVLENYSFHAHEAADQRWTPYFEEIDAGVKGGAKFDVSMALYVTPEGLKGKLEFATDLFERAAMERLVSHFEALLLDVVTHPARRLSDL

SEQ ID NO: 32 - DNA, 1554 bp, Chondromyces crocatus
gtggagcgacgccagctgctggtcgactggaacgagaccgcgagggacttccgtcgagcgacgtgcatccacgagctgttcatggaacaggcctcgcggacaccggaagccgtcgcggtgcacttcgaggaggagcagctcacgtacggcgagctggacgcccgctccaaccagctcgcgcaccacctgcgcgcgctgggcgtggggcccgaggtgctggtgggcctgtgcgtggagcggtccctcgacatggtcgtggggcttctgggcatcgcgaaagccggcggcgctcacgtgccgctggatccggcgtatccgccggagcggctggcgttcatgctggaggacgcgcgcgcgagcgtcctgctcacgcaagcgccgctggtcgagcggctcccggcgatctcggcgcgggtcgtgtgcttcgacgcggatgctcctgcgctggctgcatggccacgctcgaccccggaggtcgtcgtcacgtcggacaacctggcctacgtcatctacacgtcgggctcgacgggcacaccgaagggcgtgatgtgcacgcaccgcgggctcgtcaacctcgtggaccacgaggccgagctcctcgagattggtcaggggaccccggtcctgcagttcgcttcgatctcgttcgacccctccctctcacagctcctcggggccctgagccggggcggaatcgtggttctcgcgtcggccgatcaacggcgctccagcgccgcgctgacagggctgctgcgggcccggggcgtggaggtcgcccacctgccgccgagcgcgctttcgctcctcgacgagagcgatcccctggcgctccgtgtgctgatggtgggcggtgaggtctgccccgtcggtgctgccacggtctgggcccgtgggcgccgtttcatcaactcctacggtccgacggagacgacgatcacggtgtcgtactgggaagggaagccgtcgcccggcgcctccgttccgctcggcaagccgaacgccaacacgcaggtttacgtgctctctcctgcgatgcaggtgctcccgatcggggtgccgggggagctcttcatcgccggcgctggcgtctcgcgtggctacctgaagcgaccgggcctcaccgccgcacgcttcctccctgatcctttcgggccagccgggggcaggatgtaccgcaccggcgacctttgccgctggcgggaggatggcaacctcgagttcctgggccgtatcgaccaccaggtgaagatccggggcttccggatcgagctgggagagatcgagtcggtgctggagcagcaccccgcggtgcgcgcttgcgtggtcatggcgcgcgaggacgagcccggcaaccagcgcctggtcgcgtacgtggtgcctgcggcggacgaggagggctcgatcgctgatctgcgtgcgcacctcaaggcgaagctgccggaccacatgatcccgtcagcgttcgtcgccttgcccgtcctcccgctcagcgcgaacggcaaggtggatcgcaaggccctcccggcccccgacggtcgcgccgaggatcaccgc

SEQ ID NO: 33 - protein, 518 aa, Chondromyces crocatus
VERRQLLVDWNETARDFRRATCIHELFMEQASRTPEAVAVHFEEEQLTYGELDARSNQLAHHLRALGVGPEVLVGLCVERSLDMVVGLLGIAKAGGAHVPLDPAYPPERLAFMLEDARASVLLTQAPLVERLPAISARVVCFDADAPALAAWPRSTPEVVVTSDNLAYVIYTSGSTGTPKGVMCTHRGLVNLVDHEAELLEIGQGTPVLQFASISFDPSLSQLLGALSRGGIVVLASADQRRSSAALTGLLRARGVEVAHLPPSALSLLDESDPLALRVLMVGGEVCPVGAATVWARGRRFINSYGPTETTITVSYWEGKPSPGASVPLGKPNANTQVYVLSPAMQVLPIGVPGELFIAGAGVSRGYLKRPGLTAARFLPDPFGPAGGRMYRTGDLCRWREDGNLEFLGRIDHQVKIRGFRIELGEIESVLEQHPAVRACVVMAREDEPGNQRLVAYVVPAADEEGSIADLRAHLKAKLPDHMIPSAFVALPVLPLSANGKVDRKALPAPDGRAEDHR

SEQ ID NO: 34 - DNA, 195 bp, Chondromyces crocatus
gagctgctcgccgagatctggagcggcctgctcggcgtcgggcggatcggggggcaggacgatttcttcgagctggggggacactcgctcctggcgacgcaattgatcgcgcgcctccgcgccgccttcggcgtcgagctgcccatgcgcggcgtgttcgaggcgcggacgctggcgaagctcgccacggagatc

SEQ ID NO: 35 - protein, 65 aa, Chondromyces crocatus
ELLAEIWSGLLGVGRIGGQDDFFELGGHSLLATQLIARLRAAFGVELPMRGVFEARTLAKLATEI

SEQ ID NO: 36 - DNA, 1284 bp, Chondromyces crocatus
gagcgcgcggtccccctgtcgttcgcccaggagcggctgtggttcctggaccggctcgaacccgacagtcctttttacaacatcccggtggtggtgcgtctcgcggggaacctggacgtgcacgccctcgagcggagcctcggcgagatcgtgcgccggcacgaggcgctgcggacgatctttccggcggacgatgggcaggcccgccaggtggtgacgacgccctccgactggcgcttgcccctcgtcgatgtgcctgcgggcgagctgcgtcggcgcatcgaagcggaagctcgggctccgttccgcctcgcggagggaccgctgttccggggcacgctgctgcggctgtcggagcgagagcacgtgctgctcttgacgatgcaccacatcgtcagcgacggctggtcgatgggggtgctcgtgcgtgagctgggcgcgctctacgaagccttctcggcggggaagccctcgtcgttgcctgcgctgcccgtccagtacccggacttcgcgctgtggcagcggcgcgtgctgagcgaggcgcgcctcgatgcgctgctcgcgtactggcaggcgcagctgtcgggcgcgccgccgctcaccttgccgacggacaggcctcggccgcccgtggcatcccatcgggggagcaccgtcacgttccagcttcctcgtgcgatcggcgaggggctgcgcgcgctgggccgcaaggaaggcgcgacgctgttcatgacgctcctgtcggccttcgcggtgatcctcggccggcacgcgaaccagctcgatttctgcgtggggacgcccgtggcggggcggacgcggagagaggtcgaggggatgctcgggtgcttcatcaacaccctggtcctgcgcgccgacctgtccggggatcccagcttccggagactcatgggccgcatccgcgaggtggcgctcgccgcgtatgcccatcaggacgcccccttcgagcggctggtggagcggctgggcgtttcgcggagcctcgggcacagcccggtgttccaggtgatgttcgtcctccagagcgccccggtggacacgtttcgtcttccgggcctggtgatctcgaccgcgcaggagacgacgagcaccgcgaagttcgatctgaccctctccatggaggagggccccgaggggctctccggcgtgttcgagtacgcgacggacctgttcgatgcggcgacggtcgagcggctggccgggcacttcggcgtgctcctgcgcgcggtcgtgcaagacccggacgcgtcgatcgcgacgctg

SEQ ID NO: 37 - protein, 428 aa, Chondromyces crocatus
ERAVPLSFAQERLWFLDRLEPDSPFYNIPVVVRLAGNLDVHALERSLGEIVRRHEALRTIFPADDGQARQVVTTPSDWRLPLVDVPAGELRRRIEAEARAPFRLAEGPLFRGTLLRLSEREHVLLLTMHHIVSDGWSMGVLVRELGALYEAFSAGKPSSLPALPVQYPDFALWQRRVLSEARLDALLAYWQAQLSGAPPLTLPTDRPRPPVASHRGSTVTFQLPRAIGEGLRALGRKEGATLFMTLLSAFAVILGRHANQLDFCVGTPVAGRTRREVEGMLGCFINTLVLRADLSGDPSFRRLMGRIREVALAAYAHQDAPFERLVERLGVSRSLGHSPVFQVMFVLQSAPVDTFRLPGLVISTAQETTSTAKFDLTLSMEEGPEGLSGVFEYATDLFDAATVERLAGHFGVLLRAVVQDPDASIATL

SEQ ID NO: 38 - DNA, 1605 bp, Chondromyces crocatus
gacgagcggcagcgcgtgctggtgacatggaacgaggggggaacggagccctctcccgtcggctgcctccacacgctgttcatggagcaggcgtcgaggacgccggacgccatcgcggtgcgctgcggtggggagcagctcacgtacgccgagctggatgcccgatccagccgcctcgcacatcacctccggggcctgggcgtgcgcgccgacggcctcgtcgggttgtgtgtcgagcggtccctcgacatggtcgtgggcctcctcgggatcctgaagaccggcggcgcctacgtgccgctggatcctgcgtatccgcaggaccgcctggcgttcatggtgcgggacacgcaggtgcaggtggtggtcacccagtcgcgggtggcgcacgtgctgcccgagagcgaggcgcggctcgtgcggctggacgccgactgggcggagatcgcgcaggcgtccgcggagccgcccgcctccggcgcgacgcctggcaccctggcctatgtcatctacacgtccggctcgacggggacacccaagggcgcgatggtcgagcacggccatgtcgtccggctgttcacggcgacggccgcgtggttccagttcggcgcgcgggacgtgtggacgatgttccactcggtggccttcgacttctccgtctgggagctgtggggtgcgctgctccacggaggccgtgtggtggtcgtgcctcacgcggtgagccgggatcccgaggcgttccacgcgctcgtcgtgcgcgagaaggtgacgatcctcaaccagaccccgtcggcgttccgcgagttcgtccgggtggacgggagcgtctctcatgagacccgtgcggcgctcgcgctgcgccacgtgatcttcggcggggaggcgctcgatgtgggggagctgcggccctggtgggatcggcacgaggacgacgcgcccgtgctggtcaacatgtacgggatcaccgagacgaccgtgcatgtcacccatcggcccctgagccgggcggatctggagcgaccctggtcgagcaccatcgggcgtccgatccccgacctgcaggtgtacgtgctcgatgcggcgcgcaacccggtgcccatcggggtgtccggcgagatgtacgtcggaggagcgggggtctcgcgtggctatctcgggcgcagcgcgctcaccgccgagcgcttcgtcgaggatccattctccgcccggcccggggcgcgtctgtaccggaccggggatctcgcccgctggaacagcgcgggggagctcgagtacctgggccggatcgatcagcaggtgaagatccgggggttccgcatcgagctgggggagatcgaggcggtgctcggggagcaccctgcggtgcgcgcgtgcgtggtcgtggcgcgcgaggacgtccccgggaacaagcgcctggtggcctacgtggtgcccgacgagggcggcgtcccgacggcggcgtaccgtgagcacctgcgggcgaagctgcccgagtacatgatcccggcggccttcgtcgtcctcgacgcgctgccctcgaccccgagcggcaaggtggaccgcagggcgctgcctgcgcccgagcagcgcccggaggacggctgc

SEQ ID NO: 39 - protein, 535 aa, Chondromyces crocatus
DERQRVLVTWNEGGTEPSPVGCLHTLFMEQASRTPDAIAVRCGGEQLTYAELDARSSRLAHHLRGLGVRADGLVGLCVERSLDMVVGLLGILKTGGAYVPLDPAYPQDRLAFMVRDTQVQVVVTQSRVAHVLPESEARLVRLDADWAEIAQASAEPPASGATPGTLAYVIYTSGSTGTPKGAMVEHGHVVRLFTATAAWFQFGARDVWTMFHSVAFDFSVWELWGALLHGGRVVVVPHAVSRDPEAFHALVVREKVTILNQTPSAFREFVRVDGSVSHETRAALALRHVIFGGEALDVGELRPWWDRHEDDAPVLVNMYGITETTVHVTHRPLSRADLERPWSSTIGRPIPDLQVYVLDAARNPVPIGVSGEMYVGGAGVSRGYLGRSALTAERFVEDPFSARPGARLYRTGDLARWNSAGELEYLGRIDQQVKIRGFRIELGEIEAVLGEHPAVRACVVVAREDVPGNKRLVAYVVPDEGGVPTAAYREHLRAKLPEYMIPAAFVVLDALPSTPSGKVDRRALPAPEQRPEDGC

SEQ ID NO: 40 - DNA, 195 bp, Chondromyces crocatus
gcgctgctcgccgagatctggggcgggctgctcggcatcgagcgcgtcggcgcagaggacgacttcttcgcgctcggcggtcactcgctgctggccacacaagcgatctcgcgcatccgtgccgcgttcggcgtcgatcttcccctgcggacgctgttcgaggcgccgaccgtggcggagctcgcggcgaggatc

SEQ ID NO: 41 - protein, 65 aa, Chondromyces crocatus
ALLAEIWGGLLGIERVGAEDDFFALGGHSLLATQAISRIRAAFGVDLPLRTLFEAPTVAELAARI

SEQ ID NO: 42 - DNA, 1284 bp, Chondromyces crocatus
ggcgccgcgctgcccctgtccttcgctcaggagcggctgtggttcctcgacaggctggagccgaactgcgccttctacaacatcgccacggccttccacctcgcggggcccctcgatggggaagcgctcgcgcggagcctccgggagatcgtgcgccggcacgaggcgctgcgaacgacgttccctgcccgtgaaggtcaagctcaccaggtgatcggcgaggccgcgcgctggaccctgacgcacgcagacgtccagccgtcggagtggcgccgccgcatcgaggaggaggcccgtgcgcccttcgatctcgcggcgggcccgctcttccgggcgacgctcctgcgcgtgtcggacgtggagcacgtgctgctcctgacgatgcaccacatcgtcagcgatggctggtcgatgggcaccctcgcgcgtgagctggaagccctctacggtgccttcgccgccgggcggtcctcccccctggccgagctgccggtccaggtggccgaccacgccgtctggcagcggagccggctacgagggagaggcttcgaggcgcacctggcctactggcaggccaagctcgccggcgcgcagcctctcgtcctgccgacggatcgcccgaggccgccggccgcgtcgcaccagggtcgtctgctgaccttccagctcccccgagcgctcgcggtcgagcttcgcgcgctgagccgcaaggagggggcgacgctgttcatgaccttgctctcggccttcgcggtgctcctcgcgcgccacgcgaaccaggtcgacttctgcatcgggacgccgatcgccacgcggaaccgggaggcgctcgaagggctgatcggtctcttcgtcgacacgctcgtcctgcgggccgacctctcgggtgatccgaccttccgtgcgctcctcggacgcatgcgggacgaggcgctggcgagccacgcccaccaggaggtccccttcgagcgcatcgccgacaggctgggggtggcgcggagcctcggccagagcccggtgttccaggtgatgttcgcgctgcagaacgcgccgatggacgggctccgtctgccaggggtcgaggtgacctccgaggaggtggagacggggacctcgaagttcgatctctcgctctcgatgcaggagcatgccgaggggctcgtcggcgtgttcgaggtcgcgacggacctgttcgacgtctcgaccgtcgagcgcctcatcggtcagttcggcgtcctcttgcgcgcggtggtgcgtgacccggaggtgccagtgtccacgctg

SEQ ID NO: 43 - protein, 428 aa, Chondromyces crocatus
GAALPLSFAQERLWFLDRLEPNCAFYNIATAFHLAGPLDGEALARSLREIVRRHEALRTTFPAREGQAHQVIGEAARWTLTHADVQPSEWRRRIEEEARAPFDLAAGPLFRATLLRVSDVEHVLLLTMHHIVSDGWSMGTLARELEALYGAFAAGRSSPLAELPVQVADHAVWQRSRLRGRGFEAHLAYWQAKLAGAQPLVLPTDRPRPPAASHQGRLLTFQLPRALAVELRALSRKEGATLFMTLLSAFAVLLARHANQVDFCIGTPIATRNREALEGLIGLFVDTLVLRADLSGDPTFRALLGRMRDEALASHAHQEVPFERIADRLGVARSLGQSPVFQVMFALQNAPMDGLRLPGVEVTSEEVETGTSKFDLSLSMQEHAEGLVGVFEVATDLFDVSTVERLIGQFGVLLRAVVRDPEVPVSTL

SEQ ID NO: 44 - DNA, 1245 bp, Chondromyces crocatus
gccgagcgccaccagtcgctcgtgacgtggaacgacacggcgacggctgccccgcaggatcggtgcgttcacgcgctgttcatggagcgagcggcgaggacacctggcgccctcgcggtgatccacggcgaccggcagctcacctacgccgagctcgatgctcgctccagccagctcgcgcaccacctgcgagcgcggggagtcggccccgggacgctggtggcgctctgcgtcggccgctccgtcgatctgatcgtgggcgcgctcggcgcgctgaaagcagggggagcctacgtccctctggacccggcccatccagcggagcggctggcgttcatgctggaggacacgggcgcgaccgtgctgctgacccaggcagccctcgtggcacggctccccccgcacggcgcgcaggtcgtgctcctcgacgccgacgacgcgaccctcgacgcgtggcccgacgtggcgccgcccctgcgtacgacgtcggaggatctcgcttacgtcatctacacctcgggctcgacgggccggccgaagggcgtcctgctctcgcacgggggcctcgtgaacctctgcacgtggcacgtgggggcgtaccagctctctccagaagatcgcacgacgctgatcgcagcgccggggttcgacgcctcggtgtgggagatctggccagcgctgatcgcgggcgcctcgctgctgatcgtggacgacgagatccgcctgtcgccagccgcgctggcggacttcctcgtcacgcgcgaggtgacggtgaccttcctcccgacaccgctcgcggaggcgttgctgaccctcccctgggccacgggtggcgcgctgcgcgcggtgctgacgggcggagacgtcctgcggcgaaccccacccgcggcgctgcccttcgcgctcgtgaaccattacggaccgacggagtgcaccgtcgtggcgacggcggccgtggtcgtgccgggggggcagggggcgccaccgatcgggaagccgatcgcgaacgcccgggtgtacgtgctggatgcgcgcggcgcgcccgtgcccgtcggtgtccctggcgagctgtacatcggcggcgccggcctcgcccagggctacgcgaaccggccggcgctgacggcagagcggttcgtccccgaccccttcggcgacaccccggggcgtctctatcgcacgggggatctcgtgcggtggctgcccgacgggagcctcgcgttcctcggcgcatcgacgaccagg

SEQ ID NO: 45 - protein, 415 aa, Chondromyces crocatus
AERHQSLVTWNDTATAAPQDRCVHALFMERAARTPGALAVIHGDRQLTYAELDARSSQLAHHLRARGVGPGTLVALCVGRSVDLIVGALGALKAGGAYVPLDPAHPAERLAFMLEDTGATVLLTQAALVARLPPHGAQVVLLDADDATLDAWPDVAPPLRTTSEDLAYVIYTSGSTGRPKGVLLSHGGLVNLCTWHVGAYQLSPEDRTTLIAAPGFDASVWEIWPALIAGASLLIVDDEIRLSPAALADFLVTREVTVTFLPTPLAEALLTLPWATGGALRAVLTGGDVLRRTPPAALPFALVNHYGPTECTVVATAAVVVPGGQGAPPIGKPIANARVYVLDARGAPVPVGVPGELYIGGAGLAQGYANRPALTAERFVPDPFGDTPGRLYRTGDLVRWLPDGSLAFLGASTTR

SEQ ID NO: 46 - DNA, 195 bp, Chondromyces crocatus
gcgatgctggccgagatctggagccgcttgctcggggtcggccaggtcggcgcgcaggacgacttcttcgcgctgggcggccactcgctgctcgcgacgcaggtcgtctcgcgcatccgcgcggccttcggggtggagctgcccctgcgcgcgctcttcgaggccccgaccgtggcggggctcgcggcgcgcctc

SEQ ID NO: 47 - protein, 65 aa, Chondromyces crocatus
AMLAEIWSRLLGVGQVGAQDDFFALGGHSLLATQVVSRIRAAFGVELPLRALFEAPTVAGLAARL

SEQ ID NO: 48 - DNA, 1284 bp, Chondromyces crocatus
ttcctgcagcggatggatggccccggcgccacctaccacatccccttcgccctgcacttccagggggagctggacctgccggccttgcaggctgcggtcggcgacgtcatggcccggcacgagagcttgcggaccgtgttccccgtcgtcgacgaggtgcctcaccagcgcatcctcgacgtggacgccgcgcccctccggtggaccgtcacgccggcggcccccgccgcgctgcccgggctgctgaccgaggcgacccagcggggcttcgatctggcggtcgagcctccgctgcgcgcggaggtgttctcgctcggccccgacgaccacgtgctcttgctcctgctccaccacatcgccggtgacggctggtcgatggggcccctgcgcgcggatctcaccgccgcgtacctggcgcgccgtcagggcaaggctcctggctggagcgcgcttcccgtgcagtacgccgactacaccctgtggcagcaccggctcctcggcgagcagcgcgatccggacagcctgttcgccacccagctcgcgtactggacccggaccctcgccggcctcccggagcagctcccgctgcccgccgatcgtcctcgcccggcggtggcctctcaccggggtggcgtcgtcccgttccggctgggaccggccttgcacgaggggctcctcgacctcgctgcgcaggggggcgccagcctgttcatggtgctgcaggccggcctggctgcgctcctgtcgcggctcggtgcaggggacgacatcgtggtggggagcccgatcgccggacgcaccgaccacgccctcgaccacctcgtcgggttcttcgtgaacacgctggtgctgcgcaccgacacctcgggagatcccagcttcctccagctcctcggccgggtgcgcgaggccgccctcggggcttacgcccaccaggacgtgccgttcgagtacctggtcgaggtcctgaaccccgtccgctcgctgtcccaccaccccctgttccaggtgatgctggtgctgcagagccaccaggacgacggcatcgacctgcccgggctgcgcgtggctgcgatgccggtctcgctggagaccgccaagttcgatctgctgttcgcgctgagcgagcggcgcggggcggatggtgcccgcgagggcctcgacggcgtgatcgagtacgccagcgatcggttcgaccccgggaccgtcgaggggatcgtggcgcggtggctccgcctgctcgaggctgccgtggccgatcccgggctgccgatccgacggatc

SEQ ID NO: 49 - protein, 428 aa, Chondromyces crocatus
FLQRMDGPGATYHIPFALHFQGELDLPALQAAVGDVMARHESLRTVFPVVDEVPHQRILDVDAAPLRWTVTPAAPAALPGLLTEATQRGFDLAVEPPLRAEVFSLGPDDHVLLLLLHHIAGDGWSMGPLRADLTAAYLARRQGKAPGWSALPVQYADYTLWQHRLLGEQRDPDSLFATQLAYWTRTLAGLPEQLPLPADRPRPAVASHRGGVVPFRLGPALHEGLLDLAAQGGASLFMVLQAGLAALLSRLGAGDDIVVGSPIAGRTDHALDHLVGFFVNTLVLRTDTSGDPSFLQLLGRVREAALGAYAHQDVPFEYLVEVLNPVRSLSHHPLFQVMLVLQSHQDDGIDLPGLRVAAMPVSLETAKFDLLFALSERRGADGAREGLDGVIEYASDRFDPGTVEGIVARWLRLLEAAVADPGLPIRRI

SEQ ID NO: 50 - DNA, 1590 bp, Chondromyces crocatus
gacacgtacaacgacaccgcccgccccgtccccgagaccagcttgcccgcgctgttcgaggcgcaggccaagatggcacctgcgcgcccggccctggtgttcgaggacgccgtgctgacgtacgccgagatcaacgcccgcgccaaccgcctggcgcacgtgctgatcgcgcagggggtcggcccggagcgcatcgtcgccttgctcttgccgcgcacccccgagctgatcgtcgcgctcctggcgacgctcaagacgggggccgcctacctgcccgtggacccggagtaccccgcgtcacggatcgcgacgatgctgagcgacgcccgccctgcggtcgtgctggcgagcctggagactgcgcgcgcgatccccgagggcatcacgttcccctgcctggtggtggacgagcccgacacggctgccgcggtgtcccgtcatcgcgccaccgacccgacggacgtcgagcgcaccgttgccttgatgccgcagcatccggcgtacgtgatctacacgtccggatcgaccggcatccccaagggcgtggtcatgccctccggcgccctggtgaacctgctgttctggcaccagcgcgccttgccgagcggcgagggcacccgcgtcgcgcagttcacggccctgagcttcgacgtctcggcgcaagagatcctctccacgctgctcttcgggaagaccctggtcgtgccgccggacgccgtgcggcgcagcgcggagcggctggcgggctggctcgcgaagcaccgcgtcgaggagctgttcgctccaaacctcgtcgtggaagcgctggccgaggccgccctcgagcgaggcctcaccttgccccatctgcgcgacatcgcgcaggcaggcgaagcgctcaccctgagtcgccacgtgcgcgagttccaccgtcgaacgcccggccgccgcctgcacaaccactacggtccggcggagacacacgtggccaccggctgcacgctgcccgccgatctcgcgacctgcacgctgccgccgtccatcggccagccgatcttcaacacgcgcgtgtacgtgctggatgaccggctggacctgacgcctgccggcatcgcaggggagctgtacctcaccggggccgggctcgcgcgaggctacctggaccggcctggcttgacggcccagcggttcatccccgaccccttcggccccccgggcgcgcgcatgtaccgcaccggagaccaggcgcggtggcgcgcagcgggggagctggagttcctcggccgcctcgaccaccaggtcaagatccggggcttccgcatcgagctgggcgagatcgaggcggtgctggccgcgcatcccgagctttctcgggcggcggtcctcgcccgcgatcaccagtcgggagggaagtggctggtggcctacgtcgtccctgtgccgcacgctgccccgcggcccgaggccttgcgcgagcacctgcgccagcggctccccgattacatggtccccggggccgtggtggtcctggagcgcctccccctgacgctgaacgggaagctcgatcgccaggcgctgcctgcgccggagctgagcccggaacgggcgggg

SEQ ID NO: 51 - protein, 530 aa, Chondromyces crocatus
DTYNDTARPVPETSLPALFEAQAKMAPARPALVFEDAVLTYAEINARANRLAHVLIAQGVGPERIVALLLPRTPELIVALLATLKTGAAYLPVDPEYPASRIATMLSDARPAVVLASLETARAIPEGITFPCLVVDEPDTAAAVSRHRATDPTDVERTVALMPQHPAYVIYTSGSTGIPKGVVMPSGALVNLLFWHQRALPSGEGTRVAQFTALSFDVSAQEILSTLLFGKTLVVPPDAVRRSAERLAGWLAKHRVEELFAPNLVVEALAEAALERGLTLPHLRDIAQAGEALTLSRHVREFHRRTPGRRLHNHYGPAETHVATGCTLPADLATCTLPPSIGQPIFNTRVYVLDDRLDLTPAGIAGELYLTGAGLARGYLDRPGLTAQRFIPDPFGPPGARMYRTGDQARWRAAGELEFLGRLDHQVKIRGFRIELGEIEAVLAAHPELSRAAVLARDHQSGGKWLVAYVVPVPHAAPRPEALREHLRQRLPDYMVPGAVVVLERLPLTLNGKLDRQALPAPELSPERAG

SEQ ID NO: 52 - DNA, 195 bp, Chondromyces crocatus
cagctgctgtgcgacctgttcgccgaggtgctggggctggggcaggtgggcatcgatgaggacttcttcgaactgggcggtcactcgctgctggcgacgcggttgatcggccggatccgcgccaccctgggcgtggaggtgccgctccaggcgctgttcgaagccccgacggtggccggcctctcgacgcagctc

SEQ ID NO: 53 - protein, 65 aa, Chondromyces crocatus
QLLCDLFAEVLGLGQVGIDEDFFELGGHSLLATRLIGRIRATLGVEVPLQALFEAPTVAGLSTQL

SEQ ID NO: 54 - DNA, 1329 bp, Chondromyces crocatus
ccggacgcgctgccgctgtcgttcgcgcagcagaggctgtggttcctgcaccagatggagggccgcaccgcgacgtacaacatgccgctggcgctgcgcctgaccggtgcgctcgaccggacggccctccagacggccctgggtgacgtgatcacgcgccacgagagcctgcggacggtgttcccgcaggtggaagggatgcctttccaggtggtcctcgacgccgacaaggcgcgtcctgtgttgaccctcctccggaccgacgagaagggcctgcgcgaggcgctggccaccgcagcccgacacggcttcgacctgtccgtcgagccaccgctgcgggccacgctgttcgaggtggcgcccgaggtccacgtgctgctgctgacgatgcaccacatcgtcggcgacggctggtccatggggcccctctcgcgcgacctcgccgctgcctatgccgcgcgctgccagggggaagcgccggcctggtcgccgcttccggtgcagtatgccgactacacgctctggcaacgggagctgctcggcgaccaggccgacgccgagagccggttcgcgcagcagctcgcctactggaccagaaccctcgccgacctccccgagcagctggagctgcccaccgatcgcccacgcccgccggtggcctcctaccagggcagcgtgctcccggtgacctgggacgcgcacctgcatcagggcctcgccgatctcgcccgccagagcggcgccagcttgttcatggtgctccaggccggcctcgccgccttgttcacgcgcctgggcgcaggccatgacgtcgccctgggcagccccatcgcgggtcgcaccgatcccgcgctcgacgacctggtcgggttcttcgtcaacacgctggtgctgcgcacggacacgtcggggaacccgagcttccggcagctcctgggccgcgttcgtgaaacggccctggccgcctatgcccatcaggacgtgccgttcgagttcctggtcgaggcgctgaacccggcgcggtcgatggcccatcaccccctgttccaggtcatgctcggcgtccagaacgcgcccgcgggcgccttccagcttcccggactgcacgtggaaccgatgggcacgggcggtacggagacctcacgcgtcgacctgacgttcagcgtcaccgagcgccgcaccgccgagggcgccgcggaaggcatcgagggggtggtcgagtacagcagcgacctgttcgacgccgccacggtcgaggcgctggtggcacggtgggcgcggctgctggaggccgccgtcgcggacccggatcagcccatcgggagcctg

SEQ ID NO: 55 - protein, 443 aa, Chondromyces crocatus
PDALPLSFAQQRLWFLHQMEGRTATYNMPLALRLTGALDRTALQTALGDVITRHESLRTVFPQVEGMPFQVVLDADKARPVLTLLRTDEKGLREALATAARHGFDLSVEPPLRATLFEVAPEVHVLLLTMHHIVGDGWSMGPLSRDLAAAYAARCQGEAPAWSPLPVQYADYTLWQRELLGDQADAESRFAQQLAYWTRTLADLPEQLELPTDRPRPPVASYQGSVLPVTWDAHLHQGLADLARQSGASLFMVLQAGLAALFTRLGAGHDVALGSPIAGRTDPALDDLVGFFVNTLVLRTDTSGNPSFRQLLGRVRETALAAYAHQDVPFEFLVEALNPARSMAHHPLFQVMLGVQNAPAGAFQLPGLHVEPMGTGGTETSRVDLTFSVTERRTAEGAAEGIEGVVEYSSDLFDAATVEALVARWARLLEAAVADPDQPIGSL

SEQ ID NO: 56 - DNA, 1569 bp, Chondromyces crocatus
atcctgacggccgaagagcgccagaagctgctggtcgaccacaacgccacggcccatccggtcgcggccatcagcctgagcgcagcgttccaggcgcaggtggaggcaacgccggacgcggtggcggtggtgtgcgacggcacggcgctgacgtacgccgagctgaacgcgcgggcgaaccgactggcgcaccggctgacggcgcatggggtgtcaccggagagccgtgtggcgctggtgctggagcgctcgctggagctggtggtgggcttgctgggggtgatcaaggccggtggcgcgtacgtgccgctggacgcgcgctacccgcaggcgcggagagcgcacatcctgaaggaaacgggcgcggtcgtgctgctggccagcggggaggggagcgaggacaccgcgtcgctgggcatcccggtgctggtggtcgatgctggacccgtggtctccgatccgggctccccggccgcggactccgatccggaccagctcgcgtacgtcatgtacacgtcggggtcgacggggcagccgaaggggatcggtgtcacgcaccggaacgtggtggagctggcctcggatccatgctggcgctcggggcatcatcgtcgggtgctgtggcattcccctccggcgttcgacgcctcgacgtacgagttctgggtgcctctgctgggtggcgggcagatcgtcgtcgctcccgccggggagcagaccgcccacgacctgaggcgtgtgctccgtgaacatcgggtcaccagcgtcttcctgacgacggcgctgttcaacctgatggtggaggaagacccgagcagcttccgcacggtgggcgaagtgtggaccggcggcgaggccgtctcgcctcaggcgatgcagcgggtgctggatgcctgtccggacacgatgatcgcccacgtctacggcccgacggagacgacgacgttcgccacgttcgaggccctgcgaccgccgcaccacatcgagggcacggtgccgatcggcaagccgatggcgaacatgcgggcctacgtgctcgacgaaggattgcggcccgtgccagaaggcgtgcccggggagctgtacctcgcgggcgccgggctctcgcgcggatacgtcgcgcgctccgggctgacggccgagcgcttcgtcgtcgacccgttcgccagcggcgagcgcatgtaccgcaccggcgatcgtgtccggtggaacgccgacgggagcctcgacttcctgggccgcaccgacaaccaggtgaagatccgaggcttccgcatcgagccggacgagatcggcacggtgctgctggagcatcccgaggtcgcgcaggcggcggtcgtcgtgcgcgaggaccggcctggcgagaagcagctgatcgcttacgccgtcgccaccgcggaaacttctcccgacccgcgtgcgctgcgcgactggctcaagcaccgcctgcccgagtacatggtgcccgccgcgctcgtcctgctcgacgccttgccgctgaacgcgaacggcaagctcgaccgcaaggcgctccccgcacccgacctcggtcccacccgcgtcggccgg

SEQ ID NO: 57 - protein, 523 aa, Chondromyces crocatus
ILTAEERQKLLVDHNATAHPVAAISLSAAFQAQVEATPDAVAVVCDGTALTYAELNARANRLAHRLTAHGVSPESRVALVLERSLELVVGLLGVIKAGGAYVPLDARYPQARRAHILKETGAVVLLASGEGSEDTASLGIPVLVVDAGPVVSDPGSPAADSDPDQLAYVMYTSGSTGQPKGIGVTHRNVVELASDPCWRSGHHRRVLWHSPPAFDASTYEFWVPLLGGGQIVVAPAGEQTAHDLRRVLREHRVTSVFLTTALFNLMVEEDPSSFRTVGEVWTGGEAVSPQAMQRVLDACPDTMIAHVYGPTETTTFATFEALRPPHHIEGTVPIGKPMANMRAYVLDEGLRPVPEGVPGELYLAGAGLSRGYVARSGLTAERFVVDPFASGERMYRTGDRVRWNADGSLDFLGRTDNQVKIRGFRIEPDEIGTVLLEHPEVAQAAVVVREDRPGEKQLIAYAVATAETSPDPRALRDWLKHRLPEYMVPAALVLLDALPLNANGKLDRKALPAPDLGPTRVGR

SEQ ID NO: 58 - DNA, 195 bp, Chondromyces crocatus
cacctgctctgcgacctcttcgccgagatcctcggcctgccacgcgtcggcatcgacgacgacttcttcgagctgggcggccactcgctgctcgccacccgcctcgtcagccgcgtgcgctccaccctcggcgtcgacatgggtctgcgccgcctgttcgaggcgcccaccgtcgctgggctcgcagcctgcctc

SEQ ID NO: 59 - protein, 65 aa, Chondromyces crocatus
HLLCDLFAEILGLPRVGIDDDFFELGGHSLLATRLVSRVRSTLGVDMGLRRLFEAPTVAGLAACL

SEQ ID NO: 60 - DNA, 732 bp, Chondromyces crocatus
cccccgctcttctgcatgcacccgggtggtggcatgagctggagctacgccggcctgatgcgccacctcgacccggagacgcccctctacggcatccaggcgcgcagcctcgctcgacccgagccgcgcccgacctccctccaggccatggccagcgactacgccgaccagctccagcggatccagcctctgggaccctaccacctcctcggctggtcctccggcggcctcgtcgctcacgccgtcgccaccgagctgcaacggcgtggcgccgaggtggcgctgctcgccctcctcgacgcctatcccctggtcgacatcgccctcgacgagcccctggtgcagagcgaacgcgccatcctcgccgggatgatcgaagccgacccgagcgacctgcagggcatggatgaccagcaagcggtcacgcacgtcctcgaagtcctccgccaccagggcaacgtgctggccagcctcgacgcgcgccagatccgcaccctcatcgacctcatgacccacaacgccggcctcgtctccgacttcgtccctgccgtgtaccagggcgacctggtgctcttcagcgccaccatcaaccgcccagatccggcgcgaccggcgctctggcagccctacgtcagcggcgccatcgagaaccatgacatcgagatccgtcacgaccacatgatgcagcccgcgccgctcgcccagatcgggcgcatcgtcgcggccaggcta

SEQ ID NO: 61 - protein, 244 aa, Chondromyces crocatus
PPLFCMHPGGGMSWSYAGLMRHLDPETPLYGIQARSLARPEPRPTSLQAMASDYADQLQRIQPLGPYHLLGWSSGGLVAHAVATELQRRGAEVALLALLDAYPLVDIALDEPLVQSERAILAGMIEADPSDLQGMDDQQAVTHVLEVLRHQGNVLASLDARQIRTLIDLMTHNAGLVSDFVPAVYQGDLVLFSATINRPDPARPALWQPYVSGAIENHDIEIRHDHMMQPAPLAQIGRIVAARL

SEQ ID NO: 62 - DNA, 1836 bp, Chondromyces crocatus
gtgggtcttcaagatggagatcgaaccccgggtggggggccacgtctcccgcggccctggcaaggtgaccgtctgggaacccccgcggcacttcggctacctccacagcgtggaagggttctcggcacgcctcgagtaccgtgtcgaggccgccggggaaggcagcgtgctgcacacctggatccaccgcgagtacggcaagcccatcgacgacctcgacttccagcgcgagctcgccgagacgcacacctggttctacaaccacacgctcgggcagtacctggcccacttcaacgggcgccccgcgaccttcgtggagcttctcggacccgaggcctcacggacggcggaagcctcggccacgctccgccgacggctcggcctgagcgacgacctcaccgaaggcgacaccgtgagcgccaccttcccggacgtggggacggtggaggccgtcgtggactacgtcaggcctcagttcctgggcctccgcaccgaggcgggcctctaccgcttcttctgcaggaacgcctggggatggcccatcggcatgagcctccacctcttcgccgagggcaccgatgcggagaagaccgagcatgcgtggcgcgcctggctggacgggacgttcgccggatgagcgaccccaccttcgccgaggcgatggccacgccggcgttccaccaggacccgtacccgctctacgcccgccttcgcgacgagcagccgctgtaccgcagcccgcacggcgtcgcatacctgagccgctacgccgacgtcgacagggccctgcgcgacccgcgcctgtccaacgatcgcgagcggatcatccgcgccatgaccccgccggacggcgagacgcccctcatcgcccgcctcatgcgcaagctggggcgggtgatgaccaacaccgatccccccgcccacgcgcgtctgcgcaagctcgtcggcaaggccttcggcgcgggttggatccgcgacttccggccgcgtatccagtcgctcaccgacgcgctgctcgacaccatgtgcgcggccggagcgcgcatggatctcatcgcgtccctggcctacccgctgaccagcaccgtgatctgcgagctgctcggcgtcccgcgcagcgaccaggagcgcaccctcgagtggctgcgtcagctcgagaacccgacggctgccggcctctcgatcgaggagaccgagcaggtggtcgacgcgctctacggcgagctgcgcgcgctcatccaccgccggcgcgcagcgcccgaagacgatgtcctcagcgccctgagccaggtggaggacggcggcgaccggctcgacgacgacgagatgctctccgcctgcttcgtgctgattggctccggctacgagaccaccatgaacctgatcgcgaacagcgtgctcacgctgctgcgccaccccgagcagctccgcgcgttgcacgagaagcccgagctgctccagcctgccatcgaggaggtgctgcgctacgagagcccgtccctgcaggtgatccgcgtcgtcgccgatccggtggagatcgcgggtggcacgttgcgcgagggcgagatggtcaccctcctcctcggctccgccaaccgcgacccgcttcgcttcccccacccggagcgcttcgacatcacccgcggcgacagccgccacgtgagcttcggcagcggcattcatttctgcctcggcgcacccctggcccggctggaggcctccgtcgccctgagcacgctgctccggcgcttcccgacgctgcgcctcgatgaagagggggtggaatggagggcgaacccctcgctgcgcgggctggctcgcctggtggtcgcctgg

SEQ ID NO: 63 - protein, 612 aa, Chondromyces crocatus
VGLQDGDRTPGGGPRLPRPWQGDRLGTPAALRLPPQRGRVLGTPRVPCRGRRGRQRAAHLDPPRVRQAHRRPRLPARARRDAHLVLQPHARAVPGPLQRAPRDLRGASRTRGLTDGGSLGHAPPTARPERRPHRRRHRERHLPGRGDGGGRRGLRQASVPGPPHRGGPLPLLLQERLGMAHRHEPPPLRRGHRCGEDRACVARLAGRDVRRMSDPTFAEAMATPAFHQDPYPLYARLRDEQPLYRSPHGVAYLSRYADVDRALRDPRLSNDRERIIRAMTPPDGETPLIARLMRKLGRVMTNTDPPAHARLRKLVGKAFGAGWIRDFRPRIQSLTDALLDTMCAAGARMDLIASLAYPLTSTVICELLGVPRSDQERTLEWLRQLENPTAAGLSIEETEQVVDALYGELRALIHRRRAAPEDDVLSALSQVEDGGDRLDDDEMLSACFVLIGSGYETTMNLIANSVLTLLRHPEQLRALHEKPELLQPAIEEVLRYESPSLQVIRVVADPVEIAGGTLREGEMVTLLLGSANRDPLRFPHPERFDITRGDSRHVSFGSGIHFCLGAPLARLEASVALSTLLRRFPTLRLDEEGVEWRANPSLRGLARLVVAW

SEQ ID NO: 64 - DNA, 19 bp, Pseudomonas aeruginosa
gataatgataatcattatc

SEQ ID NO: 65 - DNA, 19 bp, Pseudomonas putida
aaacatgaaggacatgttc

SEQ ID NO: 66 - DNA, 19 bp, Pseudomonas putida
aataatgaatatcattatc

SEQ ID NO: 67 - DNA, 19 bp, Pseudomonas putida
aataacaagaattaatact

SEQ ID NO: 68 - DNA, 19 bp, Pseudomonas putida
cataatgcgcggcgatatc

SEQ ID NO: 69 - DNA, 939 bp, Pseudomonas putida
atcaggccgcgctgattcgccgtatggggcgcgggctgctggtgaccgaactgatggggcatggcttgaacatggtgacgggggactattcccgtggtgcggcggggttctgggtcgagaatggcgagattcagcatgccgtacaggaagtcaccatcgccggaaacatgaaggacatgttccagcagattgtcgcgatcggtagcgatcttgaaacccgtagcaatattcatacgggctcggtgttgatcgagcggatgaccgttgctggtagctgatctttagcctgcgccggccctttcgcgggtaaacccgctcctacacggtggtggacgtacatcggggttggacacaggccgttgtaggagcgggttcacccgcgaagaggccggaacagcactacacctttccctgcaaatccgaagacccggccctcgcgccgggtttttatttcatcacctttttcttgaagtgattctatttatcacttaataatgaatatcattatccagtaacccggcgatgatgttcatgaaatccgtcctccgcgaactgccctacctggaaaactggcgctggctcagccggcgcattcgctgtgcgctcgaccccgacgagccgcgcctgatcgagcattacctggccgaaggccgctatctggtgtgctgcaccgaaacctcgccatggacggtggcgctgacagcgtttcgcctgctgctggataccgcctgcgatcgcatgctcccctggcattggcgttgtctgtgcctggaccaggcgtggcgccctctgctggacctgcgcaacctcgaccgccaggaacagaaccaacgctggcaaccctacgccttgcagttggccaattgccgtctgctgccttcgatttctcccgatgaactgatgcaaggatttgatgatgagtgatacccgtatcgagcg

SEQ ID NO: 70 - DNA, 250 bp, Pseudomonas putida
tccggcgaattttctacacagagctgctgccggacctcaagcgcctgggcaagaccatcatcgtgataagccacgacgaccgctacttcgacgtcgccgaccagctcatccacatggcggcaggcaaggtccaacaggagaaccgcgtcgcagattgcatttaatttttccggttttggccgatgagtgcgtcccaatcaataacaagaattaatactattaacatctgacactcaagggctttgaaaaa

SEQ ID NO: 71 - DNA, 400 bp, Pseudomonas putida
caggtagcgcaggcgctcttccaggtggcgcaactgagtgtcgtcaaggctaccggtcacttccttgcgatagcgggcgatgaagggcacggtcgagccttcgtccaacaggctcacggccgcctcgacctgctgcgggcgtacgcccagttcctcggcgatacggctgttgatgctgtccatgtaaaccacctgacatttgtgaatacgggggtcgcctgtgggctttttgcccggcggcgctggatgaaagccgcgcattatacccatcgcaaacggcttgcggtgatggcgcccggccagccggaactggcgccgggggaaaaatctgctaacaatgctcacgcaacgtgcagcaatggctacgccataatgcgcggcgatatcagaggagttattc

(19) United States
(12) Patent Application Publication
(10) Pub. No.: US 2012/0034650 A1
(43) Pub. Date: Feb. 9, 2012

(54) Nucleic acid molecule of a biosynthetic cluster encoding non ribosomal peptide synthases and uses thereof

(75) Inventors: Philipp Krastel, Grenzach-Wyhlen (DE); Brigitta-Maria Liechty, Basel (CH); Charles Moore, Basel (CH); Esther Schmitt, Lorrach (DE)

(73) Assignee: NOVARTIS AG, Basel (CH)

(21) Appl. No.: 13/148,520

(22) PCT Filed: Feb. 11, 2010
(86) PCT No.: PCT/EP2010/051696

§ 371 Date: Oct. 25, 2011

Related U.S. Application Data
(60) Provisional application No. 61/152,517, filed on Feb. 13, 2009.
(60) Provisional application No. 61/219,940, filed on Jun. 24, 2009.

(51) Int. Cl. C12P 21/00; C12N 9/16; C12N 9/02; C12N 9/00; C12N 1/21; C12P 21/02; C12N 15/52; C12N 15/55; C12N 15/63

(52) U.S. Cl. 435/68.1; 536/23.2; 435/196; 435/189; 435/320.1; 435/252.3; 435/252.34; 435/252.35; 435/183

(57) ABSTRACT

The present invention relates to the provision of a polynucleotide comprising one or more functional fragments of a biosynthetic gene cluster involved in the production of a compound of formula (I) or (I′). The present invention also provides a method of preparing a compound of formula (I) or (I′) or of formula (II) to (VII), (XI) to (XIV) and (XVII) and (XVIII). Moreover, the use of such compound as a pharmaceutical composition is also provided in the present invention.

The present invention relates to the provision of a polynucleotide comprising one or more functional fragments of a biosynthetic gene cluster involved in the production of a compound of formula (I) or (I′). The present invention also provides a method of preparing a compound of formula (I) or (I′) or of formula (II) to (VII), (XI) to (XIV) and (XVII) and (XVIII). Moreover, the use of such compound as a pharmaceutical composition is also provided in the present invention.

Many natural products derived from microorganisms possess biological activities observable in higher organisms and have been exploited for their therapeutic properties for centuries. Most of these natural products belong to the polyketide and non-ribosomal peptide classes and are synthesized by modular enzymatic systems known as polyketide synthases (PKS) and nonribosomal peptide synthases (NRPS) (Finkering and Marahiel 2004; Staunton and Weissman, 2001). In addition, pathways exist that contain both PKS and NRPS genes in the same pathway and thus produce secondary metabolites that are hybrids of these two classes. The natural products produced by these biosynthetic pathways are constructed from small, relatively simple building blocks such as short chain carboxylic acids and amino acids. However, the final natural products derived from these pathways are extremely diverse and often structurally complex, usually containing multiple stereocenters. For these reasons, synthetic approaches to the production of these compounds are often impractical and therefore fermentation remains the customary approach to their production. However, fermentation processes have inherent problems related to their reliance on microorganisms that are not metabolically characterized, often genetically intractable and frequently grow poorly and produce their compounds of interest at insufficient levels. To circumvent these problems, heterologous expression of the PKS or NRPS pathway in a well characterized host organism that does not have these drawbacks can be an option (reviewed by Wenzel and Muller, 2005). In fact, this approach can be extended to express “silent” or “cryptic” PKS and NRPS pathways for discovery efforts (Shen, 2004) or used to express pathways from organisms that are unable to be cultured in the laboratory. Furthermore, the transfer of PKS and NRPS pathways into heterologous hosts permits efficient bioengineering of secondary metabolite pathways to generate novel analogs of the parent compound.

Heterologous expression takes advantage of the fact that, in general, PKS and NRPS pathways are located in a contiguous cluster on the genome. Therefore, these pathways are, in principle, relatively easy to clone into standard BAC or cosmid vectors. Despite the topical simplicity of moving a pathway from one microorganism to another, differences in regulation, codon usage or metabolism between the two organisms pose significant challenges to successful heterologous expression. Furthermore, the molecular tools that allow this strategy to be efficiently applied such as BAC library construction and recombination approaches to cloning have only relatively recently become available (Wenzel and Muller, 2005). For these reasons only a few examples of successful heterologous expression exist in the literature.

The choice of a suitable heterologous host is an important consideration when designing an expression strategy. The new host should be genetically tractable, easy to handle in the laboratory and have the ability to employ PKS or NRPS pathways. For example, the presence of a phosphopantetheinyl transferase in the new host is essential to facilitate the activation of imported PKS or NRPS (Pfeifer et al. 2001). In addition, it is vital that the new host has a similar codon usage profile to that of the native host to permit efficient expression of the imported pathway. The most common hosts employed have been Escherichia coli, Bacillus subtilis, Pseudomonas putida and a small selection of well characterized Streptomyces strains (reviewed in Zhang and Pfeifer, 2008). Other hosts that have been utilized include Myxococcus xanthus and filamentous fungi. Some of these host strains have been modified such that the major indigenous secondary metabolism systems have been silenced via mutagenesis to remove background metabolite profiles and to prevent drawdown of the precursor pool available to the incoming biosynthetic pathway.

In order to transfer a particular pathway, the packaging of the pathway on a suitable transferable genetic element is required. The sequence of the PKS or NRPS system must initially be known, at least at the amino acid level, and more preferably at the nucleotide level. Typically this sequence is used to design a probe to locate a BAC or cosmid clone from a genomic library constructed from the native host. Due to the large size of these pathway clusters (usually greater than 30 kb and often over 100 kb) they are often not captured in a single BAC or cosmid clone when a “shotgun” cloning strategy is employed. Therefore, the pathway must often be reconstructed to generate a single BAC or cosmid vector construct that contains the entire pathway. When very large pathways are to be expressed they may be broken into two or more separate vector constructs to be expressed in trans in the new host (Gu, et al. 2007). Ultimately, the vector construct must also possess plasmid transferability functions (e.g. oriT from RK2) to move it from the E. coli harboring the construct into the new host. To ensure that the construct is stable in the new host it is advisable to integrate it into the host chromosome. To accomplish this, the construct must contain a site for efficient chromosomal integration. For example, the phage attachment site φC31 for Streptomyces is often utilized for chromosomal insertion in this system (Binz, et al. 2008). Furthermore, it is often necessary to insert a new promoter in front of the biosynthetic pathway that will function properly in the new host. If the two organisms in question are closely related, and therefore likely to share many regulatory elements in common, this step may be avoidable. Finally, a selectable marker, generally an antibiotic resistance cassette, is required to select for successful transfer and integration of the construct (modified BAC or cosmid) in the new host. Typically these manipulations are performed in E. coli and often through the employment of Red/ET recombination (Zhang, et al. 1998). This cloning approach is particularly amenable to applications involving large DNA constructs where restriction enzyme-based manipulations are challenging at best.

Once the construct has been integrated in the new host, fermentation and subsequent chemical analysis is performed to determine whether or not expression of the pathway has succeeded. When heterologous expression has succeeded in almost all cases the natural product has been produced at lower titers compared with those observed in the native host. Despite this obvious setback, successful heterologous expression provides an expression platform with many options available for traditional strain improvement methodologies.

The present invention relates to the identification of the biosynthetic cluster involved in the biosynthesis of the depsipeptides of formula I,

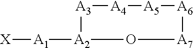

wherein the ester bond is found between the carboxy group of A7 and the hydroxy group of A2, and, optionally, the nitrogen atom of the amid bond between A5 and A6 is substituted with a methyl
wherein X and A₁ are each independently optional,
and wherein
   X is any chemical residue, particularly H or an acyl residue, particularly CH₃CH₂CH(CH₃)CO, (CH₃)₂CHCH₂CO or (CH₃)₂CHCO
   A₁ is a standard amino acid which is not aspartic acid, particularly glutamine;
   A₂ is threonine or serine, particularly threonine;
   A₃ is a non-basic standard amino acid or a non-basic derivative thereof, particularly leucine;
   A₄ is Ahp, dehydro-AHP, proline or a derivative thereof, particularly Ahp or a derivative thereof, particularly the Ahp derivative 3-amino-2 piperidone;
   A₅ is isoleucine or valine, particularly isoleucine;
   A₆ is tyrosine or a derivative thereof, particularly tyrosine;
   A₇ is leucine, isoleucine or valine, particularly isoleucine or valine, particularly isoleucine.

and the development of heterologous expression systems for the production of non ribosomal peptides of formula I including pharmaceutically acceptable salts or derivatives thereof. In particular, the biosynthetic gene cluster finds use in the biosynthesis of depsipeptides of formula (I′)

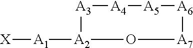

wherein the ester bond is found between the carboxy group of A7 and the hydroxy group of A2, and, optionally, the nitrogen atom of the amid bond between A5 and A6 is substituted with a methyl
, wherein
   X is CH₃CO, (CH₃)₂CHCO, CH₃S(O)CH₂CO, CH₃CH₂CH(CH₃)CO or C₆H₅CO
   A₁ is glutamine;
   A₂ is threonine;
   A₃ is leucine;
   A₄ is Ahp, dehydro-AHP, proline or 5-hydroxy-proline;
   A₅ is isoleucine or valine, particularly isoleucine;
   A₆ is tyrosine;
   A₇ is isoleucine or valine, particularly isoleucine.

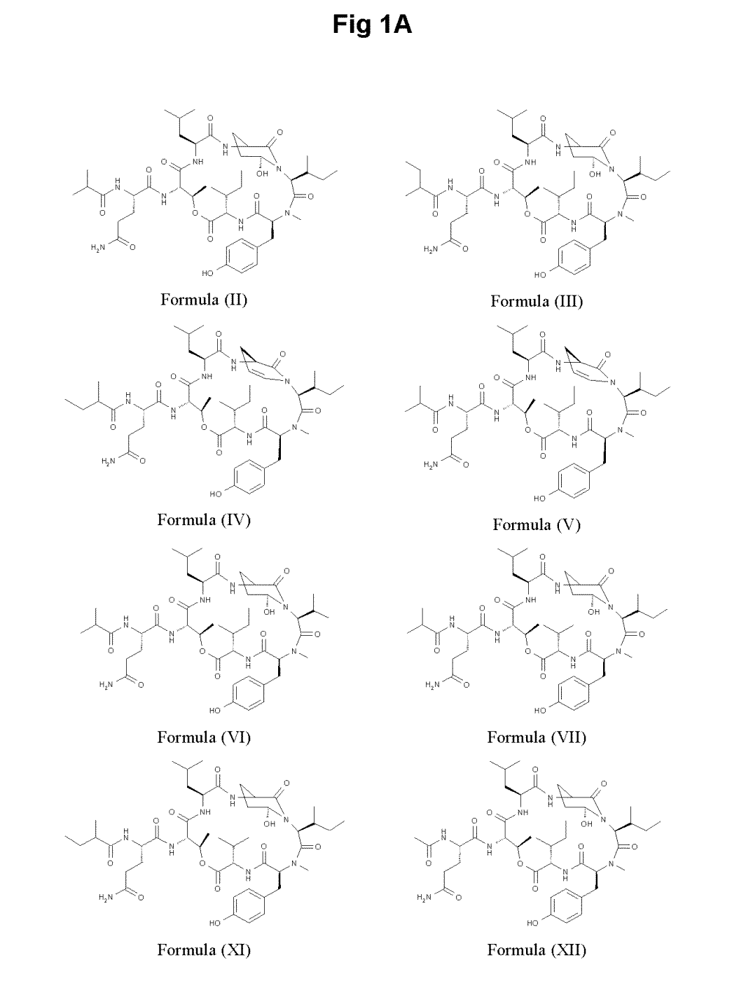

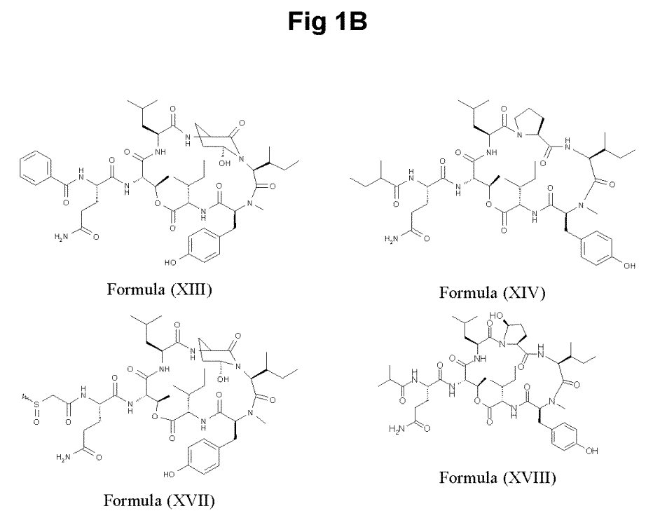

In particular, the present invention relates to the identification of the biosynthetic cluster involved in the biosynthesis of non ribosomal peptides of formula (II), (III), (IV), (V), (VI), (VII), (XI), (XII)-(XIV), (XVII) and/or (XVIII) as shown in FIG. 1 and the development of heterologous expression systems for the production of non ribosomal peptides of formula (I) or (I′) including pharmaceutically acceptable salts or derivatives thereof.

Compounds of formula (I), in particular of formula (I′), are nonribosomal polypeptides that belong to a family of depsipeptides produced by the myxobacterium Chondromyces crocatus NPH-MB180. These depsipeptides have been shown to be highly potent and selective human kallikrein 7 (hK7) and elastase inhibitors. Human kallikrein 7 is an enzyme with serine protease activity and is a potential target for the treatment of atopic dermatitis. Detailed physico-chemical data of the novel compounds, as well as fermentation and extraction methods, have been described in PCT patent application PCT/EP08/060,689, published as WO2009/024527.

As used herein, the term “compound of formula (I′)” or “depsipeptides of formula (I′)” will refer to the compounds of formula (I′) as defined above, and in particular to the non ribosomal peptides of formula (II), (III), (IV), (V), (VI), (VII), (XI), (XII), (XIII), (XIV) and/or (XVIII) as described in FIG. 1, and any derivatives retaining substantially the same protease activity. Examples of such derivatives are further described in PCT patent application published as WO2009/024527.

As used herein, the term “compound of formula (I′)” or “depsipeptides of formula (I′)” will refer to the compounds of formula (I′) as defined above, and in particular to the non ribosomal peptides of formula (II), (III), (IV), (V), (VI), (VII), (XI), (XII), (XIII), (XIV), (XVII) and/or (XVIII) as described in FIG. 1, and any derivatives retaining substantially the same protease activity.

The technical problem underlying the present invention is the provision of the biosynthetic cluster or functional parts thereof, involved in the biosynthesis of the depsipeptides of formula (I) or (I′).

The technical problem is solved by provision of the embodiments characterized in the claims.

Another technical problem underlying the present invention is the provision of repressible promoters appropriate for heterologous gene expression, for example for the synthesis of a recombinant protein of interest.

The present invention relates in a first embodiment to the provision of (1) a polynucleotide comprising one or more functional fragments of a biosynthetic gene cluster encoding a non ribosomal peptide synthase (NRPS), designated hereafter NRPS2 and involved in the production of a compound of formula (I) or (I′) comprising:
(i) a nucleotide sequence that has at least 80%, particularly at least 85%, particularly at least 90%, particularly at least 95%, particularly at least 98% sequence identity to a sequence selected among the group consisting of SEQ ID NO: 1, 3, 5, 7, 9, 11, 13, 46, 48, 50, 52, 54, 56, 58 and 60 encoding a NRPS2 domain and/or the complement thereof;
(ii) a nucleotide sequence which hybridizes to the complementary strand of a nucleotide sequence selected among the group consisting of SEQ ID NO: 1, 3, 5, 7, 9, 11, 13, 46, 48, 50, 52, 54, 56, 58 or 60 encoding a NRPS2 domain and/or the complement thereof;
(iii) a nucleotide sequence encoding an amino acid sequence that has at least 60%, particularly at least 70%, particularly at least 80%, particularly at least 90%, particularly at least 95% sequence identity to a sequence selected among the group consisting of SEQ ID NO: 2, 4, 6, 8, 10, 12, 14, 47, 49, 51, 53, 55, 57, 59 or 61 representing a NRPS2 domain and/or the complement thereof;
(iv) a nucleotide sequence which hybridizes to the complementary strand of a nucleotide sequence encoding an amino acid selected among the group consisting of SEQ ID NO: 2, 4, 6, 8, 10, 12, 14, 47, 49, 51, 53, 55, 57, 59 or 61 representing a NRPS2 domain and/or the complement thereof;
(v) a nucleotide sequence that has at least 80%, particularly at least 85%, particularly at least 90%, particularly at least 95%, particularly at least 98% sequence identity to a sequence selected among the group consisting of SEQ ID NO: 15, SEQ ID NO:28 and/or the complement thereof; or
(vi) a nucleotide sequence which hybridizes to the complementary strand of a nucleotide sequence as depicted selected among the group consisting of SEQ ID NO: 15, SEQ ID NO:28 and/or the complement thereof;
wherein said nucleotide sequences according to (i) to (vi) encode an expression product which retains the activity of the corresponding NRPS domain(s) represented by the reference sequence(s) of SEQ ID NOs: 2, 4, 6, 8, 10, 12, 14, 47, 49, 51, 53, 55, 59 and/or 61.

In a second embodiment, (2) a polynucleotide according to embodiment (1) is provided, wherein said polynucleotide encodes an expression product which retains the activity of one or more of the following NRPS2 domains:
(i) the thiolation domain of SEQ ID NO:47;
(ii) the condensation domain of SEQ ID NO:49;
(iii) the adenylation domain for Proline of SEQ ID NO:51;
(iv) the thiolation domain of SEQ ID NO:53;
(v) the condensation domain of SEQ ID NO:2
(vi) the adenylation domain for isoleucine of SEQ ID NO:4;
(vii) the thiolation domain of SEQ ID NO:6;
(viii) the condensation domain of SEQ ID NO:8
(ix) the adenylation domain for tyrosine of SEQ ID NO:10;
(x) the N-methylation domain of SEQ ID NO:12;
(xi) the thyolation domain of SEQ ID NO:14;
(xii) the condensation domain of SEQ ID:55;
(xiii) the adenylation domain for isoleucine of SEQ ID NO:57;
(xiv) the thiolation domain of SEQ ID NO:59; and/or,
(xv) the thioesterase domain of SEQ ID NO61.

In a specific embodiment of embodiment (2), said polynucleotides encodes a NRPS2 for producing a compound of formula (I) or (I′) comprising a nucleotide sequence encoding an amino acid sequence as depicted in SEQ ID NO:29.

In a third embodiment, (3) the present invention relates to a polynucleotide comprising one or more functional fragments of a biosynthetic gene cluster encoding NRPS1, a NRPS involved in the production of a compound of formula (I) or (I′) comprising:
(i) a nucleotide sequence that has at least 80%, particularly at least 85%, particularly at least 90%, particularly at least 95%, particularly at least 98% sequence identity to a sequence selected among the group consisting of SEQ ID NO: 30, 32, 34, 36, 38, 40, 42 and 44 encoding a NRPS domain and/or the complement thereof;
(ii) a nucleotide sequence which hybridizes to the complementary strand of a nucleotide sequence selected among the group consisting of SEQ ID NO: 30, 32, 34, 36, 38, 40, 42 and 44 encoding a NRPS domain and/or the complement thereof;
(iii) a nucleotide sequence encoding an amino acid sequence that has at least 60%, particularly at least 70%, particularly at least 80%, particularly at least 90%, particularly at least 95% sequence identity to a sequence selected among the group consisting of SEQ ID NO: 31, 33, 35, 37, 39, 41, 43, 45 representing a NRPS1 domain and/or the complement thereof;
(iv) a nucleotide sequence which hybridizes to the complementary strand of a nucleotide sequence encoding an amino acid selected among the group consisting of SEQ ID NO: 31, 33, 35, 37, 39, 41, 43, 45 representing a NRPS1 domain and/or the complement thereof;
(v) a nucleotide sequence that has at least 80%, particularly at least 85%, particularly at least 90%, particularly at least 95%, particularly at least 98% sequence identity to a sequence selected among the group consisting of SEQ ID NO: 26 and/or the complement thereof; or
(vi) a nucleotide sequence which hybridizes to the complementary strand of a nucleotide sequence as depicted selected among the group consisting of SEQ ID NO: 26 and/or the complement thereof;
(vii) wherein said nucleotide sequences according to (i) to (vi) still encode an expression product which retains the activity of the corresponding NRPS domain(s) represented by the reference sequences of SEQ ID NOs: SEQ ID NO: 31, 33, 35, 37, 39, 41, 43, 45.

In a fourth embodiment, a polynucleotide according to embodiment (3) encodes an expression product which retains the activity of the one or more of following NRPS1 domains:
(i) the loading domain of SEQ ID NO:31;
(ii) the adenylation domain for glutamine of SEQ ID NO:33;
(iii) the thiolation domain of SEQ ID NO:35;
(iv) the condensation domain of SEQ ID NO:37;
(v) the adenylation domain for threonine of SEQ ID NO:39;
(vi) the thiolation domain of SEQ ID NO:41;
(vii) the condensation domain of SEQ ID NO:43; and,
(viii) the adenylation domain for leucine of SEQ ID NO:45.

In a specific embodiment of embodiment (4), a polynucleotide encodes a NRPS1 for producing a compound of formula (I) or (I′) comprising a nucleotide sequence encoding an amino acid sequence as depicted in SEQ ID NO: 27.

In another embodiment, the invention relates to a polypeptide encoded by one or more polynucleotide described above. In particular, said polypeptide is appropriate for producing a compound of formula (I) or (I′) comprising an amino acid sequence selected among the group consisting of:
(i) SEQ ID NO:27 representing a NRPS1, SEQ ID NO:29 representing a second NRPS2, SEQ ID NO:63 representing a cytochrome P450; and,
(ii) a functional variant of an amino acid sequence listed in (i), having 60%, particularly at least 70%, particularly at least 80%, particularly at least 90%, particularly at least 95% sequence identity to the reference sequence listed in (i) and retaining substantially the same catalytic function.

The invention further relates to a polynucleotide comprising a nucleotide sequence encoding one or more of said polypeptides described above.

In still another embodiment, the invention provides a polynucleotide comprising
(i) a nucleotide sequence encoding SEQ ID NO:27 or a functional variant thereof; and
(ii) a nucleotide sequence encoding SEQ ID NO:29 or a functional variant thereof.

Such polynucleotide may further comprise a nucleotide sequence encoding SEQ ID NO:63 or a functional variant thereof. In one specific embodiment, said polynucleotide is isolated from Chondromyces crocatus strain NPH-MB180 having accession number DSM 19329.

The invention further provides an expression vector comprising a polynucleotide as defined in any of the preceding embodiments, wherein the open reading frames are operatively linked with transcriptional and translational sequences.

In a further embodiment, a host cell is provided, transfected with and expressing a polynucleotide or an expression vector as defined in any of the preceding embodiments, particularly, a host cell for the heterologous production of a compound of formula (I) or (I′) or a compound of formula (II) to (VII), (XI) to (XIV) and (XVII) and (XVIII).

In another embodiment, the invention relates to a method of preparing a compound of formula (I) or (I′) or of formula (II) to (VII), (XI) to (XIV) and (XVII) and (XVIII), comprising culturing a host cell as described in the preceding embodiment under conditions such that said compound is produced.

In one embodiment, the invention relates to an antibody that specifically binds to the polypeptide or to the NRPS or NRPS domains according to any of the preceding embodiments and to the use of said antibody, i.e., for purification of the polypeptide or NRPS.

In one embodiment, a pharmaceutical composition is provided comprising the polynucleotide, the vector, the polypeptide, the NRPS or NRPS domains or the antibody as defined in any of the preceding embodiments.

In one embodiment, a pharmaceutical composition is provided comprising the depsipeptides of formula (I) or (I′) obtainable or as obtained by culturing a recombinant host cell containing the polynucleotides of the invention under suitable as defined in any of the preceding embodiments.

In one embodiment, the invention relates to said depsipeptides of formula (I) or (I′) for the preparation of a pharmaceutical composition for use in treating and/or diagnosis of a disease or condition, i.e., atopic dermatitis. In one particular embodiment, the depsipeptides of formula (I) or (I′) are a selective human kallikrein (hK7) and elastase inhibitors, particularly an inhibitor of a selective human kallikrein (hK7), which has an enzyme activity, particularly a serine protease activity.

In a further embodiment of the invention, a biosynthetic gene cluster is provided encoding a NRPS involved in the production of a compound of formula (I) or (I′) comprising a polynucleotide as defined in any of the preceding embodiments.

In another embodiment of the invention, a polynucleotide sequence as defined in any of the preceding embodiments is provided for the identification of the biosynthetic gene cluster according to the invention obtainable by a method, comprising the (a) constructing of a nucleotide library composed of the genomic DNA of Chondromyces crocatus strain or related strain; (b) cultivation of the library strains as colonies; and (c) analyzing the grown colonies with a probe molecule based on a polynucleotide as defined in any of the preceding embodiments for the identification of clones containing the NRPS gene cluster, and (d) identifying the NRPS gene cluster.

The gist of the present invention lies in the provision of a biosynthetic cluster or functional parts thereof, involved in the biosynthesis of depsipeptides of formula (I) or (I′), particularly of the depsipeptides of formula (II) to (VII), (XI) to (XIV) and (XVII) and (XVIII). It is particularly advantageous that the identification of a biosynthetic cluster for a depsipeptide of formula (I) or (I′) can be used for the heterologous expression of said depsipeptide(s).

“Nonribosomal peptides” are meant to refer to a class of peptides belonging to a family of complex natural products built from simple amino acid monomers. They are synthesized in many bacteria and fungi by large multifunctional proteins called nonribosomal peptide synthetases (NRPS). A unique feature of NRPS system is the ability to synthesize peptides containing proteinogenic as well as non-proteinogenic amino acids.

A “Nonribosomal Peptide Synthase” (NRPS) is meant to refer to a large multifunctional protein which is organized into coordinated groups of active sites termed modules, in which each module is required for catalyzing one single cycle of product length elongation and modification of that functional group. The number and order of module and the type of domains present within a module on each NRPS determines the structural variation of the resulting peptide product by dictating the number, order, choice of the amino acid to be incorporated and the modification associated with a particular type of elongation.

The term “domain” refers to a functional part of a protein essential for a catalytic activity. Such domains are conserved among enzymes from different species carrying the same catalytic activity

The minimum set of domains required for an elongation cycle consist of a module with Adenylation (A), Thiolation (T) or Peptidyl Carrier Protein (PCP), and Condensation (C) domain.

The “Adenylation domain” is responsible for substrate selection and its covalent fixation on the phospho-pantethein arm of T domain as thioester, through AMP-derivative intermediate.

The C domain catalyzes the formation of peptide bond between an aminoacyl- or peptidyl-S-PCP from the upstream module and the aminoacyl moeity attached to the PCP in the corresponding downstream module. The result is peptide elongation by one residue fixed to the PCP domain in the downstream module. Optional modifying domain could be present for substrate epimerization, N-methylation and heterocyclization. The modules could remain on a single or multiple polypeptide chains.

In most cases, there is an extreme C-terminal Thioesterase (TE) domain in the last module responsible for the release/cyclization of the final product.

### 1. Polynucleotides Encoding the Biosynthetic Gene Clusters for Producing a Compound of Formula (I) or (I′)

The following table 1 describes specific examples of polynucleotides of the biosynthetic gene clusters for a compound of formula (I) or (I′) and their respective function and amino acid sequence.

TABLE 1
Depsipeptide biosynthetic gene cluster open
reading frames and functional domains.

| | | | | Nucle- | Pro- |
|---|---|---|---|---|---|
| | Do- | | | otide | tein |
| ORF | main | Coordinates¹ | Function | SEQ ID | SEQ ID |
| 1 | | 7537-9100 | Uncharacterized secreted | 16 | 17 |
| | | | protein | | |
| 2 | | 9120-10247 | Uncharacterized protein | 18 | 19 |
| 3 | | 10284-13094 | Putative Protease | 20 | 21 |
| 4 | | 13437-15095 | Permease 1 | 22 | 23 |
| 5 | | 15127-16806 | Permease 2 | 24 | 25 |
| 6 | | 16964-26041 | Nonribosomal peptide | 26 | 27 |
| | | | synthetase 1 (NRPS 1) | | |
| | 6.1 | 17123-18439 | Loading domain | 30 | 31 |
| | | | (Condensation domain) | | |
| | 6.2 | 18455-20008 | Adenylation domain | 32 | 33 |
| | | | (Gln) | | |
| | 6.3 | 20039-20233 | Thiolation domain | 34 | 35 |
| | 6.4 | 20294-21577 | Condensation domain | 36 | 37 |
| | 6.5 | 21593-23197 | Adenylation domain | 38 | 39 |
| | | | (Thr) | | |
| | 6.6 | 23228-23422 | Thiolation domain | 40 | 41 |
| | 6.7 | 23498-24781 | Condensation domain | 42 | 43 |
| | 6.8 | 24797-26041 | Adenylation domain | 44 | 45 |
| | | | (Leu) | | |
| 7 | | 26138-41365 | Nonribosomal peptide | 28 | 29 |
| | | | synthetase 2 (NRPS 2) | | |
| | 7.1 | 26380-26574 | Thiolation domain | 46 | 47 |
| | 7.2 | 26663-27946 | Condensation domain | 48 | 49 |
| | 7.3 | 27983-29572 | Adenylation domain | 50 | 51 |
| | | | (Pro) | | |
| | 7.4 | 29597-29791 | Thiolation domain | 52 | 53 |
| | 7.5 | 29837-31165 | Condensation domain | 1 | 2 |
| | 7.6 | 31170-32596 | Adenylation domain | 3 | 4 |
| | | | (Ile) | | |
| | 7.7 | 32759-32953 | Thiolation domain | 5 | 6 |
| | 7.8 | 33005-34330 | Condensation domain | 7 | 8 |
| | 7.9 | 34352-35908 | Adenylation domain | 9 | 10 |
| | | | (Tyr) | | |
| | 7.10 | 35741-36970 | N-methylation domain | 11 | 12 |
| | 7.11 | 37166-37360 | Thiolation domain | 13 | 14 |
| | 7.12 | 37406-38734 | Condensation domain | 54 | 55 |
| | 7.13 | 38738-40306 | Adenylation domain | 56 | 57 |
| | | | (Ile) | | |
| | 7.14 | 40328-40522 | Thiolation domain | 58 | 59 |
| | 7.15 | 40586-41317 | Thioesterase domain | 60 | 61 |
| 8 | | 41460-43295 | Cytochrome P450 | 62 | 63 |

¹Coordinates in nucleotides of Biosynthetic Gene Cluster Containing Scaffold.

The isolated biosynthetic gene cluster for the synthesis of the depsipeptides of formula (I) or (I′) is composed of 8 Open Reading Frames (ORFs), including ORF6 and ORF7 coding for non-ribosomal peptide synthetase, also referred as NRPS1 and NRPS2. NRPS1 and NRPS2 contains NRPS domains and corresponding presumed function is listed in Table 1.

The meaning of the terms “polynucleotide(s)”, “polynucleotide sequence” and “polypeptide” is well known in the art, and the terms are, if not otherwise defined herein, used accordingly in the context of the present invention (e.g. Seq ID NOs 1, 3, 5, 7, 9, 11, 13, 15, 16, 18, 20, 22, 24, 26, 28, 30, 32, 34, 36, 38, 40, 42, 44, 46, 48, 50, 52, 54, 56, 58, 60, 62, respectively). For example, “polynucleotide sequence” as used herein refers to all forms of naturally occurring or recombinantly generated types of nucleic acids and/or nucleotide sequences as well as to chemically synthesized nucleic acids/nucleotide sequences. This term also encompasses nucleic acid analogs and nucleic acid derivatives such as, e.g., locked DNA, PNA, oligonucleotide thiophosphates and substituted ribo-oligonucleotides. Furthermore, the term “polynucleotide sequence” also refers to any molecule that comprises nucleotides or nucleotide analogs.

Preferably, the term “polynucleotide sequence” refers to a nucleic acid molecule, i.e. deoxyribonucleic acid (DNA) and/or ribonucleic acid (RNA). The “polynucleotide sequence” in the context of the present invention may be made by synthetic chemical methodology known to one of ordinary skill in the art, or by the use of recombinant technology, or may be isolated from natural sources, or by a combination thereof. The DNA and RNA may optionally comprise unnatural nucleotides and may be single or double stranded. “Polynucleotide sequence” also refers to sense and anti-sense DNA and RNA, that is, a polynucleotide sequence which is complementary to a specific sequence of nucleotides in DNA and/or RNA.

Furthermore, the term “polynucleotide sequence” may refer to DNA or RNA or hybrids thereof or any modification thereof that is known in the state of the art (see, e.g., U.S. Pat. No. 5,525,711, U.S. Pat. No. 4,711,955, U.S. Pat. No. 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mRNA, antisense RNA, ribozymal or a DNA encoding such RNAs or chimeroplasts (Gamper, Nucleic Acids Research, 2000, 28, 4332-4339). Said polynucleotide sequence may be in the form of a plasmid or of viral DNA or RNA. “Polynucleotide sequence” may also refer to (an) oligonucleotide(s), wherein any of the state of the art modifications such as phosphothioates or peptide nucleic acids (PNA) are included.

The terms “gene cluster” or “biosynthetic gene cluster” refer to a group of genes or variants thereof involved in the biosynthesis of the depsipeptides of Formula (I) or (I′). Genetic modification of gene cluster or biosynthetic gene cluster refer to any genetic recombinant techniques known in the art including mutagenesis, inactivation, or replacement of nucleic acids that can be applied to generate variants of the compounds of Formula (I) or (I′). Genetic modification of gene cluster or biosynthetic gene cluster refers to any genetic recombinant techniques known in the art including mutagenesis, inactivation, or replacement of nucleic acids that can be applied to generate genetic variants of compounds of Formula (I) or (I′).

A DNA or nucleotide “coding sequence” or “sequence encoding” a particular polypeptide or protein, is a DNA sequence which is transcribed and translated into a polypeptide or protein when placed under the control of appropriate regulatory sequences.

In a particular embodiment the polynucleotides of the present invention (e.g. Seq ID NOs 1, 3, 5, 7, 9, 11, 13, 15, 16, 18, 20, 22, 24, 26, 28, 30, 32, 34, 36, 38, 40, 42, 44, 46, 48, 50, 52, 54, 56, 58, 60, 62, respectively) can be used in combination. Alternatively, the invention relates to fragment or functional variant of Seq ID NOs 1, 3, 5, 7, 9, 11, 13, 15, 16, 18, 20, 22, 24, 26, 28, 30, 32, 34, 36, 38, 40, 42, 44, 46, 48, 50, 52, 54, 56, 58, 60, 62.

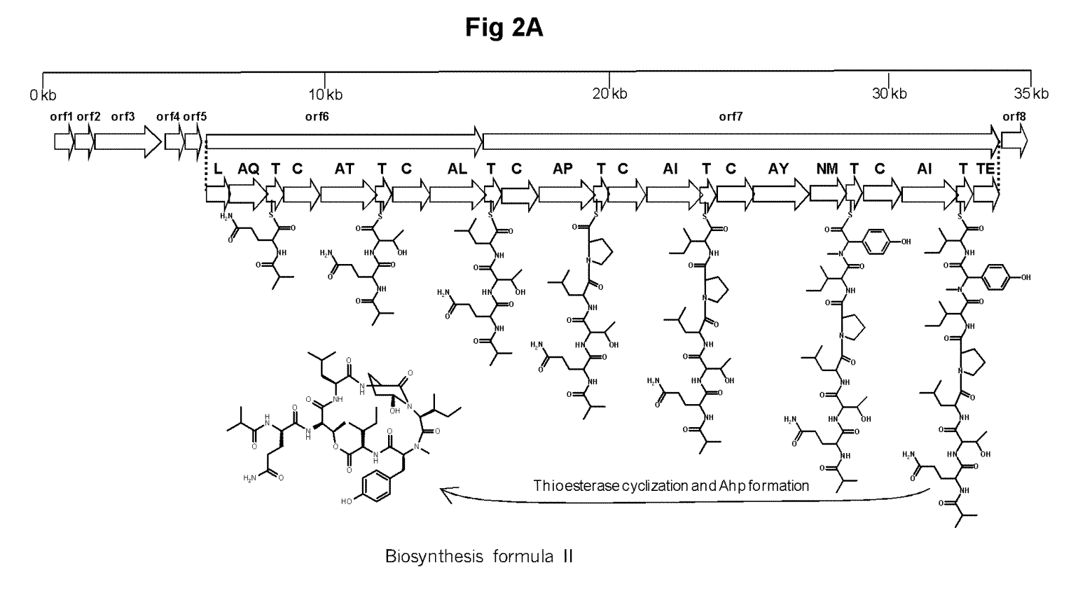

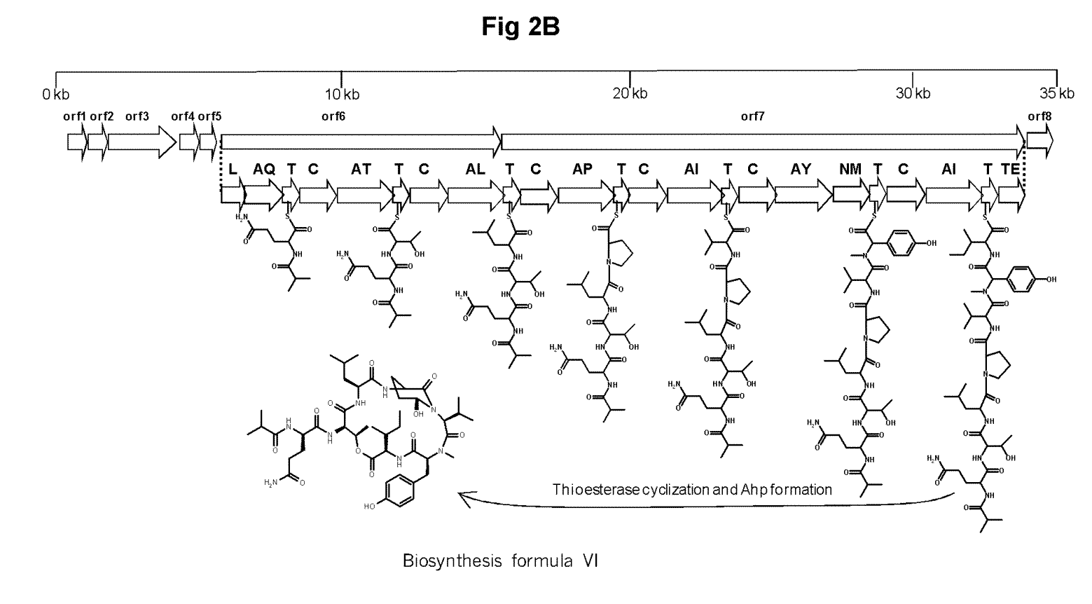

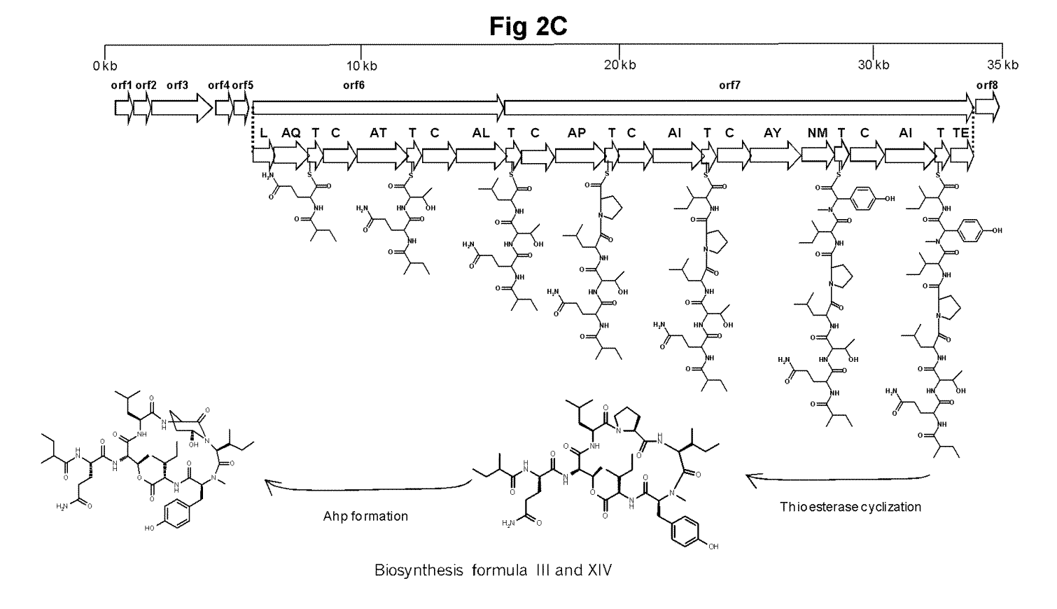

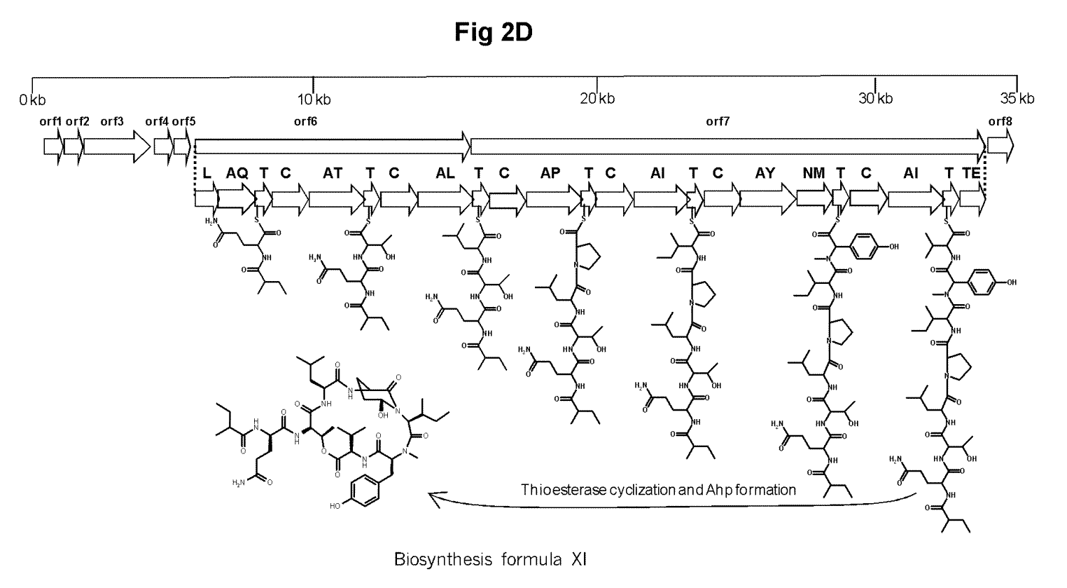

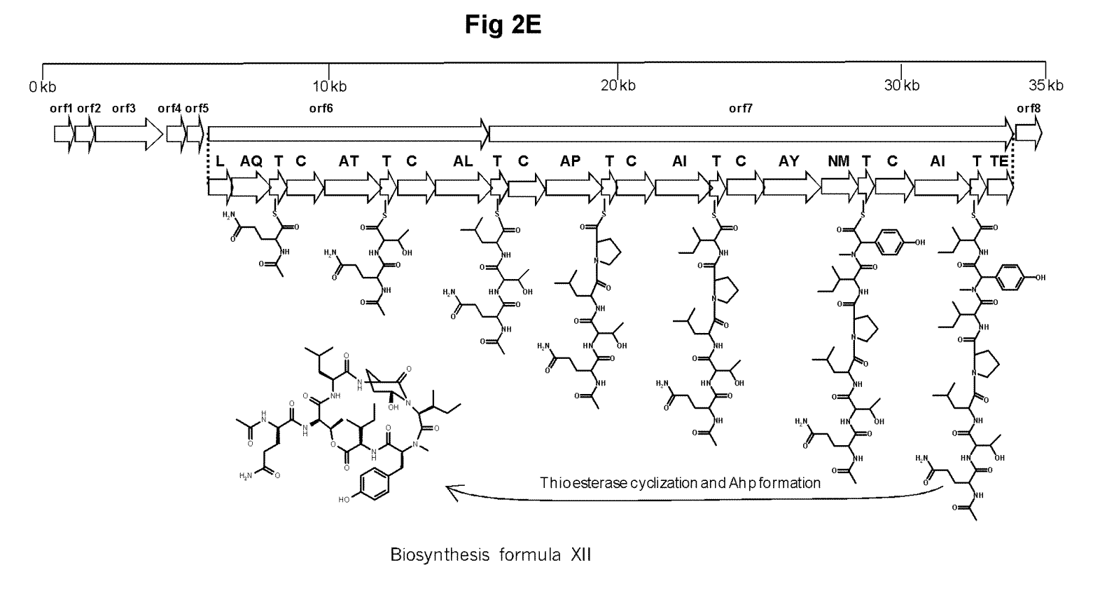

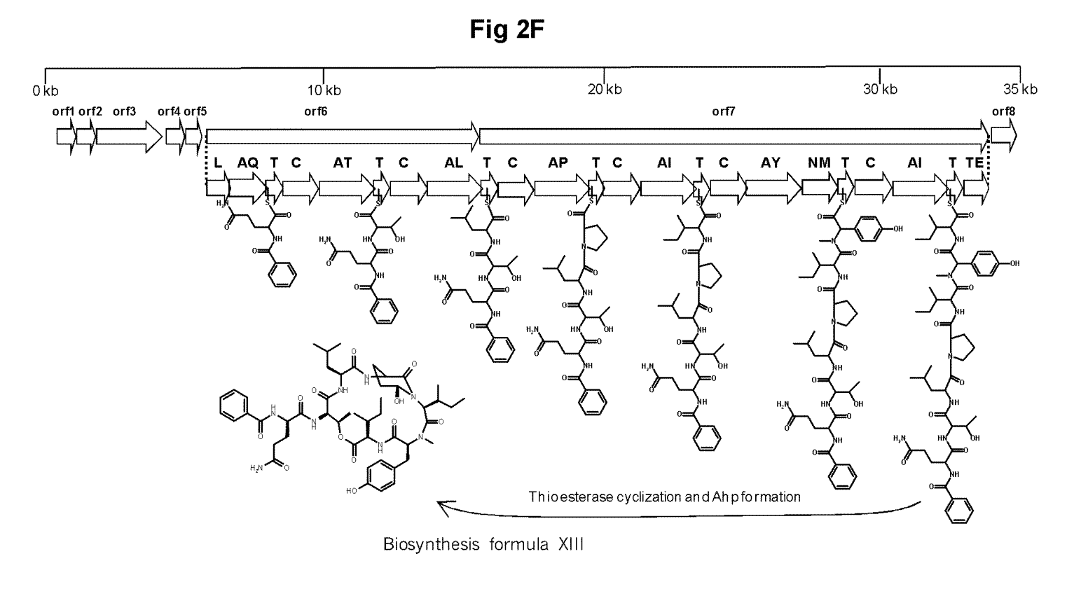

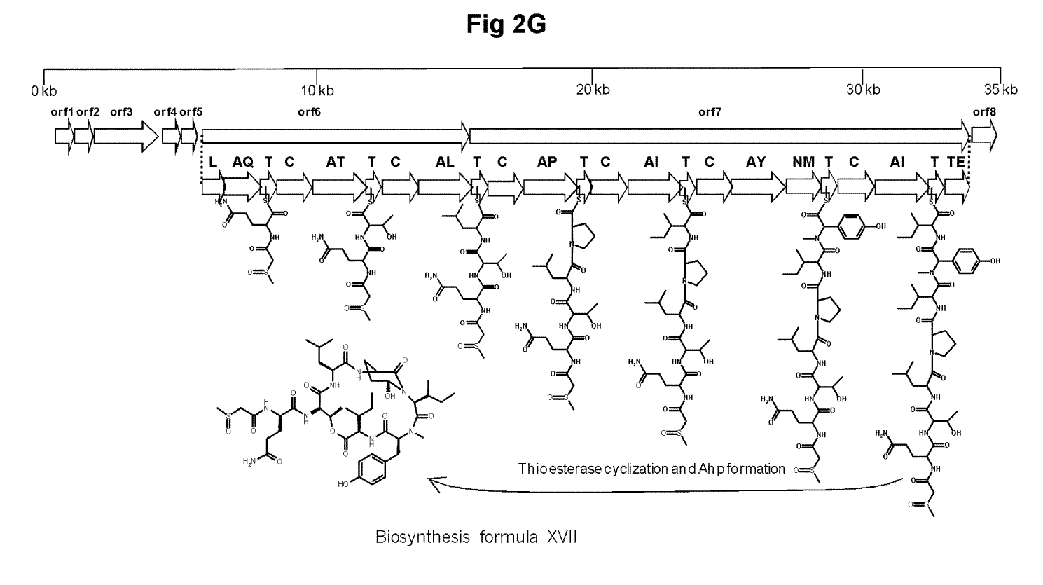

In context of polynucleotide sequences the term “fragment thereof” or “functional fragment thereof” refers in particular to (a) fragment(s) or a mutant variant of nucleic acid molecules. A “fragment of a polynucleotide” may, for example, encode a polypeptide of the present invention (e.g. a polypeptide as shown in SEQ ID NOs 2, 4, 6, 8, 10, 12 or 14) having at least one amino acid deletion whereby said polypeptide substantially retains the same function as the wild type polypeptide (the function of each polypeptide is described in Table 1 and FIG. 2 in more detail). Such a shortened polypeptide may be considered as a functional fragment of a polypeptide of the present invention (e.g. as shown in SEQ ID NOs 2, 4, 6, 8, 10, 12 or 14, 17, 19, 21, 23, 25, 27, 29, 31, 33, 35, 37, 39, 41, 43, 45, 47, 49, 51, 53, 55, 57, 59, 61, 63).

A “functional variant of a polynucleotide” may, for example, encode a polypeptide of the present invention (e.g. a polypeptide as shown in SEQ ID NOs 2, 4, 6, 8, 10, 12 or 14, 17, 19, 21, 23, 25, 27, 29, 31, 33, 35, 37, 39, 41, 43, 45, 47, 49, 51, 53, 55, 57, 59, 61, 63) having at least one amino acid substitution or addition whereby said polypeptide preferably retains the same function as the wild type polypeptide (the function of each polypeptide is described in Table 1 and FIG. 2 in more detail). Such a shortened polypeptide may be considered as a functional fragment of a polypeptide of the present invention (e.g. as shown in SEQ ID NOs 2, 4, 6, 8, 10, 12 or 14, 17, 19, 21, 23, 25, 27, 29, 31, 33, 35, 37, 39, 41, 43, 45, 47, 49, 51, 53, 55, 57, 59, 61, 63).

The functional variants of a polynucleotide/polypeptide of the invention have a sequence identity, of at least 50%, 55%, 60%, preferably of at least 70%, more preferably of at least 80%, 85%, 90%, 95% and even most preferably of at least 99% to their corresponding original polynucleotide/polypeptide sequences as described in Table 1. For example, a polypeptide has at least 50%, 55% 60% preferably at least 70%, more preferably at least 80%, 85%, 90%, 95% and most preferably at least 99% identity/homology to the polypeptide shown in SEQ ID NO 2, 4, 6, 8, 10, 12 or 14, 17, 19, 21, 23, 25, 27, 29, 31, 33, 35, 37, 39, 41, 43, 45, 47, 49, 51, 53, 55, 57, 59, 61, 63 respectively.

With respect to a nucleotide sequence of a non-ribosomal peptide synthases (NRPS) or other ORFs described in Table 1, the term “fragment” as used herein means a nucleotide sequence being at least 7, at least 10, at least 15, at least 20, at least 30, at least 50, at least 100, at least 150, at least 200, at least 250, at least 300, at least 350, at least 400, at least 450, at least 500, at least 550, at least 600, at least 650 or at least 700 nucleotides in length.

The term “hybridizes” used herein refers to hybridization under conventional hybridization conditions, preferably under stringent conditions, as for instance described in Sambrook and Russell (2001), Molecular Cloning: A Laboratory Manual, CSH Press, Cold Spring Harbor, N.Y., USA. If not further specified, the conditions are preferably non-stringent. Said hybridization conditions may be established according to conventional protocols described, e.g., in Sambrook (2001) loc. cit. The setting of conditions is well within the skill of the artisan and can be determined according to protocols described in the art. Thus, the detection of only specifically hybridizing sequences will usually require stringent hybridization and washing conditions. As a non-limiting example, highly stringent hybridization may occur under the following conditions:
Hybridization buffer:
   2×SSC; 10×Denhardt solution (Fikoll 400+PEG+BSA;
   ratio 1:1:1); 0.1% SDS; 5 mM EDTA; 50 mM Na₂HPO₄;
   250 μg/ml of herring sperm DNA; 50 μg/ml of tRNA; or
   0.25 M of sodium phosphate buffer, pH 7.2;
   1 mM EDTA
   7% SDS
Hybridization temperature T=60° C.
Washing buffer: 2×SSC; 0.1% SDS
Washing temperature T=60° C.

Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

Polynucleotide sequences which are capable of hybridizing with the polynucleotide sequences provided herein are also part of the invention and can for instance be isolated from genomic libraries or cDNA libraries of animals or from DNA libraries of microbes. Preferably, such polynucleotides are of microbial origin, particularly of microbes belonging to the class of proteobacteria, particularly Deltaproteobacteria, particularly Myxococcales, particularly Sorangiineae, particularly Polyangiaceae, but especially Chondromyces, such as Chondromyces crocatus or an improved strain thereof.

Alternatively, such variant nucleotide sequences according to the invention can be prepared by genetic engineering or chemical synthesis. Such polynucleotide sequences being capable of hybridizing may be identified and isolated by using the polynucleotide sequences described herein or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Nucleotide sequences comprising the same or substantially the same nucleotide sequences as indicated in the listed SEQ ID NOs, or parts/fragments thereof, can, for instance, be used as hybridization probes. A fragment can also be useful as a probe or a primer for diagnosis, sequencing or cloning of the NRPS gene cluster. The fragments used as hybridization probes can also be synthetic fragments which are prepared by usual synthesis techniques, the sequence of which is substantially identical with that of a nucleotide sequence according to the invention.

As used herein, the percent identity between the two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

Preferably, the degree of identity/homology is determined by comparing the respective sequence with the nucleotide sequences as indicated in the listed SEQ ID NOs. When the sequences which are compared do not have the same length, the degree of homology preferably refers to the percentage of nucleotide residues in the shorter sequence which are identical to nucleotide residues in the longer sequence. The degree of homology can be determined conventionally using known computer programs such as the DNASTAR program with the ClustalW analysis. This program can be obtained from DNASTAR, Inc., 1228 South Park Street, Madison, Wis. 53715 or from DNASTAR, Ltd., Abacus House, West Ealing, London W13 OAS UK (support@dnastar.com) and is accessible at the server of the EMBL outstation.

When using the Clustal analysis method to determine whether a particular sequence is, for instance, 80% identical to a reference sequence the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

If the two nucleotide sequences to be compared by sequence comparisons differ in identity refers to the shorter sequence and that part of the longer sequence that matches the shorter sequence. In other words, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence.

In general, the person skilled in the art knows how nucleic acid molecules can be obtained, for instance, from natural sources or may also be produced synthetically or by recombinant techniques, such as PCR These nucleic acid molecules and include modified or derivatized, nucleic acid molecules as can be obtained by applying techniques described in the pertinent literature.

Identity, moreover, means that there is a functional and/or structural equivalence between the corresponding nucleotide sequence or polypeptides, respectively (e.g. polypeptides encoded thereby). Nucleotide/amino acid sequences which have at least 50%, 55%, 60%, preferably of at least 70%, more preferably of at least 80%, 85% 90%, 95% and even most preferably of at least 99% identity to the herein-described particular nucleotide/amino acid sequences may represent derivatives/variants of these sequences which, preferably, have the same biological function. They may be either naturally occurring variations, for instance sequences from other ecotypes, varieties, species, etc., or mutations, and said mutations may have formed naturally or may have been produced by deliberate mutagenesis. Furthermore, the variations may be synthetically produced sequences. The allelic variants may be naturally occurring variants or synthetically produced variants or variants produced by recombinant DNA techniques. Deviations from the above-described polynucleotides may have been produced, e.g., by deletion, substitution, addition, insertion and/or recombination. The term “addition” refers to adding at least one nucleic acid residue/amino acid to the end of the given sequence, whereas “insertion” refers to inserting at least one nucleic acid residue/amino acid within a given sequence.

The variant polypeptides and, in particular, the polypeptides encoded by the different variants of the nucleotide sequences of the invention preferably exhibit certain characteristics they have in common. These include, for instance, biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

In one particular embodiment, the invention provides a polynucleotide which encodes one or more expression products which retains the activity of the one or more of following NRPS1 domains:
(i) the loading domain of SEQ ID NO:31;
(ii) the adenylation domain for glutamine of SEQ ID NO:33;
(iii) the thiolation domain of SEQ ID NO:35;
(iv) the condensation domain of SEQ ID NO:37;
(v) the adenylation domain for threonine of SEQ ID NO:39;
(vi) the thiolation domain of SEQ ID NO:41;
(vii) the condensation domain of SEQ ID NO:43; and,
(viii) the adenylation domain for leucine of SEQ ID NO:45.

In a specific embodiment, the polynucleotide encodes one or more expression products which retain the activity of all the NRPS1 domains described above.

In an alternative embodiment, the polynucleotide encodes one or more expression products which retain the activity of all the NRPS1 domains described, except that one, two or three adenylation domains are substituted for one or more adenylation domains with different amino acid specificity.

In another specific embodiment, the invention provides a polynucleotide which encodes one or more expression products which retains the activity of the one or more of following NRPS2 domains:
(i) the thiolation domain of SEQ ID NO:47;
(ii) the condensation domain of SEQ ID NO:49;
(iii) the adenylation domain for Proline of SEQ ID NO:51;
(iv) the thiolation domain of SEQ ID NO:53;
(v) the condensation domain of SEQ ID NO:2
(vi) the adenylation domain for isoleucine of SEQ ID NO:4;
(vii) the thiolation domain of SEQ ID NO:6;
(viii) the condensation domain of SEQ ID NO:8
(ix) the adenylation domain for tyrosine of SEQ ID NO:10;
(x) the N-methylation domain of SEQ ID NO:12;
(xi) the thyolation domain of SEQ ID NO:14;
(xii) the condensation domain of SEQ ID:55;
(xiii) the adenylation domain for isoleucine of SEQ ID NO:57;
(xiv) the thiolation domain of SEQ ID NO:59; and,
(xv) the thioesterase domain of SEQ ID NO61.

In a specific embodiment, the polynucleotide encodes one or more expression products which retain the activity of all the NRPS2 domains described above. In an alternative embodiment, the polynucleotide encodes one or more expression products which retain the activity of all the NRPS1 domains described, except that one, two, three or four adenylation domains are substituted for another adenylation domain with different amino acid specificity.

ORF6 encoding NRPS1, ORF7 encoding NRPS2 and ORF8 encoding cytochrome P450 are presumed to encode the core enzymes for the biosynthesis of the depsipeptides of formula (I) or (I′). Therefore, in a further aspect, the present invention relates to a polynucleotide comprising

(i) a nucleotide sequence encoding SEQ ID NO:27 (NRPS1) or a functional variant thereof; and,

(ii) a nucleotide sequence encoding SEQ ID NO:29 (NRPS2) or a functional variant thereof.

The polynucleotide may further comprise a nucleotide sequence encoding SEQ ID NO:63 or a functional variant thereof. In one specific embodiment, these polynucleotides are isolated from Chondromyces crocatus strain NPH-MB180 having accession number DSM19329.

### 2. The NRPS and Other Polypeptides Involved in the Production of a Compound of Formula (I) or (I′)

The invention further relates to the polypeptides encoded by the polynucleotides of the invention, in particular those described in Table 1, for example, NRPS1 and NRPS2. The invention further relates to their functional fragment and functional variant.

The present invention also relates to variants of the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 or fragments comprising at least 50, 75, 100, 150, 200, 300, 400 or 500 consecutive amino acids thereof. The term “variant” includes derivatives or analogs of these polypeptides. In particular, the variants may differ in amino acid sequence from the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 by 1, 2, 3, 4, 5 or more substitutions, additions, deletions, fusions and truncations, which may be present in any combination.

The variants may be naturally occurring or created in vitro. In particular, such variants may be created using genetic engineering techniques such as site directed mutagenesis, random chemical mutagenesis, exonuclease III deletion procedures, and standard cloning techniques. Alternatively, such variants, fragments, analogs, or derivatives may be created using chemical synthesis or modification procedures.

Other methods of making variants are also familiar to those skilled in the art. These include procedures in which nucleic acid sequences obtained from natural isolates are modified to generate nucleic acids that encode polypeptides having characteristics which enhance their value in industrial or laboratory applications. In such procedures, a large number of variant sequences having one or more nucleotide differences with respect to the sequence obtained from the natural isolate are generated and characterized. Preferably, these nucleotide differences result in amino acid changes with respect to the polypeptides encoded by the nucleic acids from the natural isolates.

For example, variants may be created using error prone PCR. In error prone PCR, DNA amplification is performed under conditions where the fidelity of the DNA polymerase is low, such that a high rate of point mutation is obtained along the entire length of the PCR product. Error prone PCR is described in Leung, D. W., et al., Technique, 1:11-15 (1989) and Caldwell, R. C. & Joyce G. F., PCR Methods Applic., 2:28-33 (1992). Variants may also be created using site directed mutagenesis to generate site-specific mutations in any cloned DNA segment of interest. Oligonucleotide mutagenesis is described in Reidhaar-Olson, J. F. & Sauer, R. T., et al., Science, 241:53-57 (1988). Variants may also be created using directed evolution strategies such as those described in U.S. Pat. Nos. 6,361,974 and 6,372,497. The variants of the polypeptides of SEQ ID NOS: 2, 4, 6, 8, 10, 12, 14 may be variants in which 1, 2, 3, 4, 5 or more of the amino acid residues of the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 are substituted with a conserved or non-conserved amino acid residue (preferably a conserved amino acid residue) and such substituted amino acid residue may or may not be one encoded by the genetic code.

Conservative substitutions are those that substitute a given amino acid in a polypeptide by another amino acid of like characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue.

Other variants are those in which one or more of the amino acid residues of the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 include a substituent group. Still other variants are those in which the polypeptide is associated with another compound, such as a compound to increase the half-life of the polypeptide (for example, polyethylene glycol). Additional variants are those in which additional amino acids are fused to the polypeptide, such as leader sequence, a secretory sequence, a proprotein sequence or a sequence that facilitates purification, enrichment, or stabilization of the polypeptide.

In some embodiments, the fragments, derivatives and analogs retain the same biological function or activity as the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63. The term “fragment thereof” as used herein in context of polypeptides, refers to a functional fragment which has essentially the same (biological) activity as the polypeptides defined herein (e.g. as shown in Seq ID NOs 2, 4, 6, 8, 10, 12 or 14, 17, 19, 21, 23, 25, 27, 29, 31, 33, 35, 37, 39, 41, 43, 45, 47, 49, 51, 53, 55, 57, 59, 61, 63 respectively) which may be) encoded by the polynucleotides of the present invention (e.g. Seq ID NOs 1, 3, 5, 7, 9, 11, 13, 15, 16, 18, 20, 22, 24, 26, 28, 30, 32, 34, 36, 38, 40, 42, 44, 46, 48, 50, 52, 54, 56, 58, 60, 62, respectively).

In other embodiments, the fragment, derivatives and analogs retain the same biological function or activity as the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63, except that at least one, two, three, four, five, six or seven adenylation domain is substituted by a different adenylation domain, thereby providing different amino acid specificity.

In other embodiments, the fragment, derivative or analogue includes a fused heterologous sequence that facilitates purification, enrichment, detection, stabilization or secretion of the polypeptide that can be enzymatically cleaved, in whole or in part, away from the fragment, derivative or analogue.

Another aspect of the present invention are polypeptides or fragments thereof which have at least 60%, at least 70%, at least 80%, at least 90%, or at least 95% identity to one of the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 or fragments comprising at least 50, 75, 100, 150, 200, 300, 400 or 500 consecutive amino acids thereof. It will be appreciated that amino acid “identity” includes conservative substitutions such as those described above.

The polypeptides or fragments having homology to one of the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 or fragments comprising at least 50, 75, 100, 150, 200, 300, 400 or 500 consecutive amino acids thereof may be obtained by isolating the nucleic acids encoding them using the techniques described above.

Alternatively, the homologous polypeptides or fragments may be obtained through biochemical enrichment or purification procedures. The sequence of potentially homologous polypeptides or fragments may be determined by proteolytic digestion, gel electrophoresis and/or microsequencing. The sequence of the prospective homologous polypeptide or fragment can be compared to one of the polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 or fragments comprising at least 50, 75, 100, 150, 200, 300, 400 or 500 consecutive amino acids thereof.

The polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 or fragments comprising at least 50, 75, 100, 150, 200, 300, 400 or 500 consecutive amino acids thereof comprising at least 40, 50, 75, 100, 150, 200 or 300 consecutive amino acids thereof may be used in a variety of applications. For example, the polypeptides or fragments, derivatives or analogs thereof may be used to catalyze biochemical reactions as described elsewhere in the specification.

The polypeptides of SEQ ID NOS: 2, 4, 6, 8, 10, 12, 14, 17, 19, 21, 23, 25, 27, 29, 31, 33, 35, 37, 39, 41, 43, 45, 47, 49, 51, 53, 55, 57, 59, 61, 63 or fragments comprising at least 50, 75, 100, 150, 200, 300, 400 or 500 consecutive amino acids thereof, may also be used to generate antibodies which bind specifically to the polypeptides or fragments, derivatives or analogues.

In a particular embodiment the polypeptides of the present invention (e.g. as shown in Seq ID NOs 2, 4, 6, 8, 10, 12 or 14, 17, 19, 21, 23, 25, 27, 29, 31, 33, 35, 37, 39, 41, 43, 45, 47, 49, 51, 53, 55, 57, 59, 61, 63 respectively) can be used in combination.

The term “activity” or “functionality” as used herein refers in particular to the capability of (a) polypeptide(s) or (a) fragment(s) thereof to elicit an enzymatic activity, e.g. peptide synthase activity for NRPS1 and NRPS2. A person skilled in the art will be aware that the (biological) activity of functionality as described herein often correlates with the expression level (e.g. protein/mRNA). If not mentioned otherwise, the term “expression” used herein refers to the expression of a nucleic acid molecule encoding a polypeptide/protein (or a fragment thereof) of the invention, whereas “activity” refers to activity of said polypeptide/protein. Methods/assays for determining the activity of polypeptides described herein are well known in the art.

### 3. Expression Vectors, Recombinant Host Cells and Methods of Preparing the Depsipeptides of Formula (I) or (I′)

The polynucleotides of the invention described herein are useful for example for heterologous expression of a compound of formula (I) or (I′). In specific embodiments, they are useful for heterologous expression of the compounds of formula (I′).

Accordingly, and in a further aspect, the present invention relates to a vector comprising the nucleic acid molecules described herein, more specifically expression vectors, and a recombinant host cell comprising the nucleic acid molecules and/or the vector.

The term “vector” as used herein particularly refers to plasmids, cosmids, bacterial artificial chromosomes (BAC), yeast artificial chromosomes, viruses, bacteriophages and other vectors commonly used in genetic engineering. In a preferred embodiment, the vectors of the invention are suitable for the transformation of cells, like fungal cells, cells of microorganisms such as yeast or bacterial cells or animal cells. An “expression vector” refers to a vehicle by which a nucleic acid can be introduced into a host cell, resulting in expression of the introduced sequence.

As discussed herein, polypeptides may be obtained by inserting a nucleic acid encoding the polypeptide into a vector such that the coding sequence is operatively linked to a sequence capable of driving the expression of the encoded polypeptide in a suitable host cell. For example, the expression vector may comprise a promoter, a ribosome binding site for translation initiation and a transcription terminator. The vector may also include appropriate sequences for modulating expression levels, an origin of replication and a selectable marker. Promoters suitable for expressing the polypeptide or fragment thereof in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Fungal promoters include the α factor promoter. Promoters suitable for expression in Pseudomonas putida includes, without limitation, the corresponding transcriptional promoters of the seven 16S rRNA genes present in the genome (PP 16SA, PP 16SB, PP 16SC, PP 16SD, PP 16SE, PP 16SF, PP 16SG), the transcriptional promoters of antibiotic resistance determinants, the transcriptional promoters of any ferric uptake repressor (Fur) regulated genes. A more detailed description of ferric uptage repressor (Fur) regulated promoters is provided further below. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

Mammalian expression vectors may also comprise an origin of replication, any necessary ribosome binding sites, a polyadenylation site, splice donors and acceptor sites, transcriptional termination sequences, and 5′ flanking nontranscribed sequences. In some embodiments, DNA sequences derived from the SV40 splice and polyadenylation sites may be used to provide the required nontranscribed genetic elements.

Vectors for expressing the polypeptide or fragment thereof in eukaryotic cells may also contain enhancers to increase expression levels. Enhancers are cis-acting elements of DNA, usually from about 10 to about 300 bp in length that act on a promoter to increase its transcription. Examples include the SV40 enhancer on the late side of the replication origin by 100 to 270, the cytomegalovirus early promoter enhancer, the polyoma enhancer on the late side of the replication origin, and the adenovirus enhancers.

In addition, the expression vectors preferably contain one or more selectable marker genes to permit selection of host cells containing the vector. Examples of selectable markers that may be used include genes encoding dihydrofolate reductase or genes conferring neomycin resistance for eukaryotic cell culture, genes conferring tetracycline or ampicillin resistance in E. coli, and the S. cerevisiae TRP1 gene. An example of suitable marker is the gentamicin resistance cassette aacCl. Other selectable markers could include nucleotide cassette that confers resistance to ampicilline (such as bla), chloramphenicol (such as cat), kanamycin (such as aacC2, aadB or other aminoglycoside modifying enzymes) or tetracycline (such as tetA or tetB).

The appropriate DNA sequence may be inserted into the vector by a variety of procedures. In general, the DNA sequence is ligated to the desired position in the vector following digestion of the insert and the vector with appropriate restriction endonucleases. Alternatively, appropriate restriction enzyme sites can be engineered into a DNA sequence by PCR. A variety of cloning techniques are disclosed in Ausbel et al. Current Protocols in Molecular Biology, John Wiley 503 Sons, Inc. 1997 and Sambrook et al., Molecular Cloning: A Laboratory Manual 2d Ed., Cold Spring Harbour Laboratory Press, 1989. Such procedures and others are deemed to be within the scope of those skilled in the art.

The vector may be, for example, in the form of a plasmid, a viral particle, or a phage. Other vectors include derivatives of chromosomal, nonchromosomal and synthetic DNA sequences, viruses, bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by Sambrook et al., Molecular Cloning: A Laboratory Manual, Second Edition, Cold Spring Harbor, N.Y., (1989).

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), pGEM1 (Promega Biotec, Madison, Wis., USA) pQE70, pQE60, pQE-9 (Qiagen), pD10, phiX174, pBluescript™ II KS, pNH8A, pNH16a, pNH18A, pNH46A (Stratagene), ptrc99a, pKK223-3, pKK233-3, pDR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and stable in the host cell.

The vector may be introduced into the host cells using any of a variety of techniques, including electroporation transformation, transfection, transduction, viral infection, gene guns, or Ti-mediated gene transfer. Where appropriate, the engineered host cells can be cultured in conventional nutrient media modified as appropriate for activating promoters, selecting transformants or amplifying the genes of the present invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

In a further aspect, the recombinant host cell of the present invention is capable of expressing or expresses the polypeptide encoded by the polynucleotide sequence of this invention. In a specific embodiment, the “polypeptide” comprised in the host cell may be a heterologous with respect to the origin of the host cell. An overview of examples of different expression systems to be used for generating the host cell of the present invention, for example the above-described particular one, is for instance contained in Glorioso et al. (1999), Expression of Recombinant Genes in Eukaryotic Systems, Academic Press Inc., Burlington, USA, Pauline Balbas and Argelia Lorence (2004), Recombinant Gene Expression: Reviews and Protocols, Second Edition: Reviews and Protocols (Methods in Molecular Biology), Humana Press, USA.

The transformation or genetically engineering of the host cell with a nucleotide sequence or the vector according to the invention can be carried out by standard methods, as for instance described in Sambrook and Russell (2001), Molecular Cloning: A Laboratory Manual, CSH Press, Cold Spring Harbor, N.Y., USA. Moreover, the host cell of the present invention is cultured in nutrient media meeting the requirements of the particular host cell used, in particular in respect of the pH value, temperature, salt concentration, aeration, antibiotics, vitamins, trace elements etc.

Generally, the host cell of the present invention may be a prokaryotic or eukaryotic cell comprising the nucleotide sequence, the vector and/or the polypeptide of the invention or a cell derived from such a cell and containing the nucleotide sequence, the vector and/or the polypeptide of the invention. In a preferred embodiment, the host cell comprises, for example due to genetic engineering, the nucleotide sequence or the vector of the invention in such a way that it contains the nucleotide sequences of the present invention integrated into the genome. Non-limiting examples of such a host cell of the invention (but also the host cell of the invention in general) may be a bacterial, yeast, fungus, plant, animal or human cell.

The term “host cell” or “isolated host cell” refer to a microorganism that carries genetic information necessary to produce compound of formula (I) or a compound of formula (I′), whether or not the organism is known to produce said compound. The term, as used herein, apply equally to organisms in which the genetic information to produce, e.g. the compound of formula (I) or (I′), is found in the organism as it exists in its natural environment, and to organisms in which the genetic information is introduced by recombinant techniques. The host cell may be any of the host cells familiar to those skilled in the art, including prokaryotic cells or eukaryotic cells. As representative examples of appropriate hosts, there may be mentioned: bacteria cells, such as E. coli, Streptomyces lividans, Streptomyces griseofuscus, Streptomyces ambofaciens, Bacillus subtilis, Salmonella typhimurium, Myxococcus xanthus, Sorangium cellulosum, Chondromyces crocatus and various species within the genera Pseudomonas, Streptomyces, Bacillus, and Staphylococcus, fungal cells, such as yeast, insect cells such as Drosophila S2 and Spodoptera Sf9, animal cells such as CHO, COS or Bowes melanoma, and adenoviruses. The selection of an appropriate host is within the abilities of those skilled in the art.

As source organisms contemplated herein are organisms included of Proteobacteria, preferably Deltaproteobacteria, more preferably Myxococcales, more preferably Sorangiineae, more preferably Polyangiaceae, most preferably Chondromyces of which Chondromyces crocatus or an improved strain thereof is most preferred.

The term “recombinant host cell”, as used herein, relates to a host cell, genetically engineered with the nucleotide sequence of the present invention or comprising the vector or the polypeptide or a fragment thereof of the present invention. The invention permits the production of depsipeptides of formula (I) or of formula (I′) to be expressed in a heterologous recombinant host cell, i.e., another strain than the natural producing strain. Although the examples illustrate use of a bacterial strain, any organism or expression system can be used as described herein. The choice of organism is dependent upon the needs of the skilled artisan. For example, a strain that is amenable to genetic manipulation may be used in order to facilitate modification and production of depsipeptides compounds.

In one specific embodiment, the host cell is selected among species of the genera Myxococcocus or Pseudomonas, for example, Pseudomonas putida. In one more specific embodiment, the recombinant host cells, e.g., Pseudomonas putida, comprises the nucleotides encoding NRPS1 (SEQ ID NO:27) and NRPS2 (SEQ ID NO:29) or functional variants thereof. It may further comprise the nucleotide sequence encoding cytochrome P450 of SEQ ID NO:63 or a functional variant. It may also comprise one or more of SEQ ID NO:17, SEQ ID NO:18, SEQ ID NO:21, SEQ ID NO:23, SEQ ID NO:25 and SEQ ID NO:27. Advantageously, each Open Reading Frame is under the control of functional transcriptional and translational sequences so that these ORFs are expressed under suitable conditions by the recombinant host cell. A specific example of heterologous expression in Pseudomonas putida is further described in the Examples below.

In accordance with the above, the invention relates in a further embodiment to a method for producing a compound of formula (I) or of formula (I′), comprising culturing the recombinant host cell under such conditions that the compound of formula (I) or formula (I′), for example, of formula (II) to (VII), (XI) to (XIV) and (XVII) and (XVIII) is synthesized, and recovering said compound.

The term “such conditions”, as used herein, refers to culture conditions of recombinant host cells in order to express and recover the compound of formula (I) or the compound of formula (I′). In one specific embodiment, the recombinant host cell is Pseudomonas putida. In another specific embodiment, the recombinant host cell is Pseudomonas putida and the cells are grown at a temperature of less than 30° C., for example, between 10 and 20° C., for example about 15° C.

In another specific embodiment, the growth medium contains isobutyric acid, for example between 1 and 5 g/l of isobutyric acid, for example about 2 g/l of isobutyric acid.

For example, the recombinant host cells of the invention may particularly be suitable for a potentiated or increased production of the depsipeptides of formula (I) or of formula (I′).

### 4. Use of Iron-Regulated Promoters in Heterologous Gene Expression

Another aspect of the invention relates to the heterologous gene expression or synthesis of recombinant proteins of interest in a host cell, for example in Pseudomonas host cells, such as Pseudomonas putida. In some instances, in particular where recombinant protein expression may impair growth of the bacteria, there is a need to control the heterologous gene expression so that it is inhibited until the transition stage of growth or until the host cell reach a healthy population density or most appropriate stage for heterologous gene expression. The inventors have shown that heterologous gene expression can be successfully regulated by Fur regulated promoters in a recombinant host cell, e.g., in Pseudomonas putida. Though the use of such promoters is described in the present application for heterologous expression of the biosynthetic gene cluster of depsipeptides, the Fur regulated promoters of the invention may have much wider use in the field for heterologous gene expression or synthesis of recombinant protein of interest.

The present invention therefore provides means for regulating and enhancing heterologous gene expression in a recombinant host cell, preferably a bacterial host cell, for example, in Pseudomonas species, such as Pseudomonas putida.

In one embodiment, the invention relates to an expression cassette for heterologous gene expression or for the synthesis of a recombinant protein of interest. Such expression cassette is a polynucleotide sequence that comprises at least the open reading frame encoding a mature recombinant protein of interest (hereafter referred as the coding sequence) operatively linked to an iron-regulated promoter.

As used herein within the context of “heterologous gene expression”, the term “recombinant protein of interest” refers to a protein that is not naturally expressed under the control of an iron-regulated promoter. In preferred embodiments, a recombinant protein of interest may be an enzyme, a therapeutic protein, including without limitation a hormone, a growth factor, an anti-coagulant, a receptor agonist or antagonist or decoy receptor), antibodies (including diagnostic or therapeutic) or alternative target-binding scaffolds such as, without limitation, fibronectin-derived proteins, single domain antibodies, single chain antibodies, nanobodies and the like.

As used herein in the context of an expression cassette, the term “operatively linked” refers to a polynucleotide sequence comprising a promoter that is linked to a polynucleotide sequence encoding a protein in such a way that the promoter controls expression of the nucleotide sequence encoding the protein.

The expression cassette of the invention may further comprise other regulatory sequences required for suitable expression of the recombinant protein of interest in the host cell, for example, 5′ untranslated region, signal peptide, polyadenylation region and/or other 3′ untranslated regions.

### 4.1 Iron-Regulated Promoters and Fur Regulated Promoters

In one specific embodiment, said iron-regulated promoter that can be used in the expression cassette of the invention described herein in paragraph 4 can be any bacterial promoter that is partially or fully transcriptionally repressed by a protein that is selected among the ferric upstream repressor (Fur) or homologs of Fur repressor proteins that function in response to the availability of iron in the culture medium. It further includes any promoter that contains a Fur repressor binding site that can be operatively linked to a coding sequence so that it controls expression of such coding sequence in Fur-dependent manner and in response to the availability of iron in the culture medium. Examples of bacterial Fur repressor proteins are known in the art and are described for example in Carpenter et al. (2009).

As used herein a promoter is repressed in response to an external stimuli or a cis-element or a repressor if the promoter activity under repressed conditions (i.e. in the presence of repressor or repressor stimuli and/or repressor binding site) is at least 5 fold lower than the promoter activity under derepressed conditions (i.e. in the absence of repressor or repressor stimuli and/or repressor binding site), as measured with a reporter gene assay such as lacZ reporter gene assay.

Fur-repressor binding sites are known in the art and have been found in many bacterial species such as E. coli, Pseudomonas aeruginosa, Salmonella typhimurium and Bacillus subtilis (Carpenter et al. (2002). Other Fur-repressor binding sites may be searched by homology to the Fur repressor binding site consensus sequence of SEQ ID NO:64. In preferred embodiments, a Fur-repressor binding site is selected among the group consisting of any one of SEQ ID NOs:64-68.

Fur-regulated promoters are known in the art and have been identified in many bacterial species such as E. coli, Pseudomonas aeruginosa, Vibrio cholera, Salmonella typhimurium, Bacillus subtilis, Helicobacter pylorii, Mycobacterium tuberculosis, Bradyrhizobium japonicum, Listeria monocytogenes, Campylobacter jejuni, Streptomyces coelicolor, Yersinia pestis and Staphylococcus aureus (Carpenter et al. (2002)). Examples of Fur-regulated promoters includes without limitations any one of SEQ ID NOs:69-71.

In preferred embodiments, a Fur-regulated promoter is a polynucleotide sequence selected among the group consisting of:

a) SEQ ID NO:69

b) a fragment of SEQ ID NO:69 retaining substantially the same promoter activity as SEQ ID NO:69,

c) a variant promoter of SEQ ID NO:69 with at least 50%, 60%, 70%, 80%, 90% or 95% identity to SEQ ID NO:69.

In one embodiment, a fragment of SEQ ID NO:69 is a fragment that contains at least one Fur-repressor binding site of SEQ ID NO:65 or SEQ ID NO:66 and any 3′ downstream sequences of SEQ ID NO:69.

In some embodiment, said variant promoter may be a nucleic acid containing Fur-repressor binding sites identical to SEQ ID NO:65 or SEQ ID NO:66 or with no more than 1, 2, 3, 4 or 5 nucleotide changes in any one of the Fur-repressor binding sites of SEQ ID NO:65 and SEQ ID NO:66.

In another embodiment, said variant promoter of SEQ ID NO:69 is a functional variant that retains substantially the same activity as SEQ ID NO:69. In a specific embodiment, said variant promoter is a functional variant that retains substantially the same activity as SEQ ID NO:69 and is at least 50% identical to SEQ ID NO:69 but comprises two repressor binding sites identical to SEQ ID NO:65 and SEQ ID NO:66 respectively, or with no more than 1, 2, 3, 4 or 5 nucleotide changes when aligned with SEQ ID NO:65 and SEQ ID NO:66 respectively.

To determine promoter activity of a promoter and compare with the promoter activity of SEQ ID NO:69, it is possible to use any suitable reporter gene assay, such as lacZ reporter gene assay, and measure the reporter gene expression directly, for example, by measuring mRNA levels, or indirectly by measuring a reporter enzyme activity (such as beta-galactosidase activity) under repressed and derepressed conditions. If such activities under repressed and derepressed conditions do not differ significantly between the tested promoter and the promoter of SEQ ID NO:69, then said test promoter is said to retain substantially the same promoter activity as SEQ ID NO:69.

### 4.2 Expression Vectors and Recombinant Host Cells Comprising the Expression Cassette with Iron-Regulated Promoters

The expression cassette may be inserted into any suitable expression vectors. In the context of the synthesis of recombinant protein of interest using the Fur regulated promoters, an expression vector means a vehicle by which a nucleic acid can be introduced into a host cell, resulting in heterologous expression of the gene encoding the recombinant protein of interest.

It can be derived, e.g., from a plasmid, bacteriophage or cosmid or other artificial chromosomes, or other vectors commonly used for recombinant protein production in a host cell. Such expression vector further comprise in addition to the expression cassette, means for entering into the host cells, and/or replicating in said host cells and/or means for secreting the polypeptide at the surface of the cells or outside of the cells. Expression vectors may also include means for being replicated or propagated in more than one cell type, for example, in at least two cell types, one prokaryotic cell type and one eukaryotic cell type.

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), pGEM1 (Promega Biotec, Madison, Wis., USA) pQE70, pQE60, pQE-9 (Qiagen), pD10, phiX174, pBluescript™ II KS, pNH8A, pNH16a, pNH18A, pNH46A (Stratagene), ptrc99a, pKK223-3, pKK233-3, pDR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and stable in the host cell.

The expression vector may be introduced into the host cells using any of a variety of techniques, including electroporation transformation, transfection, transduction, viral infection, gene guns, or Ti-mediated gene transfer. Where appropriate, the engineered host cells can be cultured in conventional nutrient media modified as appropriate for activating promoters, selecting transformants or amplifying the genes encoding the recombinant protein of interest.

In a further aspect, the recombinant host cell of the present invention is capable of expressing or expresses the recombinant protein of interest. An overview of examples of different expression systems to be used for generating the host cell of the present invention, for example the above-described particular one, is for instance contained in Glorioso et al. (1999), Expression of Recombinant Genes in Eukaryotic Systems, Academic Press Inc., Burlington, USA, Paulina Balbas and Argelia Lorence (2004), Recombinant Gene Expression: Reviews and Protocols, Second Edition: Reviews and Protocols (Methods in Molecular Biology), Humana Press, USA.

The transformation or genetically engineering of the host cell with a nucleotide sequence or the expression vector according to the invention can be carried out by standard methods, as for instance described in Sambrook and Russell (2001), Molecular Cloning: A Laboratory Manual, CSH Press, Cold Spring Harbor, N.Y., USA. Moreover, the recombinant host cell of the present invention is cultured in nutrient media meeting the requirements of the particular host cell used, in particular in respect of the pH value, temperature, salt concentration, aeration, antibiotics, vitamins, trace elements etc.

Generally, the recombinant host cell of the present invention may be a prokaryotic or eukaryotic cell comprising the expression cassette and/or the expression vector of the invention or a cell derived from such a cell and containing the expression cassette of the invention and/or the expression vector of the invention.

The invention therefore relates to a recombinant host cell comprising, either integrated in its genome or as an autonomous replicon, an expression cassette or an expression vector of the invention as described above, for heterologous gene expression, or for the synthesis of a recombinant protein of interest under appropriate growth culture conditions.

The “recombinant host cell” can be any suitable cell for the heterologous expression of the recombinant protein of interest under appropriate growth culture conditions. Preferably such recombinant host cell is a bacterial cell.

In a preferred embodiment, the recombinant host cell is a bacterial host cell which has been transformed or transfected with an expression vector comprising the open reading frame encoding the mature recombinant protein of interest operatively linked to an iron-regulated promoter as described in the above paragraph. In a more specific embodiment, the recombinant host cell is selected among Pseudomonas species, for example Pseudomona putida, most preferably, Pseudomonas putida KT2440, comprising an expression vector of the invention, wherein said iron-regulated promoter is selected among the group consisting of any one of SEQ ID NO:69-71, or any functional variant promoter thereof.

The invention further relates to use of the expression cassette, the expression vectors and/or the recombinant host cells as described above for heterologous gene expression, for example in the synthesis of a recombinant protein of interest.

### 4.3 Methods for Heterologous Gene Expression

A recombinant host cell of the invention containing an iron-regulated promoter can be advantageously used for heterologous gene expression, for example for the synthesis of a recombinant protein of interest. Following transformation of a suitable host cell and growth of the host cell to an appropriate cell density, the Fur regulated promoter may be derepressed by appropriate means (e.g., Fe chelating agent, starvation of Fe) and the cells may be cultured for an additional period to allow them to produce the protein of interest.

Thus, in one embodiment, the invention provides a method for heterologous gene expression, or for the synthesis of a recombinant protein of interest in a host cell, preferably in a bacterial host cell, and more preferably in Pseudomonas species, comprising a) culturing said host cell comprising an expression cassette comprising an iron-regulated promoter, under repressed conditions,

b) changing the growth conditions for derepressing the iron-regulated promoter at an appropriate production stage,

c) growing the cells under derepressed conditions for allowing heterologous gene expression and/or synthesis of the recombinant protein of interest.

In one specific embodiment, repressed conditions are obtained by providing iron at sufficient concentration in the growth medium and derepressed conditions are obtained by creating conditions of iron insufficiency. Such conditions can be reached by natural use and starvation of the iron during growth phase. Alternatively, such conditions can be obtained by adding in the medium an iron chelating agent.

Any suitable iron chelating agent can be used for allowing derepression of iron regulated promoter. Examples of such iron chelating agent includes without limitation ethylenediaminetetraacetic acid (EDTA), citrate or compounds known to act as iron uptake siderophores (such as desferrioxamine, enterobactin or bacillibactin). In one preferred embodiment, such iron chelating agent is 2′2′ dipyridyl. The chelating agent can be added in the medium, for example, at a concentration at least equal to, or preferably at least 3 times higher than the iron concentration in the growth medium.

### 4.4 Specific Embodiments of the Invention Related to the Use of Iron-Regulated Promoters for Heterologous Gene Expression

### Embodiment 1

An expression cassette suitable for heterologous gene expression in a host cell, preferably a bacterial host cell, more preferably Pseudomonas host cell, comprising an iron-regulated promoter operatively linked to gene that is not naturally regulated by said iron-regulated promoter.

### Embodiment 2

The expression cassette according to Embodiment 1, wherein said iron-regulated promoter is a bacterial promoter repressed by a protein selected among the group consisting of ferric uptake regulator repressor proteins (Fur), or any homologous promoter sequence that is transcriptionally repressed by a Fur repressor protein.

### Embodiment 3

The expression cassette according to Embodiment 2, wherein said promoter repressed by a Fur repressor protein is a polynucleotide sequence selected among the group consisting of:

(a) SEQ ID NO:69

(b) a fragment of SEQ ID NO:69 retaining substantially the same promoter activity as SEQ ID NO:69,

(c) a polynucleotide sequence with at least 50% identity to SEQ ID NO:69, retaining substantially the same promoter activity as SEQ ID NO:69.

### Embodiment 4

A recombinant host cell, comprising the expression cassette of any of embodiments 1-3,

### Embodiment 5

The recombinant host cell of Embodiment 4, which is selected among bacterial species.

### Embodiment 6

The recombinant host cell of Embodiment 5, which is selected among Pseudomonas species, for example, Pseudomonas putida.

### Embodiment 7

The use of an iron-regulated promoter for the synthesis of a recombinant protein of interest in a host cell.

### Embodiment 8

The use according to Embodiment 7, wherein said iron-regulated promoter is a bacterial promoter repressed by a protein selected among the group consisting of ferric uptake regulator repressor proteins (Fur) or any homologous promoter sequence that is transcriptionally repressed by a Fur repressor protein.

### Embodiment 9

The use according to Embodiment 7, wherein said promoter repressed by a Fur repressor protein is a polynucleotide sequence selected among the group consisting of:

(a) SEQ ID NO:69

(b) a fragment of SEQ ID NO:69 retaining substantially the same promoter activity as SEQ ID NO:69,

(c) a polynucleotide sequence with at least 50% identity to SEQ ID NO:69, retaining substantially the same promoter activity as SEQ ID NO:69.

### Embodiment 10

The use according to any one of Embodiments 7-9, wherein said synthesis of a recombinant protein of interest is controlled by modulating iron concentration in the growth culture.

### Embodiment 11

The use according to any one of Embodiments 7-10, wherein said synthesis of a recombinant protein of interest is carried out in a bacterial host cell, preferably Pseudomonas species, for example Pseudomonas putida.

### Embodiment 12

The use according to any one of Embodiments 7-11, wherein said synthesis of a recombinant protein of interest is induced by the addition of an iron chelator in the medium at a concentration sufficient to chelate the iron and derepress said iron-regulated promoter.

### Embodiment 13

The use according to Embodiment 12, wherein said iron chelator is 2′2′ dipyridyl.

### 5. Depsipeptides Obtained by Heterologous Expression and their Use

The invention further relates to the compounds of formula (I) or (I′), for example, of formula (II) to (VII), (XI) to (XIV) and (XVII) and (XVIII), obtainable or obtained by the method described above.

In a further aspect, the invention relates to the pharmaceutical composition comprising the compounds of formula (I) or (I′), for example, of formula (II) to (VII), (XI) to (XIV) and (XVII) and (XVIII), obtainable or obtained by the method described above.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The skilled person knows that the effective amount of pharmaceutical composition administered to an individual will, inter alia, depend on the nature of the compound. For example, if said compound is a (poly)peptide or protein the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and for example, for humans between about 0.01 and 1 mg protein/kg/day. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect. The particular amounts may be determined by conventional tests which are well known to the person skilled in the art.

Pharmaceutical compositions of the invention may be administered orally, parenterally, intracisternally, intraperitoneally, topically (as by powders, ointments, drops or transdermal patch), bucally, or as an oral or nasal spray.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained release pharmaceutical composition also include liposomally entrapped compound. Liposomes containing the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

The present invention also relates to the use of the above-described depsipeptides, and derivatives thereof, as a medicament. For instance for the treatment of cancer, in particular ovarian cancer, or for the treatment of inflammatory and/or hyperpoliferative and pruritic skin diseases such as keloids, hypertrophic scars, acne, atopic dermatitis, psoriasis, pustular psoriasis, rosacea, Netherton's syndrome or other pruritic dermatoses such as prurigo nodularis, unspecified itch of the elderly as well as other diseases with epithelial barrier dysfunction such as aged skin, inflammatory bowel disease and Crohn's disease, as well as pancreatitis, or of cancer, in particular ovarian cancer, cystic fibrosis (CF), chronic obstructive pulmonary disease (COPD), pulmonary fibrosis, adult respiratory distress syndrome, chronic bronchitis, hereditary emphysema, rheumatoid arthritis, IBD, psoriasis, asthma.

In one embodiment the present invention relates to the use of the above-described depsipeptides, and derivatives thereof, as a medicament for the treatment of inflammatory and/or hyperpoliferative and pruritic skin diseases such as keloids, hypertrophic scars, acne, atopic dermatitis, psoriasis, pustular psoriasis, rosacea, Netherton's syndrome or other pruritic dermatoses such as prurigo nodularis, unspecified itch of the elderly as well as other diseases with epithelial barrier dysfunction such as aged skin, inflammatory bowel disease and Crohn's disease, as well as pancreatitis, or of cancer, in particular ovarian cancer.

In another embodiment the present invention relates to the use of the above-described depsipeptides, and derivatives thereof, as a medicament for the treatment of cystic fibrosis (CF), chronic obstructive pulmonary disease (COPD), pulmonary fibrosis, adult respiratory distress syndrome, chronic bronchitis, hereditary emphysema, rheumatoid arthritis, IBD, psoriasis, asthma.

In yet another embodiment the present invention relates to the use of the above-described depsipeptides, and derivatives thereof, as a medicament for the treatment of inflammatory and/or hyperpoliferative and pruritic skin diseases such as keloids, hypertrophic scars, acne, atopic dermatitis, psoriasis, pustular psoriasis, rosacea, Netherton's syndrome or other pruritic dermatoses such as prurigo nodularis, unspecified itch of the elderly.

### 6. Antibody Against the Polypeptides of the Invention

In a particular embodiment, the present invention relates to an antibody and the use thereof that specifically binds to the polypeptide of the invention or fragments thereof as described and defined herein. Moreover, said antibody can be used for the purification of said polypeptide, in particular non ribosomal peptide and/or non ribosomal peptide synthases (NRPS). The term “antibody” is well known in the art.

In context of the present invention, the term “antibody” as used herein relates in particular to full immunoglobulin molecules as well as to parts of such immunoglobulin molecules substantially retaining binding specificity. Furthermore, the term relates to modified and/or altered antibody molecules, like chimeric and humanized antibodies, recombinantly or synthetically generated/synthesized antibodies and to intact antibodies as well as to antibody fragments thereof, like, separated light and heavy chains, Fab, Fab/c, Fv, Fab′, F(ab′)₂. The term “antibody” also comprises bifunctional antibodies, trifunctional antibodies and antibody constructs, like single chain Fvs (scFv) or antibody-fusion proteins.

Techniques for the production of antibodies are well known in the art and described, e.g. in Howard and Bethell (2000) Basic Methods in Antibody Production and Characterization, Crc. Pr. Inc. Antibodies directed against a polypeptide according to the present invention can be obtained, e.g., by direct injection of the polypeptide (or a fragment thereof) into an animal or by administering the polypeptide (or a fragment thereof) to an animal. The antibody so obtained will then bind polypeptide (or a fragment thereof) itself. In this manner, even a fragment of the polypeptide can be used to generate antibodies binding the whole polypeptide, as long as said binding is “specific” as defined above.

Particularly preferred in the context of the present invention are monoclonal antibodies. For the preparation of monoclonal antibodies, any technique which provides antibodies produced by continuous cell line cultures can be used. Examples for such techniques include the hybridoma technique, the trioma technique, the human B-cell hybridoma technique and the EBV-hybridoma technique to produce human monoclonal antibodies (Shepherd and Dean (2000), Monoclonal Antibodies: A Practical Approach, Oxford University Press, Goding and Goding (1996), Monoclonal Antibodies: Principles and Practice—Production and Application of Monoclonal Antibodies in Cell Biology, Biochemistry and Immunology, Academic Pr Inc, USA).

The antibody derivatives can also be produced by peptidomimetics. Further, techniques described for the production of single chain antibodies (see, inter alia, U.S. Pat. No. 4,946,778) can be adapted to produce single chain antibodies specifically recognizing the polypeptide of the invention. Also, transgenic animals may be used to express humanized antibodies to the polypeptide of the invention.

The term “specifically binds”, as used herein, refers to a binding reaction that is determinative of the presence of the non ribosomal peptide and/or non ribosomal peptide synthases (NRPS) and antibody in the presence of a heterogeneous population of proteins and other biologics. Thus, under designated assay conditions, the specified antibodies and polypeptides bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background.

The term “purification”, as used herein, refers to a series of processes intended to isolate a single type of protein from a complex mixture. Protein purification is vital for the characterisation of the function, structure and interactions of the protein of interest. The starting material, as a non-limiting example, can be a biological tissue or a microbial culture. The various steps in the purification process may free the protein from a matrix that confines it, separate the protein and non-protein parts of the mixture, and finally separate the desired protein from all other proteins. Separation steps exploit differences in protein size, physico-chemical properties and binding affinity.

The present invention is further described by reference to the following non-limiting figures, sequences and examples.

The figures show:

FIG. 1 shows a list of confirmed structures produced by Chondromyces NPH-MB180 that are biosynthesized from the NRPS cluster according to the present invention.

FIG. 2 shows the domain architecture of the NRPS biosynthetic gene cluster encoding for a compound of formula (I) or (I′), exemplified by proposed biosynthesis route for compounds of formula (II), (III), (VI), and (VII)-(XVII). L, loading domain; AQ adenylation domain (Gln); T thiolation domain; C, condensation domain; NM, N-methylation domain; TE, thioesterase domain, AP adenylation domain (Pro); AT, adenylation domain (Thr); AL, adenylation domain (Leu); AE, adenylation domain (Glu); AI, adenylation domain (Ile); AY, adenylation domain (Tyr).

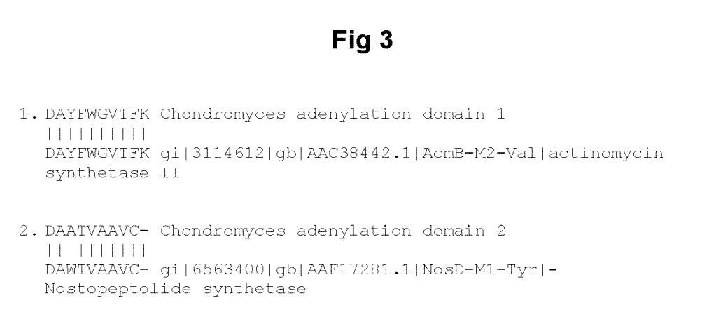

FIG. 3 shows an alignment of the ten amino acid residues that line the binding pocket of the two adenylation domains in the NRPS segment F 10517242 with their closest match to defined adenylation domains.

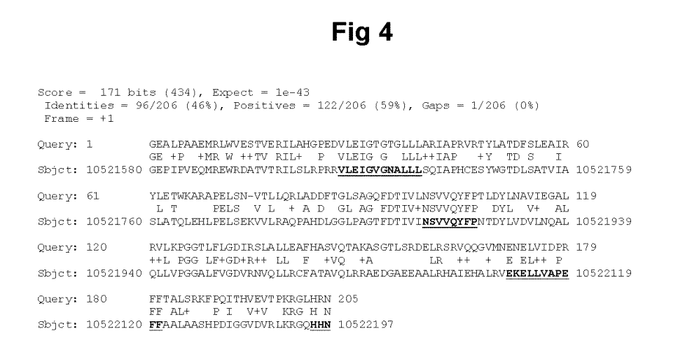

FIG. 4 shows the results from BLASTp alignment of the Chondromide N-methylation domain against the Chondromyces NPH-MB180 which reveal the N-methylation domain located in the NRPS segment F 10517242. N-methylation domain motifs are colored in bold.

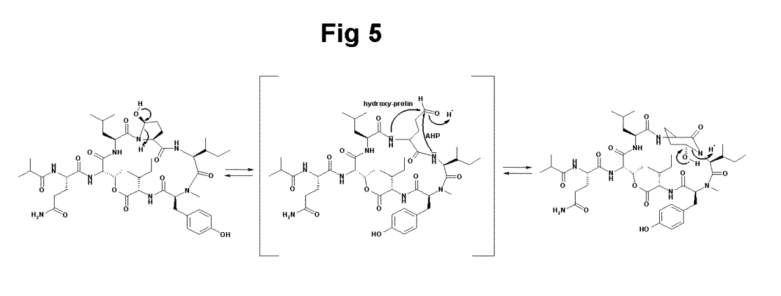

FIG. 5 shows the presumed interconversion of a compound containing hydroxy-proline to form the ahp residue. Under aqueous conditions there is equilibrium between the hydroxyproline exemplified by formula (XVIII) and the ahp containing compound exemplified by formula (II).

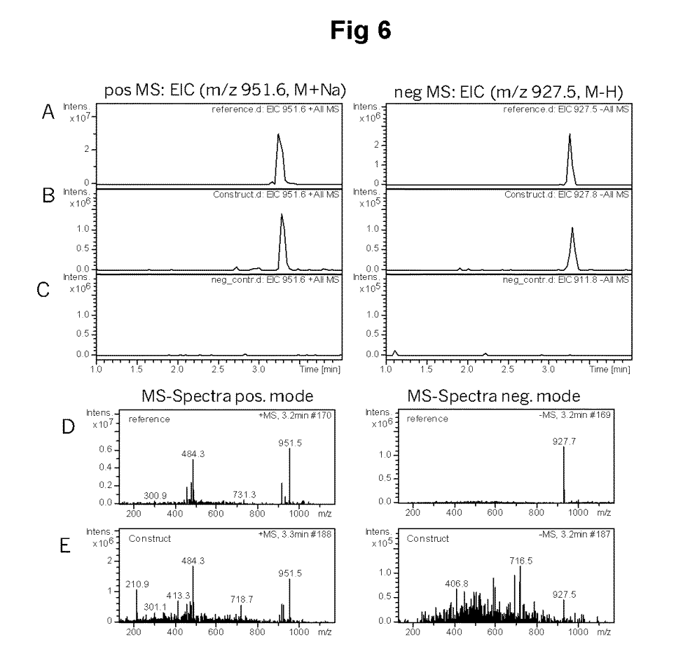

FIG. 6 Detection of compound of formula II by LC-MS analysis of extracts from a heterologous expression culture of P. putida KT2440. HPLC chromatograms showing positive (left panels) and negative (right panels) ion detection by MS: A) formula II reference compound; B) day 6 LB_D medium; C) day 6 P. putida negative control. MS-Spectra: D) formula II reference compound from HPLC run shown in A; E) day 6 LB_D medium peak at 3.2 min from HPLC run shown in B.

The present invention refers to the following nucleotide and amino acid sequences:

SEQ ID NO: 1 depicts the nucleotide sequence encoding the amino acid sequence of Domain 1, representing the Val/Ile condensation domain.

SEQ ID NO: 2 depicts the amino acid sequence of Domain 1, representing the Val/Ile condensation domain.

SEQ ID NO: 3 depicts the nucleotide sequence encoding the amino acid sequence of Domain 2, representing the Val/Ile adenylation domain.

SEQ ID NO: 4 depicts the amino acid sequence of Domain 2, representing the Val/Ile adenylation domain.

SEQ ID NO: 5 depicts the nucleotide sequence encoding the amino acid sequence of Domain 3, representing the Val/Ile thiolation domain.

SEQ ID NO: 6 depicts the amino acid sequence of Domain 3, representing the Val/Ile thiolation domain.

SEQ ID NO: 7 depicts the nucleotide sequence encoding the amino acid sequence of Domain 4, representing the Tyr condensation domain.

SEQ ID NO: 8 depicts the amino acid sequence of Domain 4, representing the Tyr condensation domain.

SEQ ID NO: 9 depicts the nucleotide sequence encoding the amino acid sequence of Domain 5, representing the Tyr adenylation domain.

SEQ ID NO: 10 depicts the amino acid sequence of Domain 5, representing the Tyr adenylation domain.

SEQ ID NO: 11 depicts the nucleotide sequence encoding the amino acid sequence of Domain 6, representing the Tyr 6-N-methylation domain.

SEQ ID NO: 12 depicts the amino acid sequence of Domain 6, representing the Tyr 6-N-methylation domain.

SEQ ID NO: 13 depicts the nucleotide sequence encoding the amino acid sequence of Domain 7, representing the Tyr thiolation domain.

SEQ ID NO: 14 depicts the amino acid sequence of Domain 7, representing the Tyr thiolation domain.

SEQ ID NO: 15 depicts the nucleotide sequence encoding a NRPS fragment comprising the adenylation domain, the condensation domain and the thiolation domain for Val/Ile and Tyr, respectively and the Tyr 6-N-methylation domain.

The function and putative role of nucleotide and amino acid sequences described in the present application are further described in Table 1 above and the examples below.

### EXAMPLES

The following Examples illustrate the invention:

### Example 1

### Genome Sequence of NPH-MB180; Assembly and Analysis

The complete genome of NPH-MB180 was sequenced using the 454 sequencing method (a pyrophosphate based sequencing platform) to produce a “draft” sequence. One shotgun sequencing run was performed followed by two paired-end sequencing runs. Paired end runs are used as a complementary technique to the more traditional shotgun method. In brief, they are sequencing runs of physically shredded and circularized chromosomal DNA fragments ligated onto a short DNA adapter section. This permits divergent sequencing out from the adapter giving two short reads (˜150-200 bp) that are located approximately 3 kb apart from each other (average size of shredded circularized DNA). Overlap (homology) of the two short reads on two separate contigs allows for non-overlapping contigs to be linked together and joined by stretches of undefined nucleotides (N) with an approximate length estimated based on the 3 kb approximation. Contigs linked in this manner are termed scaffolds. Overall, 1,295,834 individual reads were performed resulting in 310,674,400 bases sequenced. The average read length was 239 bases; typical for this type of sequencing method. These reads were assembled to form contigs based on sequence overlap between reads. This effort resulted in 4,038 contigs accounting for 15,449,316 bases with an average contig length of 8,931 bp. The use of paired end run overlap to produce scaffolds resulted in the assembly of 96 scaffolds comprising 15,029,556 bases. The average scaffold size was 1,227,671 bases and the average scaffold size was 156,557 bases.

The genome data was analyzed for the purpose of identifying the NRPS gene cluster responsible for the biosynthesis of the depsipeptides of formula (I) or (I′). The overall approach was to use BLAST searches (Altschul et. al. 1990; Gish, W. & States, D. J. 1993) against the 96 scaffolds using NRPS domains as search queries. The NRPS domains relied upon were the adenylation domains, as these domains specify which amino acid is incorporated into the non-ribosomal peptide and therefore are good markers to identify a specific NRPS cluster (Marahiel, M. A. et. al. 1997). It was generally expected to find an NRPS cluster that contained within its architecture adenylation domains with the following specificity and relative order: Gln-Thr-Val-Glu-Ile-Tyr+(N-meth.)-Ile. Furthermore, the gene cluster was expected to start with a loading domain capable of initiating the biosynthesis with a carboxylic acid such as isobutyric acid and further anticipated that the cluster would end with a thioesterase domain. There was also a possibility that other biosynthetic units could be present that facilitate the oxidation of the glutamate residue to form the 3-amino-6-hydroxy-piperidone residue (Ahp). The relative location of these accessory genes, if present in this cluster, was unpredictable. In addition, the location of transcriptional starts and stops to define the one or more transcripts present in the region were unpredictable at this stage.

### Example 2

### Identification of all NRPS Adenylation Domains in NPH-MB180 Genome Sequence by BLAST Analysis

The approach relied on to identify the NRPS cluster was to first identify all NRPS adenylation domains in the NPH-MB180 genome. NRPS adenylation domains are specific for the amino acids that they utilize and therefore these domains were analyzed to identify the correct NRPS cluster based on the content and relative of order of the amino acids that constitute the depsipetides of formula (I) or (I′). Towards this end, the cyclosporine valine adenylation domain was the domain we utilized as an example of a general adenylation domain to identify all possible NRPS clusters in the genome sequence data. This was accomplished by performing a tBLASTn (Altschul et. al. 1990; Gish, W. & States, D. J. 1993) analysis of the genome to identify all NRPS adenylation domains by amino acid sequence homology. This approach identified 14 possible NRPS clusters (Table 2) and the scaffolds containing these clusters were labeled A-N together with the nucleotide number of the start of the original BLAST hit (e.g. A 12171827). From this list each adenylation domain was identified and each domain's specificity was determined by analysis of the conserved amino acid residues that define the domain specificity (see Example 3 for details).

TABLE 2
NRPS containing scaffolds and description
of adenylation domain predictions.

| # | Scaffold Code | Comments/Conclusions | Aden. Dom. Spec. |
|---|---|---|---|
| 1 | A 13171827 | Probable PKS/NRPS | Tip-Ile |
| 2 | B 13514151 | Small NRPS | ? |
| 3 | C 3116250 | Small PKS/NRPS | ?-Leu |
| 4 | D 942267 | NRPS | ?-Thr-Leu; Pro-Val |
| 5 | E 7662639 | NRPS | Tyr; Val-Leu-Ile |
| 6 | F 10517242 | Partial depsipeptide NRPS | Val-Tyr(N-meth) |
| 7 | G 8545357 | NRPS | Cys-?-Ser; Cys-Ser- |
| | | | Asn |
| 8 | H 2301347 | Probable Chondromide | Phe/Trp(N-meth) |
| | | NRPS/PKS | |
| 9 | I 9968425 | Hybrid NRPS/PKS | Thr (pK530) |
| 10 | J 5758635 | Hybrid NRPS/PKS | Gly/Lys |
| 11 | K 10007171 | Hybrid NRPS/PKS | Tyr |
| 12 | L 9213891 | Hybrid NRPS/PKS | Gly |
| 13 | M 13479002 | Probable Hybrid NRPS/PKS | ?-Phe/Trp |
| 14 | N 2469863 | Very small Cys aden. domain. | |

This first analysis failed to identify any NRPS clusters with the correct adenylation domain composition and overall size of the expected cluster (˜30 kb) to code for the biosynthetic pathway. In fact, no NRPS pathway was identified that contained seven adenylation domains as we would expect to find in our pathway of interest. It was, however, noted that F 10517242 contained isoleucine and tyrosine adenylation domains (Table 2). Incidentally, this scaffold (scaffold #72) is quite short (˜7.4 kb) but it was hypothesized that this is a portion of the NRPS cluster of interest and that the remainder of the cluster remains unsequenced (resides in sequencing gap regions). The discovery of an N-methylation domain residing between the tyrosine adenylation domain and the partial T domain provided additional support for this hypothesis (see Example 4 for details).

Based on these data it was concluded that the genome sequence does not contain the biosynthetic gene cluster in its entirety. Indeed it can be predicted that approximately 20 kb in the 5′ direction and 6 kb in the 3′ direction remain unaccounted for.

### Example 3

### Prediction of NRPS Adenylation Domain Specificity

The specificity of the adenylation domains described herein is predicted using the following general protocol. The adenylation domains were identified using a tBLASTn (Altschul et. al. 1990; Gish, W. & States, D. J. 1993) search that aligned the amino acid sequence of the valine adenylation domain of cyclosporin synthase (CssA) against the Chondromyces genomic DNA of interest. Using ClustaIX multiple sequence alignment software (Higgins et. al. 1996) the translated Chondromyces adenylation domain was aligned against GrsA (PheA) (Gramicidin S synthetase) at the amino acid level between the two core motifs (A3 and A6) defined by Marahiel et. al. (1997). The ten amino acids reported by Marahiel et al. that define the binding pocket of the adenylation domain and therefore dictate the amino acid specificity were identified in this alignment. The ten amino acids were then compared with defined adenylation domain amino acid codes using data reported by Rausch et. al. (2005) and Stachelhaus et. al. (1999).

The two adenylation domains identified in the segment of the biosynthetic cluster showed high homology to the ten amino acids that define the binding pockets for isoleucine and tyrosine (FIG. 3). These amino acid specificities are not absolute and amino acids with similar chemical characteristics are often substituted in place of the amino acid that defines the domain. This accounts for the variability in structures that are synthesized off of one NRPS operon. In the present case, it is assumed that the isoleucine adenylation domain can also accept valine into its binding pocket, a characteristic that has been shown for other “isoleucine” adenylation domains Rausch et. al. (2005). Indeed, available NRPS prediction tools (e.g. http://www-ab.informatik.uni-tuebingen.de/software/NRPSpredictor) are generally unable to declare an adenylation domain as isoleucine specific or valine specific.

### Example 4

### Prediction of NRPS N-Methylation Domains

The presence of an N-methylation domain was predicted to be located directly adjacent to the tyrosine adenylation domain in the 3′ direction using the following approach. The amino acid sequence for the N-methylation domain of the Chondromyces crocatus NPH-MB180 Chondromide NRPS cluster was utilized to search the genome for similar domains using tBLASTn (Altschul et. al. 1990; Gish, W. & States, D. J. 1993). Using this approach an N-methylation domain was identified within the NRPS segment that had an Expect value of 5e-43 and 46% amino acid sequence identity (FIG. 4). In addition, it was noted that the N-methylation domain from this the NRPS segment possessed expected amino acid motifs that are commonly found in functional N-methylation domains (von Dohren, H. et. al. 1997; Marahiel, M. A. et. al. 1997). To confirm this data, the N-methyltransferase Apsy-6 from the Anabaena strain 90 anabaenopeptilide biosynthetic cluster (Rouhiainen et. al. 2000) was compared to the N-methylatransferase described above. The BLASTp results of this comparison reveal that these domains are highly homologous with an Expect value of 2e-65 thereby confirming the initial identification of this domain. The presence of this domain directly adjacent to the tyrosine adenylation domain is consistent with the expected architecture of the NRPS gene cluster. Furthermore, N-methylation domains are relatively uncommon, and therefore the presence of this domain within the NRPS segment provides strong evidence for this segment belonging to the NRPS clusters.

### Example 5

### Identification of the Entire Biosynthetic NRPS Gene Cluster

The complete nonribosomal peptide biosynthetic genes responsible for production of depsipeptides of formula (I′) was identified and characterized. The biosynthetic genes were assembled onto a scaffold composed of scaffold F 10517242 inserted into scaffold D 942267 (Table 1). The combination of these scaffolds was performed after sequence analysis of the nucleotides directly adjacent to scaffold F 10517242 indicated that this insertional adjustment to the original genome assembly was warranted. This assemblage has been confirmed by PCR with subsequent DNA sequencing through the scaffold joining regions. Within this scaffold are eight contiguous open reading frames that are likely responsible for the biosynthesis, modification and extracellular export of the depsipeptides of formula (I′). In addition, a possible secreted protease is located within these open reading frames that may ultimately be the natural cellular target of the depsipeptides, demonstrated protease inhibitors. The arrangement of these ORFs and corresponding NRPS domains is shown in FIG. 2.

Directly in front of the core nonribosomal peptide open reading frames (ORF6 and ORF7) are five ORFs. ORF1 and ORF2 are each homologous to two different uncharacterized proteins reported from Sorangium cellulosum. These proteins have no hypothetical function, however it is noteworthy that they appear to be found only in the family Polyangiaceae. Furthermore, the Sorangium proteins that are homologous to ORF2 are found at least five times in the S. cellulosum genome. These proteins appear to be co-transcribed with ORF3 based on their near perfect nucleotide sequence contiguity. ORF3 has high sequence homology with serine proteases, in particular those belonging to the subtilisin group. We have determined biochemically that depsipeptides are highly specific serine protease inhibitors and it is therefore plausible that depsipeptides are an inhibitor of the ORF3 serine protease. Conversely, ORF3 may be involved with imparting depsipeptide resistance to the Chondromyces strain. ORF4 and ORF5 are homologous to siderophore permeases and general cyclic peptide permeases, of the ABC transporter type. It is likely that this permease system is involved with the export of depsipeptides across the cytoplasmic membrane. In fact, it is possible that all five of these ORFs are involved with a cytoplasmic membrane translocation process and that the “serine protease-like” ORF3 shares similarity with the serine protease family only because, as with actual proteases, it binds the protease inhibitor.

The core depsipeptide biosynthetic cluster begins with ORF6 and continues through ORF7. These two ORFs combined are over 15 kb in length. As with all NRPS biosynthetic clusters they can be broken down into functional domains that have a general topology consisting of a condensation domain followed by an adenylation domain followed by a thiolation domain (Marahiel et. al. 1997). This three domain module is usually repeated multiple times in an NRPS cluster, once for each amino acid incorporated into the peptide. The depsipeptide biosynthetic cluster follows this pattern with seven such modular repeats to account for the seven amino acids contained in the peptide core. Adenylation domains confer amino acid specificity to the growing peptide and can be analyzed to identify the amino acids that they accept and subsequently incorporate.

The predicted amino acid specificities of the seven adenylation domains present in ORFs 6 and 7 are in general agreement with the final structure of depsipeptides with one exception. The fourth adenylation domain (domain 7.3) is predicted to accept and incorporate proline into the growing peptide at this position while the final peptide contains a non-standard amino acid, 3-amino-6-hydroxy-piperidone (ahp), in this position. Ahp is present in several depsipeptides, including the related anabaenapeptolides produced by Anabaena strain 90 (Rouhiainen et. al. 2000). It has been postulated that ahp formation occurs in anabaenapeptolides after glutamine is incorporated into position four of the chain which then reacts back on the amine of the previous amino acid to form ahp (Rouhiainen et. al. 2000). However ahp specific adenylation domains have also been described in the literature (Rausch et al. 2005). In ahp containing depsipeptides isolated from strain MB180 of formula II-VII, XI to XIII and XVII, we now presume a novel process of ahp formation, in which proline is initially incorporated into the growing peptide in position four and ahp is subsequently formed with the aid of an oxidoreductase. Indeed a cytochrome P450 gene (ORF8) has been surprisingly found in the depsipeptide biosynthetic cluster, it is located immediately after the NRPS biosynthetic cluster and likely catalyzes the conversion by hydroxylating the proline residue.

It is noteworthy that depsipeptides analogs that contain proline at this position have been isolated from strain MB180 (formula XIV). It was also demonstrated that analogs with a 5-hydroxyproline (formula XVIII) form spontaneously from ahp containing depsipeptides (for example formula II) upon incubation in aqueous environment for several days (FIG. 5). This interconversion between the 5-hydroxyproline form and the ahp form has also been shown by us to be reversible. While it is unclear whether other depsipeptides also follow this strategy it is likely that this is the ahp formation strategy employed by our strain MB180.

The depsipeptide biosynthetic cluster begins in ORF6 with a loading domain that initiates the biosynthesis with a starter unit. As starter unit carboxylic acids such as CH₃CH₂CH(CH₃)COOH, (CH₃)₂CHCOOH, C₆H₅COOH, CH₃S(O)CH₂COOH or CH₃COOH can be postulated based on the structural variation of the X residues in depsipeptides of formula (I′).

While it is common for nonribosomal peptides to initiate with a small acid residue the choice of residue differs considerably from peptide to peptide. However, complex carboxylic acid starter units are relatively uncommon among nonribosomal peptides. The loading domain utilized to initiate depsipeptide biosynthesis is different from the anabaenapeptolide loading domain both structurally and in the starter unit employed. In fact the depsipeptide loading is very closely related to a standard condensation domain while the formyl group loading domain of anabaenapeptolide closely resembles previously described formyl transferases (Rouhiainen et. al. 2000). After the carboxylic acid starter unit is condensed onto the alpha amino group of the glutamine amino acid specified by domain 6.2, the chain continues to grow one amino acid at a time as it proceeds sequentially through the NRPS biosynthetic apparatus (FIG. 2).

The depsipeptide biosynthetic apparatus synthesizes the peptide one amino acid at a time without deviation from a simple NRPS peptide until it encounters a relatively rare methyl transferase domain (domain 7.10) which methylates the secondary amine of a peptide bond. In this case this results in a tertiary amine on the tyrosine derived amino group. Presumably this methylation occurs after the tyrosine is added to the growing peptide but before the next and final amino acid is added. This is strongly suggested by the location of the N-methylase domain immediately following the tyrosine specific adenylation domain.

Finally, the peptide is removed from the final thiolation domain and cyclized forming an ester bond between the threonine alcohol and the alpha keto group of the terminal isoleucine. This is performed by a standard thioesterase domain (domain 7.15) that is the final domain located in ORF7. It is unclear if ahp formation occurs before or after this thioesterase step. Regardless, the genes contained within this biosynthetic cluster are sufficient to account for the entire structure of the depsipeptides of formula (I′).

### Example 6

### Heterologous Expression of Depsipeptide in Pseudomonas putida KT2440

Here we describe one example of an approach to achieve heterologous expression of the depsipeptide of formula (I) or (I′), in Pseudomonas putida KT2440. This host has several advantages over the native producer strain C. crocatus including rapid and predictable growth, the availability of genetic tools and validated use in large scale fermentation. In addition, this host has a genomic GC % similar to C. crocatus and possesses native NRPS systems; two traits which are important considerations when designing heterologous expression strategies.

The biosynthetic gene cluster was cloned into the cosmid pWEB-TNC (Epicenter Biotechnologies, Madison Wis., USA) which is able to accept large inserts; an essential quality given that the biosynthetic gene cluster exceeds 30 kb in length. Cloning of the biosynthetic gene cluster was performed by first identifying an appropriate restriction enzyme that would cut outside the boundaries of the biosynthetic cluster to generate a linear DNA fragment of approximately 30-40 kb. Analysis of the genome sequence data revealed that the enzyme XmnI was appropriate for this task and would generate 15 different DNA fragments in this size range when a complete genomic DNA digestion was performed. Of these 15 DNA fragments, one 39 kb fragment was predicted to contain the biosynthetic cluster. These 15 DNA fragments were separated from the other chromosomal digest fragments by agarose gel electrophoresis. The 15 DNA fragments in the desired size range were gel excised using appropriately sized DNA standards as a guide and cloned into the cosmid pWEB-TNC according to the manufacturer's instructions. A cosmid clone containing the complete biosynthetic cluster was identified by colony PCR and confirmed by DNA sequencing. An alternative approach could have been to generate a random shotgun library of the complete genome using a cosmid or BAC vector with subsequent colony hybridization to the clone library using a radiolabeled probe to identify the clone library member that contained the biosynthetic cluster of interest.

After obtaining the cloned biosynthetic pathway several genetic components were required to be inserted into the cosmid clone to permit successful heterologous expression. These components included i) a selectable marker to permit identification of successful transfers into the heterologous host, ii) a promoter that functions in the heterologous host, iii) a site for chromosomal integration into the heterologous host and iv) plasmid conjugal transferability functions conferred by the pRK2013 oriT sequence (for use with RK2 transfer functions). The selectable marker we chose for use in Pseudomonas putida KT2440 for this example was the gentamicin resistance cassette aacCl (Blondelet-Rouault et al. 1997). Other selectable markers could have included nucleotide cassettes that confer resistance to ampicillin (such as bla), chloramphenicol (such as cat), kanamycin (such as aacC2, aadB or other aminoglycoside modifying enzymes) or tetracycline (such as tetA and tetB). As a promoter to drive heterologous expression in Pseudomonas putida KT2440, we describe here the use of the fumarase C-1 (PP 0944) gene promoter (see also Example 8). The choice of transcriptional promoters could include the transcriptional promoters of any of the above listed antibiotic resistance determinants or any transcriptional promoter that is functional in Pseudomonas putida KT2440 including, but not limited to, the transcriptional promoters of the seven 16S rRNA genes present in the Pseudomonas putida KT2440 genome (PP 16SA, PP 16SB, PP 16SC, PP 16SD, PP 16SE, PP 16SF, PP 16SG), the transcriptional promoters of any Pseudomonas putida KT2440 ferric uptake repressor (Fur) regulated gene, (including the promoters of fagA (PP 0943) or the other fumC homolog, fumC-2 [PP 1755]) the promoters involved in biosynthesis and transport of siderophore or siderophore-like compounds (including pvdE [PP 4216], fpvA [PP 4217]) or the transcriptional promoters for the genes PP 4243 or PP 0946. Promoters from P. putida, including the use of the fumarase C-1 promoter described here, serve a second purpose in our strategy by providing a site of chromosomal integration into the P. putida host via a RecA mediated chromosomal integration event. To facilitate efficient chromosomal integration 1046 bp of the promoter region were included in the cosmid construct. The promoter element was located at the 3′ end of the intended insert to permit the promotion of transcription into the downstream biosynthetic cluster genes. Plasmid conjugation was facilitated through the incorporation of the oriT nucleotide sequence from pSET152. The oriT sequence is necessary and sufficient to permit successful conjugal transfer of the cosmid when RK2 transfer functions are provided in trans. These three genetic components were cloned sequentially (5′-gentamicin resistance-oriT-fumC1 promoter-3′) using pUC19 as a backbone. This heterologous expression cassette was made using standard molecular biological practices.

Once completed the heterologous expression cassette was transferred from pUC19 into the cosmid clone containing the biosynthetic gene cluster. This insertion was performed such that the 3′ terminus of the insert which contains the promoter element was positioned 20 base pairs away from the translational start codon of the first open reading frame of the biosynthetic gene cluster thereby generating a transcriptional fusion of the promoter element to the biosynthetic gene cluster. The promoter was intended to drive transcription of the gene cluster and rely on the native ribosomal binding sites located within the biosynthetic gene cluster to initiate translation of the biosynthetic proteins. This insertion was performed through the use of homologous recombination mediated by the lambda RED recombinase functions according to Chaveroche et al. 2000. Briefly, PCR products were generated that consisted of the construct described above with 100 nt flanks (designed into the PCR primers) with homology to the intended insertion site in the biosynthetic gene cluster. These 100 nt flanks were further extended by adding PCR generated flanks 600 nt in length to the existent 100 nt flanks by long flanking homology PCR (Moore et al. 2005). The heterologous expression cassette with 600 nt homology flanks was electroporated into E. coli EPI100 electrocompetant cells that had previously expressed the lambda RED proteins from the plasmid pKOBEGhyg (a hygromycin cassette containing construct of the pKOBEG plasmid cloned into the HindIII restriction site). Transconjugates that had successfully integrated into the cosmid were selected on Lauria broth agar supplemented with 15 μg/ml gentamicin. The heterologous expression construct thus generated was confirmed by PCR and DNA sequencing. Although less efficient, the insertion of the heterologous expression cassette into the cosmid clone may alternatively be performed by traditional restriction enzyme based cloning strategies.

The heterologous expression construct was conjugally transferred into Pseudomonas putida KT2440 by tri-parental conjugation using established methods (Stanisich and Holloway, 1969) that rely on the E. coli helper strain HB101 (pRK2013) to provide the RK2 transfer functions. P. putida transconjugates were selected on Lauria Broth agar supplemented with 75 μg/ml gentamicin to select for P. putida transconjugates and 25 μg/ml irgasan to prevent E. coli donor and helper strain growth. Transconjugates that had successfully integrated into the P. putida chromosome at the fumC-1 upstream promoter region were confirmed by PCR, Southern hybridization and DNA sequence analysis.

Production of the compound of formula II was confirmed by growth in Lauria Broth containing 2 g/L isobutyric acid and 100 μM 2,2, dipyridyl (medium pH adjusted to 7.0) grown at 15° C. with constant rotational shaking at 200 rpm. Chemical extraction was conducted at day 6 on 5 mL of crude fermentation broth with 1:1 ethyl acetate, followed by concentration to dryness at 30° C. and subsequent reconstitution in methanol to a 20× final concentration. Analysis was performed by HPLC separation using a C-18 column coupled to online DAD, MS and MS/MS detection. Compound of formula II was unambiguously identified using MS and MS/MS detection (FIG. 6).

### Example 7

### Mechanism of Rearrangement of 5-hydroxyproline into 3-amino-6-hydroxy-2-piperidone (ahp)

The core biosynthetic pathway of depsipeptides of formula (I′) suggests that proline is incorporated into the depsipeptide chain at amino acid position 4. This is in line with compound of formula (XIV), which contains a proline instead of ahp or dehydro-ahp. We have identified a cytochrome P450 enzyme (orf 8) which we hypothesize hydroxylates the proline thereby generating compound with 5-hydroxyproline exemplified by formula (XVIII). Compound of formula (XVIII) forms spontaneously from ahp containing depsipeptides (for example formula II) upon incubation in aqueous environment for several days (FIG. 5). This interconversion between the 5-hydroxyproline form and the ahp form has also been shown by us to be reversible and achieves an approximate 9:1 (ahp:5-hydroxyproline) molar ratio equilibrium after 10 days in water at 50° C.

### Example 8

### Use of the Fur Regulated fumC-1 Promoter from Pseudomonas putida KT2440 for Heterologous Gene Expression of the Gene Cluster for the Biosynthesis of Depsipeptides

To be able to successfully heterologously express the biosynthetic gene cluster for depsipeptides in the host Pseudomonas putida KT2440, it was necessary to find a suitable promoter to place in front of the gene cluster in the heterologous host. A fur-regulated promoter from the heterologous host, Pseudomonas putida KT2440 was selected (SEQ ID NO:69). In many, if not most bacteria the transition stage of growth coincides with the onset of iron limitation in the growth media when standard complex growth medium (such as LB) are used. We believed that it would be advantageous to delay the transcription of the biosynthetic gene cluster for depsipeptides in a heterologous host until the transition stage of growth to enable the host to attain a healthy population density and because it is known that most secondary metabolites, in general, are produced at this stage of growth. Genes that are activated in response to iron limitation are often regulated by the ferric uptake repressor (Fur). This metaloregulator acts as a Fe sensor that represses a set of genes under conditions of Fe sufficiency by directly binding to the promoter regions of the regulated genes, thereby physically preventing RNA polymerase binding (Barton et. al. 1996). Under conditions of iron insufficiency Fur releases from the promoter region thus allowing transcription of the genes to occur. Therefore, the use of a Fur-regulated promoter would allow us to repress the expression of the heterologous genes until the transition stage.

We identified potential Fur regulated genes in Pseudomonas putida KT2440 from the published proteome of genes expressed in response to low iron levels relative to sufficient iron levels (Heim et al. 2003) and searched the promoter regions in front of those genes using the Pseudomonas aeruginosa Fur repressor consensus site “gataatgataatcattatc” (SEQ ID NO:64) Barton et al. 1996). One of the most highly up-regulated gene products in Pseudomonas putida KT2440, as determined by the study of the iron regulated proteome from Barton et al, was the gene product for fumC-1 encoding one of the two P. putida fumarase enzymes. Further investigation revealed that this gene had previously been shown to be Fur regulated (Hassett et. al. 1997). We therefore were hoping that this promoter region was strong based on the published data and would act in an iron dependent manner, turning on when iron levels were low in the cell. These characteristics made the fumC-1 promoter region an ideal candidate to use for the purposes of heterologous gene expression in Pseudomonas putida KT2440. The successful heterologous gene expression of the whole biosynthetic gene cluster as shown in the Example 6 and FIG. 6 above confirmed such assumption.

Conditions of iron insufficiency can be obtained in a fermentation culture by adding the iron chelating agent 2′2′ dipyridyl at molar levels equal to or greater than 3× the iron concentration in the fermentation growth medium. This permits Fur regulated genes to be up-regulated in a controlled manner through the addition of 2′2′ dipyridyl. For example, we have used 300 μM 2′2′ dipyridyl in our heterologous expression fermentation cultures using the growth media LB. Other iron chelating agents such as ethylenediaminetetraacetic acid (EDTA), citrate, or compounds known to act as iron uptake siderophores (such as desferrioxamine, enterobactin or bacillibactin) could also be used in a similar manner to create conditions of iron insufficiency in fermentation medium. Alternatively, iron levels could be carefully controlled through the use of defined fermentation medium.

Other Fur regulated promoters could be used in the same manner as we have described here for the successful use of the fumC-1 promoter. For example, promoters controlling the expression of FpvA and OmpR-1 could be used as likely comprising Fur repressor binding sites. Such promoters are further described in detail in Example 9 below. Other Fur binding sites in front of any genes that are up-regulated under conditions of Fe insufficiency could be identified using the bioinformatic approach described here or by using electrophoretic mobility shift assays of purified Fur protein to the DNA of the promoter regions as has been described by Baichoo et al. (2002). The Fur family is wide-spread in the bacterial domain and promoter regions and their respective Fur binding sites are, in general, genus specific and often species specific. As such, it is anticipated that Pseudomonas putida KT2440 Fur regulated promoter regions will also be functional in other Pseudomanas species.

### Example 9

### Fur Regulated Promoters

Fur regulated promoters from Pseudomonas putida KT2440. Fur repressor binding sites are underlined and were identified by consensus nucleotide similarity search against the Pseudomonas aeruginosa Fur repressor consensus site gataatgataatcattatc (SEQ ID NO:64) (Barton et al. 1996).

fumC-1 Fur regulated promoter region (Fur
repressor sites underlined)
(SEQ ID NO: 69)
atcaggccgcgctgattcgccgtatggggcgcgggctgctggtgaccgaa
ctgatggggcatggcttgaacatggtgacgggggactattcccgtggtgc
ggcggggttctgggtcgagaatggcgagattcagcatgccgtacaggaag
tcaccatcgccggaaacatgaaggacatgttccagcagattgtcgcgatc
ggtagcgatcttgaaacccgtagcaatattcatacgggctcggtgttgat
cgagcggatgaccgttgctggtagctgatctttagcctgcgccggccctt
tcgcgggtaaacccgctcctacacggtggtggacgtacatcggggttgga
cacaggccgttgtaggagcgggttcacccgcgaagaggccggaacagcac
tacacctttccctgcaaatccgaagacccggccctcgcgccgggttttta
tttcatcacctttttcttgaagtgattctatttatcacttaataatgaat
atcattatccagtaacccggcgatgatgttcatgaaatccgtcctccgcg
aactgccctacctggaaaactggcgctggctcagccggcgcattcgctgt
gcgctcgaccccgacgagccgcgcctgatcgagcattacctggccgaagg
ccgctatctggtgtgctgcaccgaaacctcgccatggacggtggcgctga
cagcgtttcgcctgctgctggataccgcctgcgatcgcatgctcccctgg
cattggcgttgtctgtgcctggaccaggcgtggcgccctctgctggacct
gcgcaacctcgaccgccaggaacagaaccaacgctggcaaccctacgcct
tgcagttggccaattgccgtctgctgccttcgatttctcccgatgaactg
atgcaaggatttgatgatgagtgatacccgtatcgagcg
FpvA Fur regulated promoter region (Fur repressor
site underlined)
(SEQ ID NO: 70)
tccggcgaattttctacacagagctgctgccggacctcaagcgcctgggc
aagaccatcatcgtgataagccacgacgaccgctacttcgacgtcgccga
ccagctcatccacatggcggcaggcaaggtccaacaggagaaccgcgtcg
cagattgcatttaatttttccggttttggccgatgagtgcgtcccaatca
ataacaagaattaatactattaacatctgacactcaagggctttgaaaaa
OmpR-1 Fur regulated promoter region (Fur
repressor site underlined)
(SEQ ID NO: 71)
caggtagcgcaggcgctcttccaggtggcgcaactgagtgtcgtcaaggc
taccggtcacttccttgcgatagcgggcgatgaagggcacggtcgagcct
tcgtccaacaggctcacggccgcctcgacctgctgcgggcgtacgcccag
ttcctcggcgatacggctgttgatgctgtccatgtaaaccacctgacatt
tgtgaatacgggggtcgcctgtgggctttttgcccggcggcgctggatga
aagccgcgcattatacccatcgcaaacggcttgcggtgatggcgcccggc
cagccggaactggcgccgggggaaaaatctgctaacaatgctcacgcaac
gtgcagcaatggctacgccataatgcgcggcgatatcagaggagttattc
Fur repressor binding sites of fumC-1 promoter
(SEQ ID NO: 65)
aaacatgaaggacatgttc
(SEQ ID NO: 66)
aataatgaatatcattatc
Fur repressor binding sites of fpvA promoter
(SEQ ID NO: 67)
aataacaagaattaatact
Fur repressor binding sites of ompR-1 promoter
(SEQ ID NO: 68)
cataatgcgcggcgatatc

Fur regulated promoters and their Fur repressor sites have been described and characterized from many non-Pseudomonas species and are listed and reviewed by Carpenter et al. (2009). Fur binding can vary considerably between different genera. For example, the consensus Fur binding site for E. coli is GATAATGATAATCATTATC (de Lorenzo et al. 1987) while the consensus Fur binding site for B. subtilis is TGATAATTATTATCA (Baichoo and Heimann, 2002).

### REFERENCES

Altschul, S. F., Gish, W., Miller, W., Myers, E. W. & Lipman, D. J. (1990) “Basic local alignment search tool.” J. Mol. Biol. 215:403-410.
Baichoo N, Heimann J D. (2002) Recognition of DNA by Fur: a reinterpretation of the Fur box consensus sequence. J. Bacteriol. 184(21):5826-32.
Baichoo N, Wang T, Ye R, Heimann J D. (2002) Global analysis of the Bacillus subtilis Fur regulon and the iron starvation stimulon. Mol. Microbiol. 45(6):1613-29.
Barton H A, Johnson Z, Cox C D, Vasil A I, Vasil M L. (1996) Ferric uptake regulator mutants of Pseudomonas aeruginosa with distinct alterations in the iron-dependent repression of exotoxin A and siderophores in aerobic and microaerobic environments. Mol. Microbiol. 21(5):1001-17.
Binz, T. M., Wenzel, S. C., Schbell, H., Bechthold, A., Müller, R. (2008) Heterologous expression and genetic engineering of the phenalinolactone biosynthetic gene cluster by using Red/ET recombineering. Chem Bio Chem. 9: 447-454.
Carpenter B M, Whitmire J M, Merrell D S. (2009) This is not your mother's repressor: the complex role of fur in pathogenesis. Infect Immun. 77(7):2590-601.
de Lorenzo V, Wee S, Herrero M, Neilands J B. (1987) Operator sequences of the aerobactin operon of plasmid CoIV-K30 binding the ferric uptake regulation (fur) repressor. J. Bacteriol. 169(6):2624-30.
Garrity, P. A., Ligation-Mediated PCR, in PCR 2 A Practical Appraoch, McPherson, M. J. et. al. (Eds.) pp. 309-322 Oxford University Press, New York (1995).
Gish, W. & States, D. J. (1993) “Identification of protein coding regions by database similarity search.” Nature Genet. 3:266-272.
Gu, J. Q., Nguyen, K. T., Gandhi, C., Rajgarhia, V., Baltz, R. H., Brian, P., Chu, M. (2007) Structural characterization of daptomycin analogues A21978C1-3(d-Asn11) produced by a recombinant Streptomyces roseosporus strain. J. Nat. Prod. 70: 233-240.
Hassett D J, Howell M L, Ochsner U A, Vasil M L, Johnson Z, Dean G E. (1997) An operon containing fumC and sodA encoding fumarase C and manganese superoxide dismutase is controlled by the ferric uptake regulator in Pseudomonas aeruginosa: fur mutants produce elevated alginate levels. J. Bacteriol. 179(5):1452-9.
Heim S, Ferrer M, Heuer H, Regenhardt D, Nimtz M, Timmis K N. (2003) Proteome reference map of Pseudomonas putida strain KT2440 for genome expression profiling: distinct responses of KT2440 and Pseudomonas aeruginosa strain PAO1 to iron deprivation and a new form of superoxide dismutase. Environ Microbiol. 5(12):1257-69.
Higgins D. G., Thompson J. D., Gibson T. J. (1996). Using CLUSTAL for multiple sequence alignments. Methods Enzymol., 266, 383-402.
Finking, R., Marahiel, M A., (2004) Biosynthesis of nonribosomal polypeptides. Annu Rev. Microbiol. 58: 453-488.
Marahiel, M. A., Stachelhaus, T., Mootz, H. D. (1997) Modular peptide synthetases involved in nonribosomal peptide synthesis. Cem. Rev. 97:2651-2673.
Pfeifer, B. A., Admiraal, S. J., Gramajo, H., Cane, D. E., Khosla, C. (2001) Biosynthesis of complex polyketides in a metabolically engineered strain of E. coli. Science. 291: 1790-1792.
Rausch, C., Weber, T., Kohlbacher, O. Wohlleben, W., Huson, D. H. (2005) Specificity prediction of adenylation domains in nonribosomal peptide synthetases (NRPS) using transductive support vector machines (TSVMs). Nuc. Acids Res. 33: 5799-5808.
Rouhiainen, L., Paulin, L., Suomalainen, S., Hyytiainen, H., Buikema, W., Haselkorn, R., Sivonen, K. (2000) Genes encoding synthetases of cyclic depsipeptides, anabaenopeptilides, in Anabaena strain 90. Mol. Microbiol. 37: 156-167.
Sambrook, J., Maniatis, T. (1989) Molecular Cloning: A Laboratory Manual. Cold Spring Harbor Laboratory Press. Cold Spring Harbor N.Y.
Shaying Zhao (Ed.), Marvin Stodolsky, Marvin Stodolsky (Ed.) (2003) Bacterial Artificial Chromosomes (Methods in Molecular Biology Series v255-256): Library Construction, Physical Mapping, and Sequencing, Vol. 1 Springer-Verlag New York, LLC.
Shen, B. (2004) Accessing natural products by combinatorial biosynthesis. Sci STKE. Pe14.
Stachelhaus, T. Mootz, H. D., Marahiel, M. A. (1999) The specificity-conferring code of adenylation domains in nonribosomal peptide synthetases. Chem. Biol. 6: 493-505.
Staunton, J., Weissman, K. J. (2001) Polyketide biosynthesis: a millennium review. Nat. Prod. Rep. 18:380-416.
Wenzel, S. C., Müller, R. (2005) Recent developments towards the heterologous expression of complex bacterial natural product biosynthetic pathways. Curr. Op. Biotechnol. 16: 594-606.
Von Dohren, H., Keller, U., Vater, J., and Zocher, R. (1997) Multifunctional peptide synthetases. Chem. Rev. 97:2675-2705.
Zhang, Y., Muyrers, J. Testa, G., Stewart, F. (1998) A new logic for DNA engineering using recombination in Escherichia coli. Nat. Genet. 20: 123-128.
Zhuang, H., Yong, W., Pfeifer, B. A. (2008) Bacterial hosts for natural product production. Molecular Pharmaceuticals. 5: 212-225.

1. A polynucleotide comprising one or more functional fragments of a biosynthetic gene cluster encoding NRPS2, a non ribosomal peptide synthase involved in the production of a compound of:
(a) formula (I) 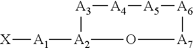
wherein the ester bond is found between the carboxy group of A7 and the hydroxy group of A2, and, optionally, the nitrogen atom of the amid bond between A5 and A6 is substituted with a methyl wherein X and A₁ are each independently optional, and wherein
X is any chemical residue, particularly H or an acyl residue, particularly CH₃CH₂CH(CH₃)CO, (CH₃)₂CHCH₂CO or (CH₃)₂CHCO
A₁ is a standard amino acid which is not aspartic acid, particularly glutamine;
A₂ is threonine or serine particularly threonine;
A₃ is a non-basic standard amino acid or a non-basic derivative thereof, particularly leucine;
A₄ is Ahp, dehydro-AHP, proline or a derivative thereof, particularly Ahp or a derivative thereof, particularly the Ahp derivative 3-amino-2 piperidone;
A₅ is isoleucine or valine, particularly isoleucine;
A₆ is tyrosine or a derivative thereof, particularly tyrosine;
A₇ is leucine, isoleucine or valine, particularly isoleucine or valine, particularly isoleucine; or
(b) formula (I′) 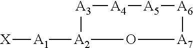
wherein the ester bond is found between the carboxy group of A7 and the hydroxy group of A2, and, optionally, the nitrogen atom of the amid bond between A5 and A6 is substituted with a methyl, wherein
X is CH₃CO, (CH₃)₂CHCO, CH₃S(O)CH₂CO, CH₃CH₂CH(CH₃CO or C₆H₅CO
A₁ is glutamine;
A₂ is threonine;
A₃ is leucine;
A₄ is Ahp, dehydro-AHP, proline or 5-hydroxy-proline;
A₅ is isoleucine or valine, particularly isoleucine;
A₆ is tyrosine;
A₇ is isoleucine or valine, particularly isoleucine.
said polynucleotide comprising:
(i) a nucleotide sequence that has at least 80% sequence identity to a sequence selected among the group consisting of SEQ ID NO: 1, 3, 5, 7, 9, 11, 13, 46, 48, 50, 52, 54, 56, 58 and 60 encoding a NRPS2 domain and/or the complement thereof;
(iii) a nucleotide sequence encoding an amino acid sequence that has at least 90% sequence identity to a sequence selected among the group consisting of SEQ ID NO: 2, 4, 6, 8, 10, 12, 14, 47, 49, 51, 53, 55, 57, 59 or 61 representing a NRPS2 domain and/or the complement thereof; or
(v) a nucleotide sequence that has at least 80% sequence identity to a sequence selected among the group consisting of SEQ ID NO: 15, SEQ ID NO:28 and/or the complement thereof;
wherein said nucleotide sequences according to (i) to (iii) encode an expression product which retains the activity of the corresponding NRPS domain(s) represented by the reference sequence(s) of SEQ ID NOs: 2, 4, 6, 8, 10, 12, 14, 47, 49, 51, 53, 55, 59 and/or 61.

2. A polynucleotide according to claim 1, which encodes an expression product which retains the activity of one or more of the following NRPS2 domains:
(i) the thiolation domain of SEQ ID NO:47;
(ii) the condensation domain of SEQ ID NO:49;
(iii) the adenylation domain for Proline of SEQ ID NO:51;
(iv) the thiolation domain of SEQ ID NO:53;
(v) the condensation domain of SEQ ID NO:2
(vi) the adenylation domain for isoleucine of SEQ ID NO:4;
(vii) the thiolation domain of SEQ ID NO:6;
(viii) the condensation domain of SEQ ID NO:8
(ix) the adenylation domain for tyrosine of SEQ ID NO:10;
(x) the N-methylation domain of SEQ ID NO:12;
(xi) the thyolation domain of SEQ ID NO:14;
(xii) the condensation domain of SEQ ID:55;
(xiii) the adenylation domain for isoleucine of SEQ ID NO:57;
(xiv) the thiolation domain of SEQ ID NO:59; and,
(xv) the thioesterase domain of SEQ ID NO61.

3. A polynucleotide comprising one or more functional fragments of a biosynthetic gene cluster encoding NRPS1, a non ribosomal peptide synthase involved in the production of a compound of:
(a) formula (I) 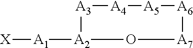
wherein the ester bond is found between the carboxy group of A7 and the hydroxy group of A2, and, optionally, the nitrogen atom of the amid bond between A5 and A6 is substituted with a methyl wherein X and A₁ are each independently optional, and wherein
X is any chemical residue, particularly H or an acyl residue, particularly CH₃CH₂CH(CH₃)CO, (CH₃)₂CHCH₂CO or (CH₃)₂CHCO
A₁ is a standard amino acid which is not aspartic acid, particularly glutamine;
A₂ is threonine or serine, particularly threonine;
A₃ is a non-basic standard amino acid or a non-basic derivative thereof, particularly leucine;
A₄ is Ahp, dehydro-AHP, proline or a derivative thereof, particularly Ahp or a derivative thereof, particularly the Ahp derivative 3-amino-2 piperidone;
A₅ is isoleucine or valine, particularly isoleucine;
A₆ is tyrosine or a derivative thereof, particular tyrosine;
A₇ is leucine, isoleucine or valine, particular isoleucine or valine, particularly isoleucine; or
(b) formula (I′) 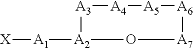
wherein the ester bond is found between the carboxy group of A7 and the hydroxy group of A2, and, optionally, the nitrogen atom of the amid bond between A5 and A6 is substituted with a methyl, wherein
X is CH₃CO, (CH₃)₂CHCO, CH₃S(O)CH₂CO, CH₃CH₂CH(CH₃)CO or C₆H₅CO
A₁ is glutamine;
A₂ is threonine;
A₃ is leucine;
A₄ is Ahp, dehydro-AHP, proline or 5-hydroxy-proline;
A₅ is isoleucine or valine, particular isoleucine;
A₆ is tyrosine;
A₇ is isoleucine or valine, particularly isoleucine.
said polynucleotide comprising:
(i) a nucleotide sequence that has at least 80% sequence identity to a sequence selected among the group consisting of SEQ ID NO: 30, 32, 34, 36, 38, 40, 42 and 44 encoding a NRPS1 domain and/or the complement thereof;
(iii) a nucleotide sequence encoding an amino acid sequence that has at least 90% sequence identity to a sequence selected among the group consisting of SEQ ID NO: 31, 33, 35, 37, 39, 41, 43, 45 representing a NRPS1 domain and/or the complement thereof;
(v) a nucleotide sequence that has at least 80% sequence identity to a sequence selected among the group consisting of SEQ ID NO: 26 and/or the complement thereof; or
wherein said nucleotide sequences according to (i) to (iii) still encode an expression product which retains the activity of the corresponding NRPS domain(s) represented by the reference sequences of SEQ ID NOs: SEQ ID NO: 31, 33, 35, 37, 39, 41, 43, 45.

4. A polynucleotide according to claim 3, which encodes an expression product which retains the activity of the one or more of following NRPS1 domains:
(i) the loading domain of SEQ ID NO:31;
(ii) the adenylation domain for glutamine of SEQ ID NO:33;
(iii) the thiolation domain of SEQ ID NO:35;
(iv) the condensation domain of SEQ ID NO:37;
(v) the adenylation domain for threonine of SEQ ID NO:39;
(vi) the thiolation domain of SEQ ID NO:41;
(vii) the condensation domain of SEQ ID NO:43; and,
(viii) the adenylation domain for leucine of SEQ ID NO:45.

5. A polynucleotide encoding a NRPS2 for producing a compound of formula (I) or (I′) comprising a nucleotide sequence encoding an amino acid sequence as depicted in SEQ ID NO:29.

6. A polynucleotide encoding a NRPS1 for producing a compound of formula (I) or (I′) comprising a nucleotide sequence encoding an amino acid sequence as depicted in SEQ ID NO: 27.

7. A polypeptide encoded by one or more polynucleotide(s) of claim 1.

8. A polypeptide involved in the production of a compound of formula (I) or (I′) comprising an amino acid sequence selected from the group consisting of:
(i) SEQ ID NO:27 representing a first NRPS1, SEQ ID NO:29 representing a second NRPS2, SEQ ID NO:63 representing a cytochrome P450; and,
(ii) a functional variant of an amino acid sequence listed in (i), having at least 90% and retaining substantially the same catalytic function.

9. A polynucleotide comprising a nucleotide sequence encoding one or more polypeptides involved in the production of a compound of formula (I) or (I′) and comprising an amino acid selected from the group consisting of:
(i) SEQ ID NO:27 representing a first NRPS1, SEQ ID NO:29 representing a second NRPS2, SEQ ID NO:63 representing a cytochrome P450; and
(ii) a functional variant of an amino acid sequence listed in (i), having at least 90%, sequence identity and retaining substantially the same catalytic function.

10. A polynucleotide comprising a nucleotide sequence encoding polypeptides for the production of a compound of formula (I) and (I′) comprising:
(i) a nucleotide sequence encoding SEQ ID NO:27 or a functional variant thereof; and
(ii) a nucleotide sequence encoding SEQ ID NO:29 or a functional variant thereof; and
(ii) a nucleotide sequence encoding SEQ ID NO:63 or a functional variant thereof representing cytochrome p450.

11. (canceled)

12. The polynucleotide of claim 10, isolated from the Chondromyces crocatus strain NPH-MB180 having accession number DSM 19329.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A polypeptide encoded by one or more polynucleotide(s) of claims 3.

28. A polypeptide encoded by the polynucleotide of claims 5.

29. A polypeptide encoded by the polynucleotide of claims 6.

30. An expression vector comprising a polynucleotide sequence encoding one or more polypeptides for producing a compound of formula (I) or (I′) and which polypeptides comprise an amino acid sequence of:
(i) SEQ ID NO:27 representing a first NRPS1, SEQ ID NO:29 representing a second NRPS2, and SEQ ID NO:63 representing a cytochrome P450; or
(ii) a functional variant of an amino acid sequence listed in (i), having at least 90% sequence identity and retaining substantially the same catalytic function;
wherein the open reading frames are operatively linked with transcriptional and translational sequences.

31. An expression vector comprising a polynucleotide sequence encoding one or more polypeptides for producing a compound of formula (I) or (I′) comprising:
(i) a nucleotide sequence encoding SEQ ID NO:27 or a functional variant thereof; and
(ii) a nucleotide sequence encoding SEQ ID NO:29 or a functional variant thereof; and
(iii) a nucleotide sequence encoding SEQ ID NO:63 or a functional variant thereof representing a cytochrome P450;
wherein the open reading frames are operatively linked with transcriptional and translational sequences.

32. The expression vector of claim 30 further comprising a nucleotide sequence encoding one or more open reading frames selected among the group consisting of SEQ ID NO:16, SEQ ID NO:18, SEQ ID NO:20, SEQ ID NO:22 and SEQ ID NO:24, or their functional variant.

33. A host cell comprising one or more recombinant polynucleotides according to claim 1, wherein said nucleotide sequence is not naturally found in the genome of said host cell

34. A host cell comprising one or more recombinant polynucleotides according to claim 3, wherein said nucleotide sequence is not naturally found in the genome of said host cell.

35. A host cell comprising one or more recombinant polynucleotides according to claim 9, wherein said nucleotide sequence is not naturally found in the genome of said host cell.

36. A host cell comprising an expression vector according to claim 30 wherein said nucleotide sequence is not naturally found in the genome of said host cell.

37. A host cell comprising an expression vector according to claim 31, wherein said nucleotide sequence is not naturally found in the genome of said host cell.

38. A host cell comprising an expression vector according to claim 32, wherein said nucleotide sequence is not naturally found in the genome of said host cell.

39. A host cell according to claim 36 for producing a compound of formula (I) or (I′) or of formula (II) to (VII), (XI) to (XIV) and (XVII) and (XVIII).

40. A host cell according to claim 38 for producing a compound of formula (I) or (I′) or of formula (II) to (VII), (XI) to (XIV) and (XVII) and (XVIII).

41. The host cell according to claim 36, wherein the recombinant polynucleotides have been modified for optimized gene expression.

42. The host cell according to claim 36, wherein the codon usage of the polynucleotide has been adjusted to the codon usage of abundant proteins of the host cell.

43. The host cell according to claim 36, wherein said host cell is selected from species of the order Myxococcales or the genera Pseudomonas or Streptomyces.

44. The host cell according to claim 36, wherein said host cell is selected among Pseudomonas putida species.

45. A mutant microorganism, wherein said mutant microorganism no longer expresses a gene required for the production of a compound of formula (I) or (I′).

46. The mutant microorganism of claim 45, wherein said mutant microorganism no longer expresses a gene encoding a cytochrome P450 as depicted in SEQ ID NO: 63.

47. A method of preparing a compound of formula (I) or (I′) or of formula (II) to (VII), (XI) to (XIV) or (XVII) to (XVIII), comprising culturing a host cell according to claim 36 under conditions such that said compound is produced.
